(19) United States
(12) Patent Application Publication
(10) Pub. No.: US 2023/0106519 A1
(43) Pub. Date: Apr. 6, 2023

(54) GOLD(III)-BIGUANIDE PRODRUGS, METHODS OF PREPARATION AND USES THEREOF

(71) Applicants: National University of Singapore, Singapore (SG); Northwestern University, Evanston, IL (US)

(72) Inventors: Wee Han Ang, Singapore (SG); Maria Babak, Singapore (SG); Irina Vadimovna Balyasnikova, Chicago, IL (US)

(73) Assignees: National University of Singapore, Singapore (SG); Northwestern University, Evanston, IL (US)

(21) Appl. No.: 17/907,238

(22) PCT Filed: Mar. 23, 2021
(86) PCT No.: PCT/SG2021/050160

§ 371 Date: Sep. 23, 2022

(30) Foreign Application Priority Data
Mar. 23, 2020 (SG) 10202002670Q

(51) Int. Cl. C07F 1/12; A61P 35/00

(52) U.S. Cl. CPC C07F 1/12; A61P 35/00

(57) ABSTRACT

The present invention relates to a compound of following formula (I), which is a novel prodrug of biguanide derivatives: (I). The present invention also relates to the preparation and methods of using the compound. In particular, the invention relates to prodrugs wherein metformin and phenformin are attached to Au^{III} cyclometalated backbone to form novel Au^{III} prodrugs of metformin or phenformin with improved anticancer effects.

### FIELD OF THE INVENTION

The present invention belongs to the field of pharmaceutical chemistry and relates to novel prodrugs of biguanide derivatives, their preparation and methods of using the prodrugs. In particular, the invention relates to prodrugs wherein metformin and phenformin are attached to Au^{III} cyclometalated backbone to form novel Au^{III} prodrugs of metformin or phenformin with improved anticancer effects.

### BACKGROUND OF THE INVENTION

Metformin and its less polar analogue phenformin belong to a family of biguanides that are widely prescribed as over-the-counter antidiabetic medications. Metformin in particular is a first-line treatment for Type II diabetes and is listed as one of World Health Organization (WHO) essential medicines. The anticancer role of metformin was recognized after retrospective epidemiological analyses, which revealed that it significantly reduced cancer risks in diabetic patients, particularly breast cancer.

The anticancer activity of metformin and phenformin has been linked to their ability to alter cancer cell metabolism. Cancer cells progressively modify normal cellular functions in order to promote cell growth, support rapid proliferation and disable cell death mechanisms and immune surveillance. Acceleration of normal cell division requires metabolic adjustments to provide cancer cells with the additional energy; hence, they switch their main energy production from the oxidative phosphorylation (OXPHOS) to the less efficient aerobic glycolysis. However, the loss of ATP is counterbalanced by a higher glycolytic rate and increased glucose uptake. Since constant energy supply is paramount to cancer cells' survival, the interference with their energy production results in a metabolic catastrophe which inevitably leads to cancer cell death. Metformin and phenformin target energy production in cancer cells by inhibiting Complex I of the mitochondrial respiratory chain, activating 5′-adenosine monophosphate-activated protein kinase (AMPK) and lowering body insulin levels by altering insulin/insulin-like growth factor-I (I/IGF) pathway, thereby inactivating P13K/Akt/mTOR pathways and inhibiting tumor cell proliferation. In addition, metformin inhibits Complex I of the mitochondrial respiratory chain and targets OXPHOS. Further, metformin and phenformin have been repeatedly shown to enhance antiproliferative effects of other drugs, including cisplatin, 2-deoxyglucose, doxorubicin, and tamoxifen in a synergistic manner both in vitro and in vivo.

Despite its well-characterized antiproliferative activity and low cost, the use of metformim as an anticancer agent has some serious drawbacks. According to Biopharmaceutics Classification System (BCS) and Biopharmaceutics Drug Disposition Classification System (BDDCS) metformin is classified as a Class 3 compound, indicating its high aqueous solubility and low permeability across cellular membranes. Under physiological pH metformin is cationic and acts as a hydrophilic base; therefore, its passive diffusion across cell membranes is very limited. Due to its poor cellular uptake, its anticancer effects in vitro are observed only at high millimolar concentrations. Similarly, in cancer patients, metformin and phenformin demonstrated anticancer effects only when taken repeatedly in high doses because of its short biological half-life and low absorption. Such high doses can cause adverse side-effects, including lactic acidosis, and are not compatible with chemotherapy regimens.

Phenformin is less polar than metformin and exhibits higher affinity for mitochondrial membranes, which is reflected by its anticancer activity in a micromolar concentration range. Indeed, repeated use of phenformin at high doses is associated with fatal lactic acidosis, leading to its withdrawal from the market. Therefore, a need exists for compounds that are effective against cancer cells and tumors with increased permeability and efficacy and reduced toxic effects.

Various strategies have been employed to overcome the difficulties with administering metformin and phenformin at high doses. Drug encapsulation into delivery systems can significantly improve their delivery into cancer cells and reduce side-effects. Conjugation of metformin with mitochondria-targeting triphenylphosphonium cation resulted in a marked increase of in vitro cytotoxicity up to low micromolar range. Various formulation strategies have been exploited, including extended-release formulations to improve the malabsorption of metformin. Additionally, several organic prodrugs of metformin have been prepared, which improved the oral availability of the drug.

Prodrugs are pharmacologically inactive or impaired derivatives of drug molecules aimed to improve the unfavourable physicochemical, pharmaceutical or biopharmaceutical properties of a parent drug. Amino acid derivatives of metformin which are more likely amides are known. Moreover, metformin linked with a carrier, in this case an amino acid arginine, via a linker group is also known. However, all the previously described prodrugs were developed simply to improve the bioavailability of metformin, metformin prodrugs where other parts of the molecule synergistically act with metformin parent drug, thereby increasing its antitumor effect, have never been described. In addition, the chemical conjugation of metformin and phenformin with another active pharmacophore has never been explored so far.

Therefore, there is a need to provide alternative compounds that can act as successfully facilitate the treatment of cancer with metformin derivatives.

### SUMMARY

In an aspect, there is provided a compound of formula (I):

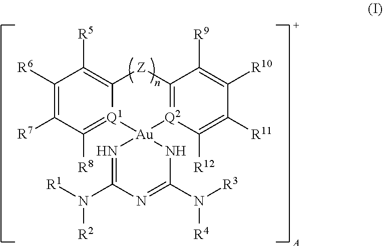

wherein,
n may be 0 or 1;
Z may be C, N, O, S, Si, C═O or C═S,
Q¹ and Q² for each occurrence may be independently selected from C, P or N, wherein at least one of Q¹ and Q² is N;
R¹, R², R³ and R⁴ for each occurrence may be independently selected from the group consisting of hydrogen, optionally substituted alkyl, optionally substituted alkenyl and optionally substituted alkynyl, provided that at least one of R¹, R², R³ or R⁴ is an optionally substituted alkyl;
R⁵, R⁶, R⁷, R⁸, R⁹, R¹⁰, R¹¹ and R¹² for each occurrence may be independently selected from the group consisting of hydrogen, —X, —OR′, —SR′, —P(R′)₂, —C(═O)OR′, C(═O)R′, —COX, —CX₃, —NO₂, —SO₃H, —SO₂R′, —N═O, optionally substituted alkyl, optionally substituted amino, optionally substituted alkyloxy, optionally substituted cycloalkyl, optionally substituted heterocycloalkyl, optionally substituted aryl, and optionally substituted heteroaryl, wherein R′ is independently hydrogen or an optionally substituted alkyl and X is halogen; and
A may be tosylate (OTs⁻), mesylate (OMs⁻), BF₄⁻, NO₃⁻, Cl⁻, Br⁻, triflate (OTf⁻), ClO₄⁻, bistriflimide (TFSI⁻), BPH₄⁻, BPh₄^{F-}, tetrakis(1-imidazolyl)borate (Blm₄⁻), tetrakis(2-thienyl)borate (BTH₄⁻), HPO₄²⁻ or PF₆.

Advantageously, the compound as defined above may be a prodrug comprising a biguanide derivative such as metformin or phenformin coordinated to an Au^{III} complex. Advantageously, biguanide derivatives such as metformin may be energy-disrupting. Biguanide derivatives may therefore have anticancer properties and may enhance antiproliferative effects of other drugs. In the hypoxic conditions of cancer cells, Au^{III} complexes may be activated either by reduction or ligand substitution mechanisms and may exhibit excellent anticancer activity in vitro and in vivo. Further, Au^{III} complexes may inhibit the thioredoxin reductase (TrxR) enzyme, selected zinc-finger domains and protein tyrosine phosphatases (PTP), and therefore may also have anticancer properties of their own. Consequently, both Au^{III} and metformin may induce cellular metabolic perturbations. Further advantageously, the compound as defined above may assist in the delivery of the biguanide derivative to a target site such as a cancer cell and release the biguanide derivative specifically in the cancer cell. Advantageously, the biguanide derivative may be deposited in the cancer cell via ligand exchange. This may result in both Au and the biguanide derivative being deposited within the target cancer cell, which may ultimately lead to the death of the target cancer cell by the Au and biguanide derivatives acting synergistically. Advantageously, as both metformin and Au^{III} fragments may affect mitochondria by interfering with different pathways, chemical attachment of metformin and phenformin to an Au^{III} center may result in synergistic action of both fragments in cancer cells.

Further advantageously, the compound as defined above is stable in aqueous media, making them suitable for in vivo use. In addition, in comparison with metformin on its own, a significant advantage of the compound as defined above is their improved lipophilicity, which ensures efficient intracellular accumulation.

Advantageously, the compound as defined above may have excellent cytotoxicity profiles in the micromolar to nanomolar concentration range in a variety of cell lines, including highly resistant triple-negative breast cancer MDA-MB-231 cells. The compound as defined above may be more active than cisplatin. Further advantageously, the compound as defined above may have significantly higher cytotoxicity than the respective precursors of the compounds (that is, the Au^{III} complex without the biguanide derivative coordinated, or the biguanide derivative alone), suggesting that the coordination of the biguanide derivative onto the Au^{III} complex may synergistically increase the cytotoxicity. Further, this was corroborated by significantly higher cellular uptake observed by the compound as defined above.

Further advantageously, the coordination of the biguanide derivative to the Au^{III} complex may reduce the toxicity of Au complexes. The compound as defined above may demonstrate only marginal toxicity to the liver and heart or may not be toxic at all.

More advantageously, the compound as defined above may significantly inhibit mitochondrial respiration, which may be related to the synergistic action of the biguanide derivatives and the Au^{III} complex. The compound as defined above may efficiently inhibit TrxR enzyme, which may lead to the disruption of mitochondrial functions.

Further advantageously, it was shown that metabolic changes caused by the compound as defined above may initiate the cancer cell to attempt to protect themselves by metabolic reprogramming, unfolded protein response (UPR) and mitophagy, which may be successfully prevented by the compound as defined above by shutting down of the mitochondrial respiration and impairment of autophagic flux. That is, the compound as defined above may induce endoplasmic reticulum (ER) stress and autophagy at nanomolar concentrations. This may lead to the inhibition of protein degradation and apoptotic cell death.

Furthermore, the compound as defined above advantageously exhibited a high degree of selectivity to cancer cells over healthy heart and liver cells.

In another aspect, there is provided a method for preparing a compound as defined above, the method comprising the step of contacting:
a compound having the following formula (IV):

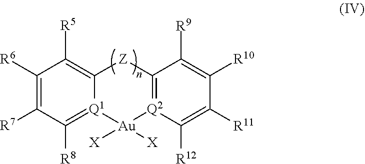

with a compound having the following formula (V):

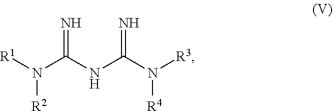

wherein
n may be 0 or 1;
Z may be C, N, O, S, Si, C═O or C═S,
Q¹ and Q² for each occurrence may be independently selected from C, P or N, wherein at least one of Q¹ or Q² is N;
R¹, R², R³ and R⁴ for each occurrence may be independently selected from the group consisting of hydrogen, optionally substituted alkyl, optionally substituted alkenyl, and optionally substituted alkynyl, provided that at least one of R¹, R², R³ or R⁴ is an optionally substituted alkyl;
R⁵, R⁶, R⁷, R⁸, R⁹, R¹⁰, R¹¹ and R¹² for each occurrence may be independently selected from the group consisting of hydrogen, —X, —OR′, —SR′, —P(R′)₂, —C(═O)OR′, C(═O)R′, —COX, —CX₃, —NO₂, —SO₃H, —SO₂R′, —N═O, optionally substituted alkyl, optionally substituted amino, optionally substituted alkyloxy, optionally substituted cycloalkyl, optionally substituted heterocycloalkyl, optionally substituted aryl, and optionally substituted heteroaryl, wherein R′ is independently hydrogen or an optionally substituted alkyl and X is halogen.

Advantageously, the method as defined above may provide a facile and efficient synthesis for the preparation of the compound as defined above.

In another aspect, there is provided a pharmaceutical composition comprising a compound as defined above and a pharmaceutically acceptable excipient.

In another aspect, there is provided a compound as defined above or pharmaceutical composition as defined above for use as a medicament.

In another aspect, there is provided the use of a compound as defined above or pharmaceutical composition as defined above in the manufacture of a medicament for the treatment of cancer.

Advantageously, the compound as defined above may be useful as a medicament for the treatment of cancer. Specifically, the combination of the Au^{III} complex and the biguanide derivative may result in each component mitigating the short-comings of the other, and facilitating a synergistic effect of high efficacy and low toxicity in the treatment of cancer.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The invention will be better understood with reference to the detailed description when considered in conjunction with the non-limiting examples and the accompanying drawings, in which:

FIG. 1 shows the chemical structures of metformin, phenformin and Au^{III} complexes disclosed.

FIG. 2 shows a scheme outlining the synthesis of cyclometalated Au^{III} complexes with metformin.

FIG. 3 shows the ¹H NMR spectrum of metformin hydrochloride in DMSO-d⁶.

FIG. 4 shows the ¹H NMR spectrum of phenformin hydrochloride in DMSO-d⁶.

FIG. 5 shows the ¹H NMR spectrum of compound 2 in DMSO-d⁶.

FIG. 6 shows the ¹H NMR spectrum of compound 3 in DMSO-d⁶.

FIG. 7 shows the ¹H NMR spectrum of 1met* in D₂O (the signals from the minor isomer are depicted by circles).

FIG. 8 shows the ¹H NMR spectrum of 1met in DMSO-d⁶.

FIG. 9 shows the ¹H NMR spectrum of 1phen in DMSO-d⁶ (the signals from the minor isomer are depicted by circles).

FIG. 10 shows the ¹H NMR spectrum of 2met in DMSO-d⁶ (the signals from the minor isomer are depicted by circles).

FIG. 11 shows the ¹H NMR spectrum of 3met in DMSO-d⁶ (the signals from the minor isomer are depicted by circles)

FIG. 12 shows the ¹H NMR study on the stability of 1met in DMSO-d⁶ for 9 days.

FIG. 13 shows the ¹H NMR study on the stability of 1phen in DMSO-d⁶ for 10 days.

FIG. 14 shows the ¹H NMR study on the stability of 2met in DMSO-d⁶ for 6 days.

FIG. 15 shows the RP-HPLC analysis of compound 1.

FIG. 16 shows the RP-HPLC analysis of compound 2.

FIG. 17 shows the RP-HPLC analysis of compound 3.

FIG. 18 shows the RP-HPLC analysis of 1met.

FIG. 19 shows the RP-HPLC analysis of 1phen.

FIG. 20 shows the RP-HPLC analysis of 2met.

FIG. 21 shows the RP-HPLC analysis of 3met.

FIG. 22 shows the high resolution ESI-MS spectrum of 1met.

FIG. 23 shows the high resolution ESI-MS spectrum of 2met.

FIG. 24 shows the high resolution ESI-MS spectrum of 3met

FIG. 25 shows the high resolution ESI-MS spectrum of 1phen.

FIG. 26 refers to a graph showing the dose-response curves of MDA-MB-231 cells treated with Au^{III}-metformin and phenformin complexes for 72 hours.

FIG. 27 refers to a graph showing the dose-response curves of MDA-MB-231 cells treated with the lead complex 3met or complex 3 and metformin for 72 hours.

FIG. 28 refers to concentration-effect curves for 1-3met, 1phen and 1met* in (A) A2780, (B) A2780cis and (C) MDA-MB-231 cells lines upon 72 hour exposure.

FIG. 29 refers to excerpts of the high-resolution mass spectra of (A) 1met-PF₆, (B) 2met and (C) 3met incubated. (ii) indicates the complexes incubated in ammonium carbonate for 24 hours and (i) indicates the simulated isotopic patterns provided for comparison. The detection of the molecular ion [M]⁺ peaks indicated that compounds 1-3met were stable for 24 hours.

FIG. 30 refers to (i) simulated isotopic pattern provided for comparison, and (ii) an excerpt of the high-resolution mass spectrum of 1phen incubated in ammonium carbonate for 1 hour. The molecular ion [M]⁺ peak for 1phen was not detected after 1 hour incubation, indicating lower stability compared to the Au^{III}-metformin analogues 1-3met.

FIG. 31 refers to (i) simulated isotopic pattern provided for comparison, and (ii) an excerpt of the low molecular weight region of the high-resolution mass spectrum of 1phen incubated in ammonium carbonate for 1 hour. No peak corresponding to free phenformin was detected.

FIG. 32 refers to an excerpt of the high-resolution mass spectrum of 1phen incubated in ammonium carbonate with 1 eq. of glutathione for 1 hour. (i) indicates the simulated isotopic pattern of [phenformin+H⁺], (ii) indicates the simulated isotopic pattern of [1phen]+ and (iii) indicates the experimental spectrum. Only the peak at m/z 206.1622, which corresponds to free phenformin, was observed.

FIG. 33 refers to an excerpt of the high-resolution mass spectrum of 3met incubated in ammonium carbonate with and without 1 eq. of glutathione (GSH) for 0, 1, 3 and 24 hours. Without GSH (left side) only background peak at m/z 130.1603 has been detected and no intensity changes has been observed over time. In the presence of GSH (right side) a new peak at m/z 130.1078 has been observed, corresponding to uncoordinated metformin, and its intensity increased more than 5-fold.

FIG. 34 refers to an excerpt of the high-resolution mass spectrum of 3met incubated in ammonium carbonate with and without 1 eq. of GSH for 0, 1, 3 and 24 hours. Without GSH (left side) the peak at m/z 560.1358 corresponding to M⁺ did not change intensity over time. In presence of GSH the intensity of M⁺ peak decreased more than 27-fold.

FIG. 35 refers to quantification of the peak area corresponding to (A) metformin signal at m/z 130.1078 (corresponding to FIG. 33 (right side)) and (B) M⁺ signal at m/z 506.1358 (corresponding to FIG. 34 (right side)).

FIG. 36 refers to the UV-vis spectra of 3met in water at pH7 upon sequential addition (6 times) of 0.4 eq. of glutathione (GSH) in comparison with spectra of GSH and metformin. GSH corresponds to the bottom left line without any visible peaks. Metformin (Met) corresponds to the bottom left line with a peak around 230 nm, 3met corresponds to the solid dark black line with a peak at around 280 nm. The dashed lines correspond to the changes of 3met spectrum upon addition of GSH.

FIG. 37 refers to the UV-vis spectra of 2met in water at pH7 upon sequential addition (6 times) of 0.4 eq. of GSH.

FIG. 38 refers to an excerpt of the high-resolution mass spectrum of 2met incubated in ammonium carbonate with and without 1 eq. of GSH for 0, 1, 3 and 24 hours. The intensity of M+ peak did not significantly change over time.

FIG. 39 refers to UV-vis spectra of (i) 1met with 6 sequential additions of 0.4 eq. of GSH measured at 37° C., (ii) GSH measured at 25° C., (iii) GSH measured at 37° C., (iv) 1met without GSH measured at 25° C. and (v) 1met without GSH measured at 37° C., in aqueous pH7 buffer solution.

FIG. 40 refers to cyclic voltammogram spectra of Au^{III}-metformin and phenformin complexes and uncoordinated metformin in DMSO/nBu₄NPF₆ at potential scan rate of 100 mV s⁻

FIG. 41 refers to UV-vis spectra in 2D projection upon forward scan of in situ UV-vis spectroelectrochemistry of (A) 1met in DMSO/nBu₄NPF₆, and (B) 2met in DMSO/nBu₄NPF₆. The graphs demonstrate the changes in UV-vis spectrum of 1met and 2met upon forward cyclic voltammetry scan. The changes in the spectra occurred in the direction of the bold arrows.

FIG. 42 refers to UV-vis-spectra (scan rate 10 mV s⁻¹) upon cyclic voltammetry forward scan for (A) 1met and (B) 3met in DMSO/nBu₄NPF₆ wherein the following regions are annotated: (i) initial spectrum before the CV scan, (ii) region of change upon scanning and (iii) final spectrum at the end of the scan. The spectra demonstrate the release of metformin.

FIG. 43 refers to a graph showing the dose-response curves of Au^{III} complexes for the inhibition of rat liver TrxR determined by DNTB reduction assay.

FIG. 44 refers to a graph showing the concentration-dependent intracellular accumulation of Au^{III} complexes. MDA-MB-231 cells were exposed to increasing drug concentrations for 24 hours, and the cellular Au content was measured by ICP-MS. Statistical analysis was performed by one-way ANOVA test with Bonferroni post hoc analysis using Graph Pad Prism software (Graph Pad Software Inc., Calif.) with p<0.05 considered as significant (* p<0.05, ** p<0.01, *** p<0.001, ns—not significant).

FIG. 45 refers to a graph showing mitochondrial respiration characterized by oxygen consumption rate (OCR) normalized by protein content in MDA-MB-231 cells treated with indicated concentrations of 3met for 24 hours.

FIG. 46 refers to a graph showing glycolysis characterized by extracellular acidification rate (ECAR) normalized by protein content in MDA-MB-231 cells treated with indicated concentrations of 3met for 24 hours.

FIG. 47 refers to a graph showing mitochondrial characteristics (non-mitochondrial respiration, basal respiration, maximal respiration, proton leak, ATP-linked respiration and spare respiratory capacity) corresponding to the respiration profile in FIG. 45.

FIG. 48 refers to a graph showing glycolysis characteristics corresponding to the glycolysis profile in FIG. 46.

FIG. 49 refers to images showing the Western blot analysis of various proteins in MDA-MB-231 cells treated with indicated concentrations of 1met and 3met for 24 hours. Actin is used as the loading control.

FIG. 50 refers to images showing the Western blot analysis of various proteins in MDA-MB-231 cells treated with indicated concentrations of 1met and 3met for 2, 6 and 24 hours.

FIG. 51 refers to a graph showing cytotoxicity of 3met upon 24 hour co-incubation with 3-MA (2 mM, inhibitor of autophagosome formation) and MDIVI-1 (10 μM, inhibitor of mitochondrial fission).

FIG. 52 refers to images showing the Western blot analysis of AMPK and m-TOR protein phosphorylation in MDA-MB-231 cells treated with (A) increasing concentrations of 1met for 24 hours and (B) 3met for 2, 6 and 24 hours at indicated concentrations.

FIG. 53 refers to a graph showing residual kinase activity (%) after 24 hour co-incubation with 10 μM of 3met.

FIG. 54 refers to an image showing the Western blot analysis of various proteins in MDA-MB-231 cells treated with increasing concentrations of metformin for 24 hours.

FIG. 55 refers to an image showing the Western blot analysis of MDA-MB-231 cells treated with increasing concentrations of 3met for 24 hours in combination with chloroquine (CQ), which was added 1 hour before cell harvesting to prevent autolysosome fusion. Actin was used as the loading control.

FIG. 56 refers to a graph showing the cytotoxicity of 3met upon 24 hours co-incubation with various pharmacological inhibitors.

FIG. 57 refers to a graph showing Annexin V/PI analysis of apoptotic cell fraction (%) in MDA-MB-231 cells treated with compounds (at 1.5×EC₅₀) for 24 hours.

FIG. 58 refers to an image showing the putative mechanism of action of 3met. Green and red arrows indicate upregulation (or activation) and down-regulation (or inhibition) events, respectively, as experimentally determined.

FIG. 59 refers to a graph showing body weight changes of athymic nude mice treated with 3met at 0-20 mg/kg for the determination of the maximum tolerated dose. Only mice treated with 20 mg/kg demonstrated 10% weight loss on day 6.

FIG. 60 refers to a graph showing the growth of MDA-MB-231 tumors from week 1 (before treatment) presented as a fold change in tumor volume. Starting from week 2, tumors became palpable and their volume was measured by calliper weekly. Mice (n=7) were treated with 3met at 15 mg/kg or respective vehicle (DMSO in sterile saline) via i.p. route every other day on weeks 3, 4 and 5. Statistical analysis was performed by one-way ANOVA test with Bonferroni correction using GraphPad Prism 9 software (GraphPad Software Inc., Calif.) with p<0.05 considered as significant (* p<0.05, ** p<0.01, *** p<0.001, ns—not significant).

FIG. 61 refers to a graph showing Au accumulation in mouse organs obtained from 3met-treated mice at the endpoint and quantified by ICP-MS. Statistical analysis was performed by one-way ANOVA test with Dunnett post hoc analysis (vs. tumor) using GraphPad Prism 9 software (GraphPad Software Inc., Calif.) with p<0.05 considered as significant (* p<0.05, ** p<0.01, *** p<0.001, ns—not significant).

FIG. 62 refers to a graph showing weekly body weight changes of athymic nude mice with engrafted MDA-MB-231 breast tumors treated with 3met at 15 mg/kg or corresponding vehicle (DMSO) every other day at weeks 2, 4, and 5 (indicated by arrows). This figure shows that the body weights of the drug treated mice did not change in comparison with the untreated group, indicating that 3met does not cause the loss of weight in treated mice.

FIG. 63 refers to images of hematoxylin & eosin (H&E)-stained liver (A, B, C) and kidney (D, E) tissues of 3met-treated mice (endpoint, 5 weeks). The images showed vacualization, fatty changes, periportal lymphoid infiltration and expansion of Disse space in A and B, and focal necrosis in C. Kidney histopathology showed distorted glomeruli, dilated tubules, edema exudate, light necrosis in D and E. Scale bar in A, D and E represent 100 μm, scale bar in B represents 80 μm and scale bar in C represents 20 μm.

FIG. 64 refers to representative images of H&E-stained tumors in vehicle-(A-D) and 3met-treated (E-G) groups (detailed analysis of the images can be found in Table 6). Scale bars represent 800 μm.

FIG. 65 refers to images of representative H&E-stained tumor tissue of (A) a vehicle-treated mouse, demonstrating grade 3 breast carcinoma with high rate of mitosis, and (B) a 3met-treated mouse, demonstrating wide area of necrosis and strong lymphohistiocytic tumor infiltration. Scale bar for A represents 50 μm and scale bar for B represents 200 μm.

FIG. 66 refers to an image of a representative fragment of H&E-stained tumor tissue of a 3met-treated mouse in FIG. 65B demonstrating lymphocyte infiltration (depicted with white arrows). Scale bar represents 50 μm.

FIG. 67 refers to an image of the tumor tissues after modification with an algorithm for quantification of tumor tissues using QuPath software (random trees pixel classifier, red color (6802) is tumor, black (6804) is necrosis and green (6806) is stroma). The figure demonstrates a general view of the section of the tumor, which was segmented by QuPath software algorithm for viable tumor surrounded by central necrosis and stroma. Scale bar represents 1 mm.

FIG. 68 refers to a graph showing the percentage of necrosis in tumors of vehicle-treated and 3met treated mice. Statistical analysis was performed by one-way ANOVA test with unpaired T test using GraphPad Prism 9 software (GraphPad Software Inc., Calif.) with p<0.05 considered as significant (* p<0.05, ** p<0.01, *** p<0.001, ns—not significant).

### DEFINITIONS

In this specification a number of terms are used which are well known to a skilled addressee. Nevertheless for the purposes of clarity a number of terms will be defined. The following words and terms used herein shall have the meaning indicated:

In the definitions of a number of substituents below it is stated that “the group may be a terminal group or a bridging group”. This is intended to signify that the use of the term is intended to encompass the situation where the group is a linker between two other portions of the molecule as well as where it is a terminal moiety. Using the term alkyl as an example, some publications would use the term “alkylene” for a bridging group and hence in these other publications there is a distinction between the terms “alkyl” (terminal group) and “alkylene” (bridging group). In the present application no such distinction is made and most groups may be either a bridging group or a terminal group.

The term “optionally substituted” as used herein means the group to which this term refers may be unsubstituted, or may be substituted with one or more groups independently selected from acyl, alkyl, alkenyl, alkynyl, thioalkyl, cycloalkyl, cycloalkylalkyl, cycloalkenyl, cycloalkylalkenyl, heterocycloalkyl, cycloalkylheteroalkyl, cycloalkyloxy, cycloalkenyloxy, cycloamino, halo, carboxyl, haloalkyl, haloalkynyl, alkynyloxy, heteroalkyl, heteroalkenyl heteroalkynyl, heteroalkyloxy, hydroxyl, hydroxyalkyl, alkoxy, thioalkoxy, alkenyloxy, haloalkoxy, haloalkenyl, haloalkynyl, haloalkenyloxy, nitro, amino, nitroalkyl, nitroalkenyl, nitroalkynyl, nitroheterocyclyl, alkylamino, dialkylamino, alkenylamine, aminoalkyl, alkynylamino, acyl, alkyloxy, alkyloxyalkyl, alkyloxyaryl, alkyloxycarbonyl, alkyloxycycloalkyl, alkyloxyheteroaryl, alkyloxyheterocycloalkyl, alkenoyl, alkynoyl, acylamino, diacylamino, acyloxy, alkylsulfonyloxy, heterocyclic, heterocycloalkenyl, heterocycloalkyl, heterocycloalkylalkyl, heterocycloalkylalkenyl, heterocycloalkylalkenyl, heterocycloalkylheteroalkyl, heterocycloalkyloxy, heterocycloalkenyloxy, heterocycloxy, heterocycloamino, haloheterocycloalkyl, alkylsulfinyl, alkylsulfonyl, alkylsulfenyl, alkylcarbonyloxy, alkylthio, acylthio, aminosulfonyl, phosphorus-containing groups such as optionally substituted phosphono and optionally substituted phosphinyl, sulfinyl, sulfinylamino, sulfonyl, sulfonylamino, aryl, arylalkyl, arylalkyloxy, arylamino, Arylheteroalkyl, heteroaryl, heteroarylalkyl, heteroarylalkenyl, heteroarylalkynyl, heteroarylheteroalkyl, heteroarylamino, heteroaryloxy, arylalkenyl, arylalkyl, alkylaryl, alkylheteroaryl, aryloxy, arylsulfonyl, cyano, cyanate, isocyanate, —C(O)NH(alkyl), and —C(O)N(alkyl)₂.

“Acyl” means an R—C(═O)— group in which the R group may be an optionally substituted alkyl, optionally substituted cycloalkyl, optionally substituted heterocycloalkyl, optionally substituted aryl or optionally substituted heteroaryl group as defined herein. Examples of acyl include acetyl, benzoyl and amino acid derived aminoacyl. The group may be a terminal group or a bridging group. If the group is a terminal group it is bonded to the remainder of the molecule through the carbonyl carbon.

“Acylamino” means an R—C(═O)—NH— group in which the R group may be an alkyl, cycloalkyl, heterocycloalkyl, aryl or heteroaryl group as defined herein. The group may be a terminal group or a bridging group. If the group is a terminal group it is bonded to the remainder of the molecule through the nitrogen atom.

“Acyloxy” means an R—C(═O)—O— group in which the R group may be an alkyl, cycloalkyl, heterocycloalkyl, aryl or heteroaryl group as defined herein. The group may be a terminal group or a bridging group. If the group is a terminal group it is bonded to the remainder of the molecule through the oxygen atom.

“Alkenyl” as a group or part of a group denotes an aliphatic hydrocarbon group containing at least one carbon-carbon double bond and which may be straight or branched preferably having 2-12 carbon atoms, more preferably 2-10 carbon atoms, most preferably 2-6 carbon atoms, in the normal chain. The group may contain a plurality of double bonds in the normal chain and the orientation about each is independently E or Z. Exemplary alkenyl groups include, but are not limited to, ethenyl, propenyl, butenyl, pentenyl, hexenyl, heptenyl, octenyl and nonenyl. The group may be a terminal group or a bridging group.

“Alkenyloxy” refers to an alkenyl-O— group in which alkenyl is as defined herein. Preferred alkenyloxy groups are C₂-C₁₂ alkenyloxy groups. The group may be a terminal group or a bridging group. If the group is a terminal group it is bonded to the remainder of the molecule through the oxygen atom.

“Alkyl” as a group or part of a group refers to a straight or branched aliphatic hydrocarbon group, preferably a C₁-C₁₂ alkyl, more preferably a C₁-C₁₀ alkyl, most preferably C₁-C₆ unless otherwise noted. Examples of suitable straight and branched C₁-C₆ alkyl substituents include methyl, ethyl, n-propyl, 2-propyl, n-butyl, sec-butyl, t-butyl, hexyl, and the like. The group may be a terminal group or a bridging group.

“Alkylamino” includes both mono-alkylamino and dialkylamino, unless specified. “Mono-alkylamino” means a Alkyl-NH— group, in which alkyl is as defined herein.

“Dialkylamino” means a (alkyl)₂N— group, in which each alkyl may be the same or different and are each as defined herein for alkyl. The alkyl group is preferably a C₁-C₁₂ alkyl group. The group may be a terminal group or a bridging group. If the group is a terminal group it is bonded to the remainder of the molecule through the nitrogen atom.

“Alkylaminocarbonyl” refers to a group of the formula (Alkyl)ₓ(H)_{y}NC(═O)— in which alkyl is as defined herein, x is 1 or 2, and the sum of X+Y=2. The group may be a terminal group or a bridging group. If the group is a terminal group it is bonded to the remainder of the molecule through the carbonyl carbon.

“Alkylaryl” means an alkyl-aryl-- group in which the aryl and alkyl moieties are as defined herein. Preferred alkylaryl groups contain a C₁₋₁₂ alkyl moiety. The group may be a terminal group or a bridging group. If the group is a terminal group it is bonded to the remainder of the molecule through the aryl group.

“Alkyloxy” refers to an alkyl group as defined herein that is singularly bonded to oxygen. The group may be a terminal group or a bridging group. If the group is a terminal group, it is bonded to the remainder of the molecule through the alkyl group. “

“Alkyloxyalkyl” refers to an alkyloxy-alkyl- group in which the alkyloxy and alkyl moieties are as defined herein. The group may be a terminal group or a bridging group. If the group is a terminal group it is bonded to the remainder of the molecule through the alkyl group.

“Alkyloxyaryl” refers to an alkyloxy-aryl- group in which the alkyloxy and aryl moieties are as defined herein. The group may be a terminal group or a bridging group. If the group is a terminal group it is bonded to the remainder of the molecule through the aryl group.

“Alkyloxycarbonyl” refers to an alkyl-O—C(═O)— group in which alkyl is as defined herein. The alkyl group is preferably a C₁-C₁₂ alkyl group. Examples include, but are not limited to, methoxycarbonyl and ethoxycarbonyl. The group may be a terminal group or a bridging group. If the group is a terminal group it is bonded to the remainder of the molecule through the carbonyl carbon.

“Alkyloxycycloalkyl” refers to an alkyloxy-cycloalkyl- group in which the alkyloxy and cycloalkyl moieties are as defined herein. The group may be a terminal group or a bridging group. If the group is a terminal group it is bonded to the remainder of the molecule through the cycloalkyl group.

“Alkyloxyheteroaryl” refers to an alkyloxy-heteroaryl- group in which the alkyloxy and heteroaryl moieties are as defined herein. The group may be a terminal group or a bridging group. If the group is a terminal group it is bonded to the remainder of the molecule through the heteroaryl group.

“Alkyloxyheterocycloalkyl” refers to an alkyloxy-heterocycloalkyl- group in which the alkyloxy and heterocycloalkyl moieties are as defined herein. The group may be a terminal group or a bridging group. If the group is a terminal group it is bonded to the remainder of the molecule through the heterocycloalkyl group.

“Alkylsulfinyl” means an alkyl-S—(═O)— group in which alkyl is as defined herein. The alkyl group is preferably a C₁-C₁₂ alkyl group. Exemplary alkylsulfinyl groups include, but not limited to, methylsulfinyl and ethylsulfinyl. The group may be a terminal group or a bridging group. If the group is a terminal group it is bonded to the remainder of the molecule through the sulfur atom.

“Alkylsulfonyl” refers to an alkyl-S(═O)₂— group in which alkyl is as defined above. The alkyl group is preferably a C₁-C₁₂ alkyl group. Examples include, but not limited to methylsulfonyl and ethylsulfonyl. The group may be a terminal group or a bridging group. If the group is a terminal group it is bonded to the remainder of the molecule through the sulfur atom.

“Alkynyl” as a group or part of a group means an aliphatic hydrocarbon group containing a carbon-carbon triple bond and which may be straight or branched preferably having from 2-12 carbon atoms, more preferably 2-10 carbon atoms, more preferably 2-6 carbon atoms in the normal chain. Exemplary structures include, but are not limited to, ethynyl and propynyl. The group may be a terminal group or a bridging group.

“Alkynyloxy” refers to an alkynyl-O— group in which alkynyl is as defined herein. Preferred alkynyloxy groups are C₂-C₁₂ alkynyloxy groups. The group may be a terminal group or a bridging group. If the group is a terminal group it is bonded to the remainder of the molecule through the oxygen atom.

“Amino” refers to groups of the form —NRₐR_{b} wherein Rₐ and R_{b} are individually selected from the group including but not limited to hydrogen, optionally substituted alkyl, optionally substituted alkenyl, optionally substituted alkynyl, and optionally substituted aryl groups.

“Aminoalkyl” means an NH₂-alkyl- group in which the alkyl group is as defined herein. The group may be a terminal group or a bridging group. If the group is a terminal group it is bonded to the remainder of the molecule through the alkyl group.

“Aminosulfonyl” means an NH₂—S(═O)₂— group. The group may be a terminal group or a bridging group. If the group is a terminal group it is bonded to the remainder of the molecule through the sulfur atom.

“Aryl” as a group or part of a group denotes (i) an optionally substituted monocyclic, or fused polycyclic, aromatic carbocycle (ring structure having ring atoms that are all carbon) preferably having from 5 to 12 atoms per ring. Examples of aryl groups include phenyl, naphthyl, and the like; (ii) an optionally substituted partially saturated bicyclic aromatic carbocyclic moiety in which a phenyl and a C₅₋₇ cycloalkyl or C₅₋₇ cycloalkenyl group are fused together to form a cyclic structure, such as tetrahydronaphthyl, indenyl or indanyl. The group may be a terminal group or a bridging group. Typically an aryl group is a C₆-C₁₈ aryl group.

“Arylalkenyl” means an aryl-alkenyl- group in which the aryl and alkenyl are as defined herein. Exemplary arylalkenyl groups include phenylallyl. The group may be a terminal group or a bridging group. If the group is a terminal group it is bonded to the remainder of the molecule through the alkenyl group.

“Arylalkyl” means an aryl-alkyl- group in which the aryl and alkyl moieties are as defined herein. Preferred arylalkyl groups contain a C₁₋₁₂ alkyl moiety. Exemplary arylalkyl groups include benzyl, phenethyl, 1-naphthalenemethyl and 2-naphthalenemethyl. The group may be a terminal group or a bridging group. If the group is a terminal group it is bonded to the remainder of the molecule through the alkyl group.

“Arylalkyloxy” refers to an aryl-alkyl-O— group in which the alkyl and aryl are as defined herein. The group may be a terminal group or a bridging group. If the group is a terminal group it is bonded to the remainder of the molecule through the oxygen atom.

“Arylamino” includes both mono-arylamino and di-arylamino unless specified. Mono-arylamino means a group of formula arylNH—, in which aryl is as defined herein. di-arylamino means a group of formula (aryl)₂N— where each aryl may be the same or different and are each as defined herein for aryl. The group may be a terminal group or a bridging group. If the group is a terminal group it is bonded to the remainder of the molecule through the nitrogen atom.

“Arylheteroalkyl” means an aryl-heteroalkyl- group in which the aryl and heteroalkyl moieties are as defined herein. The group may be a terminal group or a bridging group. If the group is a terminal group it is bonded to the remainder of the molecule through the heteroalkyl group.

“Aryloxy” refers to an aryl-O— group in which the aryl is as defined herein. Preferably the aryloxy is a C₅-C₁₈aryloxy, more preferably a C₅-C₁₀aryloxy. The group may be a terminal group or a bridging group. If the group is a terminal group it is bonded to the remainder of the molecule through the oxygen atom.

“Arylsulfonyl” means an aryl-S(═O)₂— group in which the aryl group is as defined herein. The group may be a terminal group or a bridging group. If the group is a terminal group it is bonded to the remainder of the molecule through the sulfur atom.

A “bond” is a linkage between atoms in a compound or molecule. The bond may be a single bond, a double bond, or a triple bond.

“Cycloalkenyl” means a non-aromatic monocyclic or multicyclic ring system containing at least one carbon-carbon double bond and preferably having from 5-12 carbon atoms per ring. Exemplary monocyclic cycloalkenyl rings include cyclopentenyl, cyclohexenyl or cycloheptenyl. The cycloalkenyl group may be substituted by one or more substituent groups. A cycloalkenyl group typically is a C₅-C₁₂ alkenyl group. The group may be a terminal group or a bridging group.

“Cycloalkyl” refers to a saturated monocyclic or fused or spiro polycyclic, carbocycle preferably containing from 3 to 12 carbons per ring, such as cyclopropyl, cyclobutyl, cyclopentyl, cyclohexyl and the like, unless otherwise specified. It includes monocyclic systems such as cyclopropyl and cyclohexyl, bicyclic systems such as decalin, and polycyclic systems such as adamantane. A cycloalkyl group typically is a C₃-C₁₂ alkyl group. The group may be a terminal group or a bridging group.

“Cycloalkylalkyl” means a cycloalkyl-alkyl- group in which the cycloalkyl and alkyl moieties are as defined herein. Exemplary monocycloalkylalkyl groups include cyclopropylmethyl, cyclopentylmethyl, cyclohexylmethyl and cycloheptylmethyl. The group may be a terminal group or a bridging group. If the group is a terminal group it is bonded to the remainder of the molecule through the alkyl group.

“Cycloalkylalkenyl” means a cycloalkyl-alkenyl- group in which the cycloalkyl and alkenyl moieties are as defined herein. The group may be a terminal group or a bridging group. If the group is a terminal group it is bonded to the remainder of the molecule through the alkenyl group.

“Cycloalkylheteroalkyl” means a cycloalkyl-heteroalkyl- group in which the cycloalkyl and heteroalkyl moieties are as defined herein. The group may be a terminal group or a bridging group. If the group is a terminal group it is bonded to the remainder of the molecule through the heteroalkyl group.

“Cycloalkyloxy” refers to a cycloalkyl-O— group in which cycloalkyl is as defined herein. Preferably the cycloalkyloxy is a C₃-C₁₂cycloalkyloxy. Examples include, but are not limited to, cyclopropanoxy and cyclobutanoxy. The group may be a terminal group or a bridging group. If the group is a terminal group it is bonded to the remainder of the molecule through the oxygen atom.

“Cycloalkenyloxy” refers to a cycloalkenyl-O— group in which the cycloalkenyl is as defined herein. Preferably the cycloalkenyloxy is a C₃-C₁₂cycloalkenyloxy. The group may be a terminal group or a bridging group. If the group is a terminal group it is bonded to the remainder of the molecule through the oxygen atom.

“Cycloamino” refers to a saturated monocyclic, bicyclic, or polycyclic ring containing at least one nitrogen in at least one ring. Each ring is preferably from 3 to 10 membered, more preferably 4 to 7 membered. The group may be a terminal group or a bridging group. If the group is a terminal group it is bonded to the remainder of the molecule through the nitrogen atom.

“Haloalkyl” refers to an alkyl group as defined herein in which one or more of the hydrogen atoms has been replaced with a halogen atom selected from the group consisting of fluorine, chlorine, bromine and iodine. A haloalkyl group typically has the formula CₙH₍₂ₙ₊₁₋ₘ₎Xₘ wherein each X is independently selected from the group consisting of F, Cl, Br and I. In groups of this type n is typically from 1 to 10, more preferably from 1 to 6, most preferably 1 to 3. m is typically 1 to 6, more preferably 1 to 3. Examples of haloalkyl include fluoromethyl, difluoromethyl and trifluoromethyl.

“Haloalkenyl” refers to an alkenyl group as defined herein in which one or more of the hydrogen atoms has been replaced with a halogen atom independently selected from the group consisting of F, Cl, Br and I.

“Haloalkynyl” refers to an alkynyl group as defined herein in which one or more of the hydrogen atoms has been replaced with a halogen atom independently selected from the group consisting of F, Cl, Br and I.

“Halogen” represents chlorine, fluorine, bromine or iodine.

“Heteroalkyl” refers to a straight- or branched-chain alkyl group preferably having from 2 to 12 carbons, more preferably 2 to 6 carbons in the chain, one or more of which has been replaced by a heteroatom selected from S, O, P and N. Exemplary heteroalkyls include alkyl ethers, secondary and tertiary alkyl amines, amides, alkyl sulfides, and the like. Examples of heteroalkyl also include hydroxyC₁-C₆alkyl, C₁-C₆alkyloxyC₁-C₆alkyl, aminoC₁-C₆alkyl, C₁-C₆alkylaminoC₁-C₆alkyl, and di(C₁-C₆alkyl)aminoC₁-C₆alkyl. The group may be a terminal group or a bridging group.

“Heteroalkenyl” refers to a straight- or branched-chain alkenyl group preferably having from 2 to 12 carbons, more preferably 2 to 6 carbons in the chain, one or more of which has been replaced by a heteroatom selected from S, O, P and N. Exemplary heteroalkenyls include alkenyl ethers, secondary and tertiary alkenyl amines, amides, alkenyl sulfides, and the like. Examples of heteroalkenyl also include hydroxyC₁-C₆alkenyl, C₁-C₆alkyloxyC₁-C₆alkenyl, aminoC₁-C₆alkenyl, C₁-C₆alkylaminoC₁-C₆alkenyl, and di(C₁-C₆alkyl)aminoC₁-C₆alkenyl. The group may be a terminal group or a bridging group.

“Heteroalkynyl” refers to a straight- or branched-chain alkenyl group preferably having from 2 to 12 carbons, more preferably 2 to 6 carbons in the chain, one or more of which has been replaced by a heteroatom selected from S, O, P and N. Exemplary heteroalkynyls include alkynyl ethers, secondary and tertiary alkynyl amines, amides, alkynyl sulfides, and the like. Examples of heteroalkynyl also include hydroxyC₁-C₆alkynyl, C₁-C₆alkyloxyC₁-C₆alkynyl, aminoC₁-C₆alkynyl, C₁-C₆alkylaminoC₁-C₆alkynyl, and di(C₁-C₆alkyl)aminoC₁-C₆alkynyl. The group may be a terminal group or a bridging group.

“Heteroalkyloxy” refers to an heteroalkyl-O— group in which heteroalkyl is as defined herein. Preferably the heteroalkyloxy is a C₁-C₁₂heteroalkyloxy. The group may be a terminal group or a bridging group.

“Heteroaryl” either alone or part of a group refers to groups containing an aromatic ring (preferably a 5 or 6 membered aromatic ring) having one or more heteroatoms as ring atoms in the aromatic ring with the remainder of the ring atoms being carbon atoms. Suitable heteroatoms include nitrogen, oxygen and sulphur. Examples of heteroaryl include thiophene, benzothiophene, benzofuran, benzimidazole, benzoxazole, benzothiazole, benzisothiazole, naphtho[2,3-b]thiophene, furan, isoindolizine, xantholene, phenoxatine, pyrrole, imidazole, pyrazole, pyridine, pyrazine, pyrimidine, pyridazine, tetrazole, indole, isoindole, 1H-indazole, purine, quinoline, isoquinoline, phthalazine, naphthyridine, quinoxaline, cinnoline, carbazole, phenanthridine, acridine, phenazine, thiazole, isothiazole, phenothiazine, oxazole, isooxazole, furazane, phenoxazine, 2-, 3- or 4-pyridyl, 2-, 3-, 4-, 5-, or 8-quinolyl, 1-, 3-, 4-, or 5-isoquinolinyl 1-, 2-, or 3-indolyl, and 2-, or 3-thienyl. A heteroaryl group is typically a C₁-C₁₈ heteroaryl group. A heteroaryl group may comprise 3 to 8 ring atoms. A heteroaryl group may comprise 1 to 3 heteroatoms independently selected from the group consisting of N, O and S. The group may be a terminal group or a bridging group.

“Heteroarylalkyl” means a heteroaryl-alkyl group in which the heteroaryl and alkyl moieties are as defined herein. Preferred heteroarylalkyl groups contain a lower alkyl moiety. Exemplary heteroarylalkyl groups include pyridylmethyl. The group may be a terminal group or a bridging group. If the group is a terminal group it is bonded to the remainder of the molecule through the alkyl group.

“Heteroarylalkenyl” means a heteroaryl-alkenyl- group in which the heteroaryl and alkenyl moieties are as defined herein. The group may be a terminal group or a bridging group. If the group is a terminal group it is bonded to the remainder of the molecule through the alkenyl group.

“Heteroarylheteroalkyl” means a heteroaryl-heteroalkyl- group in which the heteroaryl and heteroalkyl moieties are as defined herein. The group may be a terminal group or a bridging group. If the group is a terminal group it is bonded to the remainder of the molecule through the heteroalkyl group.

“Heteroarylamino” refers to groups containing an aromatic ring (preferably 5 or 6 membered aromatic ring) having at least one nitrogen and at least another heteroatom as ring atoms in the aromatic ring, preferably from 1 to 3 heteroatoms in at least one ring. Suitable heteroatoms include nitrogen, oxygen and sulphur. Arylamino and aryl is as defined herein. The group may be a terminal group or a bridging group. If the group is a terminal group it is bonded to the remainder of the molecule through the nitrogen atom.

“Heteroaryloxy” refers to a heteroaryl-O— group in which the heteroaryl is as defined herein. Preferably the heteroaryloxy is a C₁-C₁₈heteroaryloxy. The group may be a terminal group or a bridging group. If the group is a terminal group it is bonded to the remainder of the molecule through the oxygen atom.

“Heterocyclic” refers to saturated, partially unsaturated or fully unsaturated monocyclic, bicyclic or polycyclic ring system containing at least one heteroatom selected from the group consisting of nitrogen, sulfur and oxygen as a ring atom. Examples of heterocyclic moieties include heterocycloalkyl, heterocycloalkenyl and heteroaryl.

“Heterocycloalkenyl” refers to a heterocycloalkyl as defined herein but containing at least one double bond. A heterocycloalkenyl group typically is a C₁-C₁₂ heterocycloalkenyl group. The group may be a terminal group or a bridging group.

“Heterocycloalkyl” refers to a saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane, and 1,4-oxathiapane. A heterocycloalkyl group typically is a C₁-C₁₂ heterocycloalkyl group. A heterocycloalkyl group may comprise 3 to 8 ring atoms. A heterocycloalkyl group may comprise 1 to 3 heteroatoms independently selected from the group consisting of N, O and S. The group may be a terminal group or a bridging group.

“Heterocycloalkylalkyl” refers to a heterocycloalkyl-alkyl- group in which the heterocycloalkyl and alkyl moieties are as defined herein. Exemplary heterocycloalkylalkyl groups include (2-tetrahydrofuryl)methyl, (2-tetrahydrothiofuranyl) methyl. The group may be a terminal group or a bridging group. If the group is a terminal group it is bonded to the remainder of the molecule through the alkyl group.

“Heterocycloalkylalkenyl” refers to a heterocycloalkyl-alkenyl- group in which the heterocycloalkyl and alkenyl moieties are as defined herein. The group may be a terminal group or a bridging group. If the group is a terminal group it is bonded to the remainder of the molecule through the alkenyl group.

“Heterocycloalkylheteroalkyl” means a heterocycloalkyl-heteroalkyl- group in which the heterocycloalkyl and heteroalkyl moieties are as defined herein. The group may be a terminal group or a bridging group. If the group is a terminal group it is bonded to the remainder of the molecule through the heteroalkyl group.

“Heterocycloalkyloxy” refers to a heterocycloalkyl-O— group in which the heterocycloalkyl is as defined herein. Preferably the heterocycloalkyloxy is a C₁-C₆heterocycloalkyloxy. The group may be a terminal group or a bridging group. If the group is a terminal group it is bonded to the remainder of the molecule through the oxygen atom.

“Heterocycloalkenyloxy” refers to a heterocycloalkenyl-O— group in which heterocycloalkenyl is as defined herein. Preferably the Heterocycloalkenyloxy is a C₁-C₆ Heterocycloalkenyloxy. The group may be a terminal group or a bridging group. If the group is a terminal group it is bonded to the remainder of the molecule through the oxygen atom.

“Heterocycloamino” refers to a saturated monocyclic, bicyclic, or polycyclic ring containing at least one nitrogen and at least another heteroatom selected from nitrogen, sulfur, oxygen, preferably from 1 to 3 heteroatoms in at least one ring. Each ring is preferably from 3 to 10 membered, more preferably 4 to 7 membered. The group may be a terminal group or a bridging group. If the group is a terminal group it is bonded to the remainder of the molecule through the nitrogen atom.

“Hydroxyalkyl” refers to an alkyl group as defined herein in which one or more of the hydrogen atoms has been replaced with an OH group. A hydroxyalkyl group typically has the formula CₙH₍₂ₙ₊₁₋ₓ₎(OH)ₓ. In groups of this type n is typically from 1 to 10, more preferably from 1 to 6, most preferably from 1 to 3. x is typically from 1 to 6, more preferably from 1 to 4.

“Sulfinyl” means an R—S(═O)— group in which the R group may be OH, alkyl, cycloalkyl, heterocycloalkyl; aryl or heteroaryl group as defined herein. The group may be a terminal group or a bridging group. If the group is a terminal group it is bonded to the remainder of the molecule through the sulfur atom.

“Sulfinylamino” means an R—S(═O)—NH— group in which the R group may be OH, alkyl, cycloalkyl, heterocycloalkyl; aryl or heteroaryl group as defined herein. The group may be a terminal group or a bridging group. If the group is a terminal group it is bonded to the remainder of the molecule through the nitrogen atom.

“Sulfonyl” means an R—S(═O)₂— group in which the R group may be OH, alkyl, cycloalkyl, heterocycloalkyl; aryl or heteroaryl group as defined herein. The group may be a terminal group or a bridging group. If the group is a terminal group it is bonded to the remainder of the molecule through the sulfur atom.

“Sulfonylamino” means an R—S(═O)₂—NH— group. The group may be a terminal group or a bridging group. If the group is a terminal group it is bonded to the remainder of the molecule through the nitrogen atom.

The term “pharmaceutically acceptable excipient” may refer to a non-toxic carrier, adjuvant, or vehicle that does not destroy the pharmacological activity of the compound with which it is formulated. Pharmaceutically acceptable carriers, adjuvants or vehicles that may be used in the compositions of this disclosure may include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol or wool fat.

The term “biguanide derivative”, for the purposes of this disclosure, refers to any compound having the following formula (A):

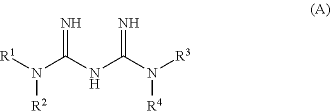

wherein R¹, R², R³ and R⁴ for each occurrence is independently selected from hydrogen, optionally substituted alkyl, optionally substituted alkenyl or optionally substituted alkynyl.

Some of the compounds of the disclosed embodiments may exist as single stereoisomers, racemates, and/or mixtures of enantiomers and/or diastereomers. All such single stereoisomers, racemates and mixtures thereof, are intended to be within the scope of the subject matter described and claimed.

Further, it is possible that compounds of the invention may contain more than one asymmetric carbon atom. In those compounds, the use of a solid line to depict bonds to asymmetric carbon atoms is meant to indicate that all possible stereoisomers are meant to be included. The use of a solid line to depict bonds to one or more asymmetric carbon atoms in a compound of the invention and the use of a solid or dotted wedge to depict bonds to other asymmetric carbon atoms in the same compound is meant to indicate that a mixture of diastereomers is present.

It is understood that included in the family of disclosed compounds are isomeric forms including diastereoisomers, enantiomers, tautomers, and geometrical isomers in “E” or “Z” configurational isomer or a mixture of E and Z isomers. It is also understood that some isomeric forms such as diastereomers, enantiomers, and geometrical isomers can be separated by physical and/or chemical methods and by those skilled in the art.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### DETAILED DESCRIPTION

Metformin is a first-line treatment for Type II diabetes and listed as one of World Health Organization (WHO) essential medicines. Phenformin is a more lipophilic analogue of metformin which was also used as antidiabetic drug, but later removed from the market due to severe side effects. The anticancer role of metformin and phenformin was recognized after retrospective epidemiological analyses, which revealed that they significantly reduced cancer risks in diabetic patients. Both biguanides were also repeatedly shown to enhance antiproliferative effects of other drugs, including cisplatin, 2-deoxyglucose, doxorubicin, tamoxifen in a synergistic manner both in vitro and in vivo. However, further preclinical development of metformin and phenformin as anticancer drugs is hindered by their poor cellular uptake and side effects associated with high dosages.

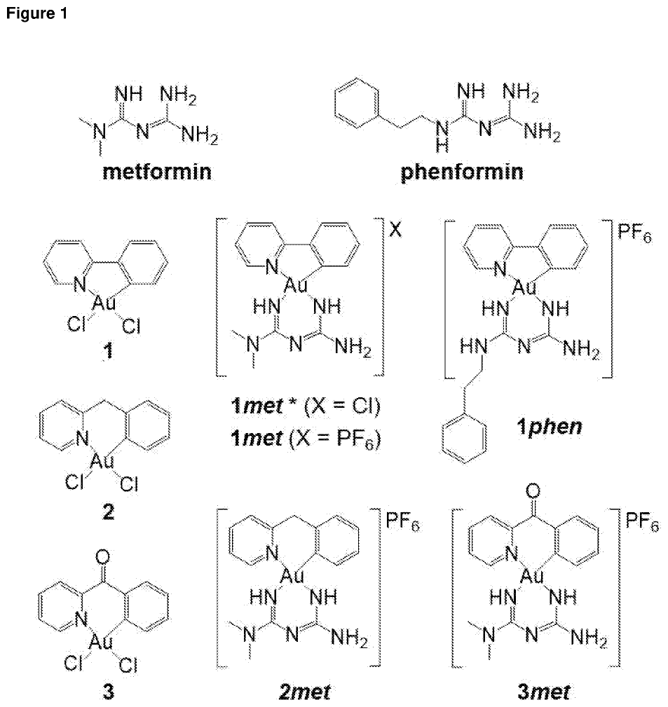

Here, five novel organometallic Au^{III}-metformin and phenformin complexes (1met*1-3met, 1phen and 1met*) were developed (FIG. 1), where metformin and phenformin, for the first time, were chemically conjugated to another active pharmacophore. The design of novel complexes was based on the following parameters: 1) the lipophilic cyclometalated fragments featuring bidentate ligands as shown in FIG. 1 were aimed to enhance the intracellular delivery of metformin and phenformin; 2) the chemically labile Au^{III} fragments were expected to release metformin and phenformin once inside the cells; 3) since metformin and Au^{III} pharmacophores were both reported to induce cellular metabolic perturbations, they were expected to act synergistically leading to a deadly metabolic catastrophe in cancer cells.

There is provided a compound of formula (I):

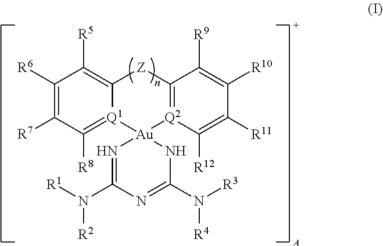

wherein,
n may be 0 or 1;
Z may be C, N, O, S, Si, C═O or C═S;
Q¹ and Q² for each occurrence may be independently selected from C, P or N, wherein at least one of Q¹ or Q² is N;
R¹, R², R³ and R⁴ for each occurrence may be independently selected from the group consisting of hydrogen, optionally substituted alkyl, optionally substituted alkenyl, and optionally substituted alkynyl, provided that at least one of R¹, R², R³ or R⁴ is an optionally substituted alkyl;
R⁵, R⁶, R⁷, R⁸, R⁹, R¹⁰, R¹¹ and R¹² for each occurrence may be independently selected from the group consisting of hydrogen, —X, —OR′, —SR′, —P(R′)₂, —C(═O)OR′, C(═O)R′, —COX, —CX₃, —NO₂, —SO₃H, —SO₂R′, —N═O, optionally substituted alkyl, optionally substituted amino, optionally substituted alkyloxy, optionally substituted cycloalkyl, optionally substituted heterocycloalkyl, optionally substituted aryl, and optionally substituted heteroaryl, wherein R′ is independently hydrogen or an optionally substituted alkyl and X is halogen; and
A may be tosylate (OTs⁻), mesylate (OMs⁻), BF₄⁻, NO₃⁻, Cl⁻, Br, triflate (OTf⁻), ClO₄⁻, bistriflimide (TFSI⁻), BPH₄⁻, BPh₄^{F-}, tetrakis(1-imidazolyl)borate (Blm₄⁻), tetrakis(2-thienyl)borate (BTH₄⁻), HPO₄²⁻ or PF₆.

Q¹ may be N and Q² may be C or Q¹ may be C and Q² may be N.

At least one of R⁵, R⁶, R⁷, R⁸, R⁹, R¹⁰, R¹¹ and R¹² may be independently an optionally substituted alkyl.

R⁷ may be an optionally substituted alkyl, and R⁵, R⁶, R⁸, R⁹, R¹⁰, R¹¹ and R¹² may be hydrogen. R⁷ may be butyl.

R⁵, R⁶, R⁷, R⁸, R⁹, R¹⁰, R¹¹ and R¹² may be hydrogen.

R³ and R⁴ may be hydrogen.

R¹ and R² may independently be hydrogen or optionally substituted alkyl. R¹ and R² may independently be selected from the group consisting of methyl, ethyl, n-propyl, isopropyl, n-butyl, ter-butyl, sec-butyl, isobutyl, n-pentyl, tert-pentyl, neopentyl, isopentyl, sec-pentyl, 3-pentyl, sec-isopentyl and active pentyl.

R¹ may be hydrogen and R² may be methyl.

R¹ and R² may be methyl.

R¹ may be hydrogen and R² may be a substituted alkyl.

R² may be an arylalkyl.

R² may have the following formula (IIa):

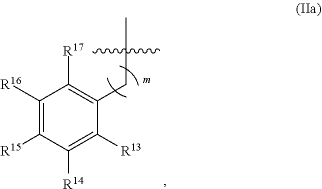

wherein R¹³, R¹⁴, R¹⁵, R¹⁶ and R¹⁷ for each occurrence is independently selected from hydrogen, optionally substituted alkyl, optionally substituted alkenyl or optionally substituted alkynyl and wherein m may be an integer from 1 to 10.

R¹³, R¹⁴, R¹⁵, R¹⁶ and R¹⁷ may be hydrogen.

R² may have the following formula (IIb):

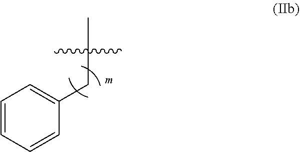

wherein m may be an integer from 1 to 10.

m may be 1, 2, 3, 4, 5, 6, 7, 8, 9 or 10.

m may be 2.

n may be 0.

n may be 1 and Z may be C or C═O.

The compound may have the following formula (IIIa), (IIIb), (IIIc), (IIId) or (IIIe):

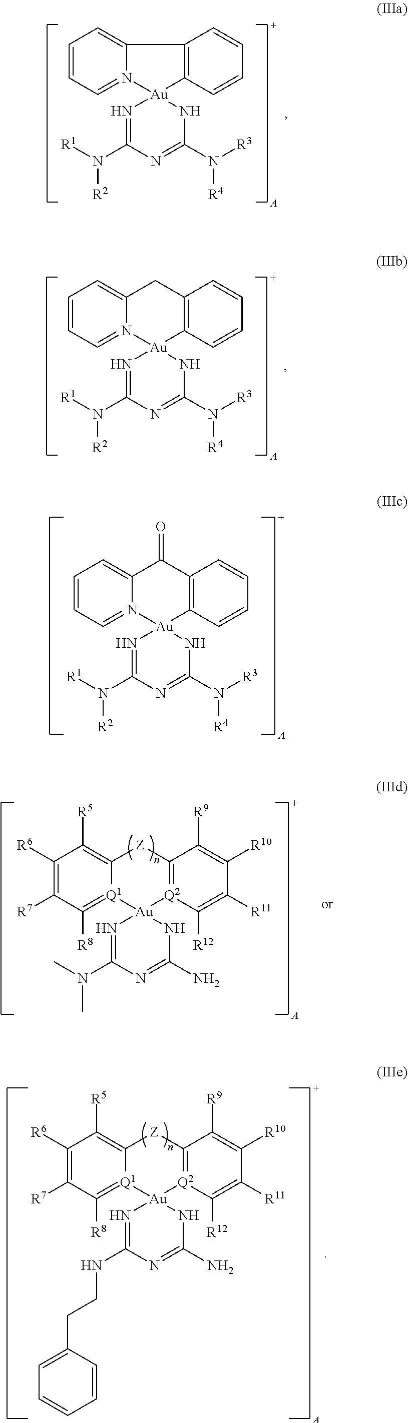

The compound may have the following structure:

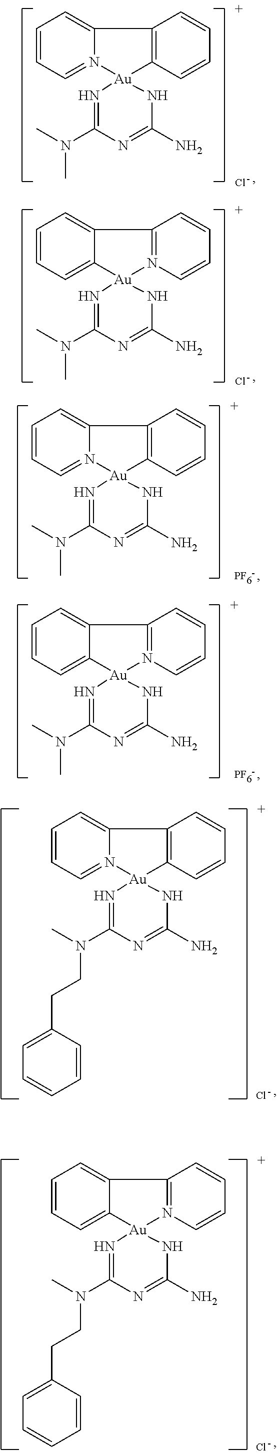

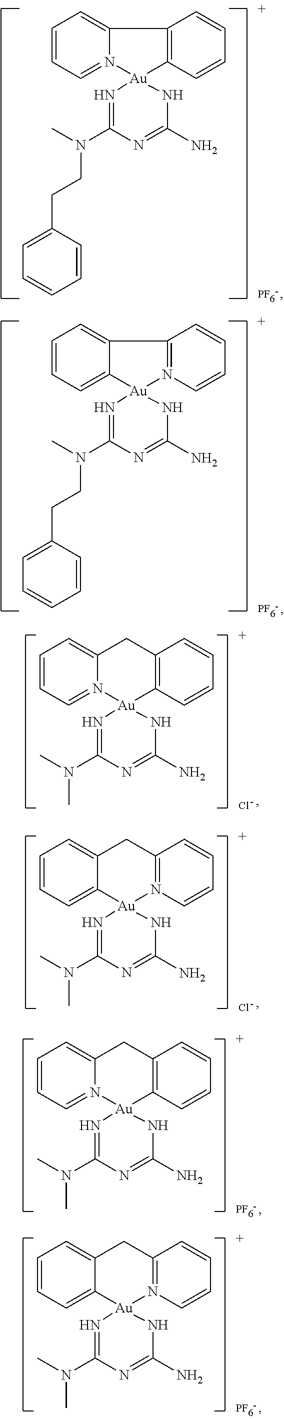

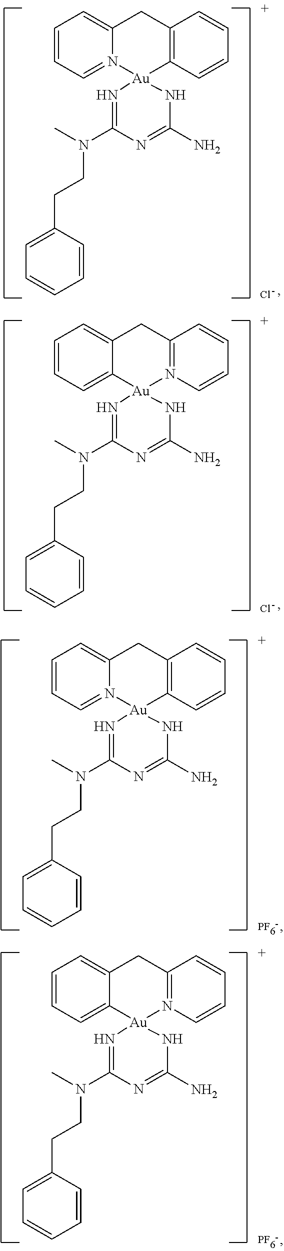

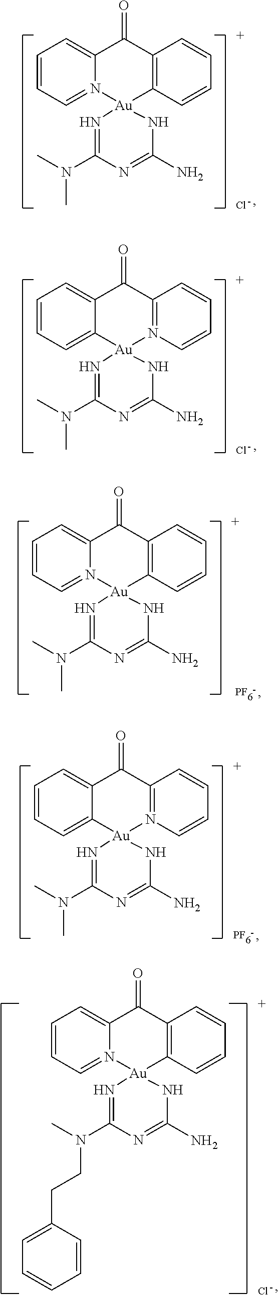

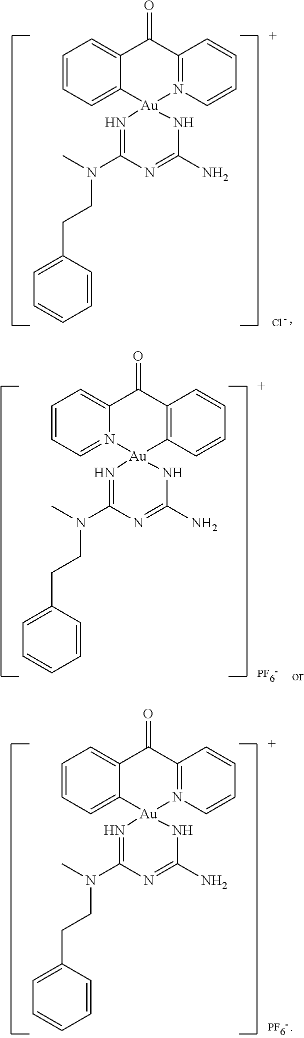

The compound may have the following structure:

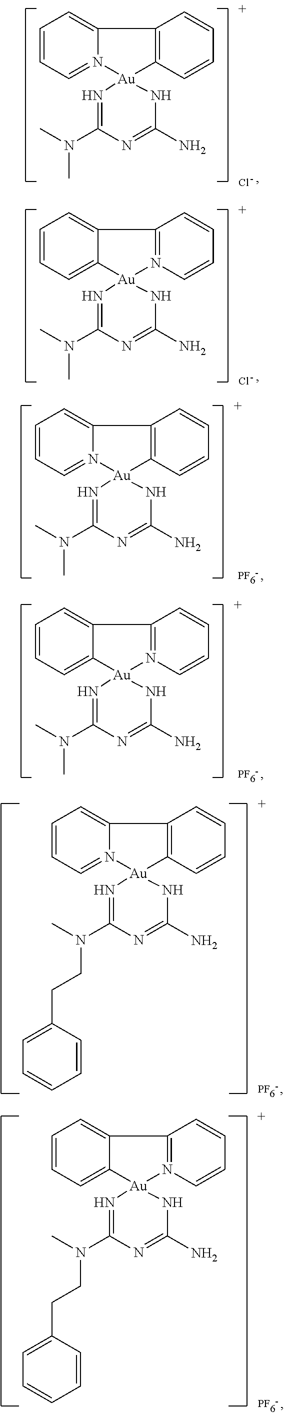

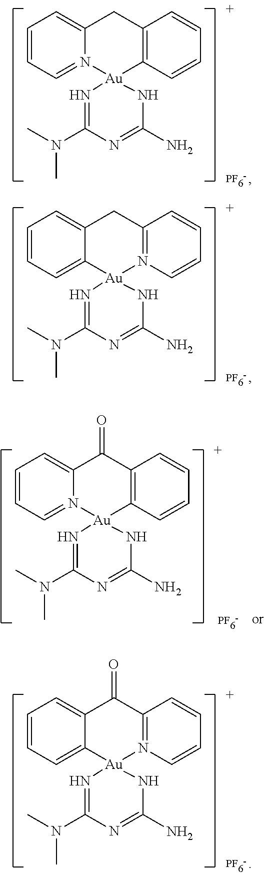

There is provided a method for preparing a compound as defined above, the method comprising the step of contacting:
a compound having the following formula (IV):

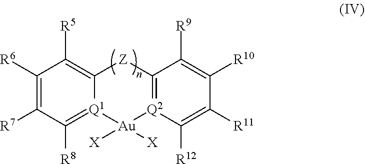

with a compound having the following formula (V):

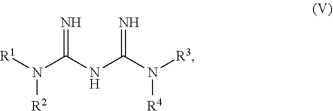

wherein
n may be 0 or 1;
Z may be C, N, O, S, Si, C═O or C═S,
Q¹ and Q² for each occurrence may be independently selected from C, P or N, wherein at least one of Q¹ or Q² is N;
R¹, R², R³ and R⁴ for each occurrence may be independently selected from the group consisting of hydrogen, optionally substituted alkyl, and optionally substituted alkenyl, optionally substituted alkynyl, provided that at least one of R¹, R², R³ or R⁴ is an optionally substituted alkyl;
R⁵, R⁶, R⁷, R⁸, R⁹, R¹⁰, R¹¹ and R¹² for each occurrence may be independently selected from the group consisting of hydrogen, —X, —OR′, —SR′, —P(R′)₂, —C(═O)OR′, C(═O)R′, —COX, —CX₃, —NO₂, —SO₃H, —SO₂R′, —N═O, optionally substituted alkyl, optionally substituted amino, optionally substituted alkyloxy, optionally substituted cycloalkyl, optionally substituted heterocycloalkyl, optionally substituted aryl, and optionally substituted heteroaryl, wherein R′ is independently hydrogen or an optionally substituted alkyl and X is halogen.

There is provided a pharmaceutical composition comprising a compound as defined above and a pharmaceutically acceptable excipient.

There is provided a method for treating cancer comprising the step of administering to a subject in need thereof a compound as defined above or a pharmaceutical composition as defined above.

There is provided a compound as defined above or a pharmaceutical composition as defined above for use as a medicament.

There is provided a compound as defined above or a pharmaceutical composition as defined above for use in the treatment of cancer.

There is provided the use of a compound as defined above or a pharmaceutical composition as defined above in the manufacture of a medicament for the treatment of cancer.

The cancer may be selected from the group consisting of carcinoma, sarcoma, lymphoma, leukemia, germ cell tumor, blastoma, brain cancer, medulloblastoma, glioma, head and neck cancer, oral cancer, laryngeal cancer, nasopharynx cancer, thyroid cancer, myeloid neoplasm, lung cancer, non-small cell lung cancer, small cell lung cancer, mesothelioma, liver cancer, hepatocellular carcinoma, lymphoid neoplasm, bone cancer, ewing sarcoma, osteosarcoma, skeletal cancer, muscle cancer, rhabdomyosarcoma, skin cancer, basal cell carcinoma, squamous cell carcinoma, connective tissue cancer, fibrosarcoma, cartilage cancer, chondrosarcoma, nerve tissue cancer, neuroblastoma, gastric cancer, esophageal cancer, stomach cancer, pancreatic cancer, kidney cancer, bladder cancer, colorectal cancer, prostate cancer, breast cancer, triple-negative breast cancer, testicular cancer, ovarian cancer, uterine cancer, fallopian tube cancer, cervical cancer and metastatic cancer.

The cancer may be selected from the group consisting of medulloblastoma, ewing sarcoma, osteosarcoma, rhabdomyosarcoma, fibrosarcoma connective, chondrosarcoma, neuroblastoma, esophageal cancer, head and neck cancer, gastric cancer, pancreatic cancer, glioma, bladder cancer, kidney cancer, prostate cancer, melanoma,-mesothelioma, hepatocellular carcinoma, triple-negative breast cancer, colon cancer and metastatic cancer.

The cancer may be breast cancer. The cancer may be triple negative breast cancer.

The compound as defined above or the pharmaceutical composition as defined above may also be useful in treating diseases, disorders or conditions mediated by the immune system.

In accordance with the present invention, when used for the treatment of cancer, compound(s) of the invention may be administered alone. Alternatively, the compounds may be administered as a pharmaceutical, veterinarial, agricultural, or industrial formulation which comprises at least one compound according to the invention. The compound(s) may also be present as suitable salts, including pharmaceutically acceptable salts.

Combinations of active agents, including compounds of the invention, may be synergistic.

By pharmaceutically acceptable salt it is meant those salts which, within the scope of sound medical judgement, are suitable for use in contact with the tissues of humans and lower animals without undue toxicity, irritation, allergic response and the like, and are commensurate with a reasonable benefit/risk ratio. Pharmaceutically acceptable salts are well known in the art.

For instance, suitable pharmaceutically acceptable salts of compounds according to the present invention may be prepared by mixing a pharmaceutically acceptable acid such as hydrochloric acid, sulfuric acid, methanesulfonic acid, succinic acid, fumaric acid, maleic acid, benzoic acid, phosphoric acid, acetic acid, oxalic acid, carbonic acid, tartaric acid, or citric acid with the compounds of the invention. Suitable pharmaceutically acceptable salts of the compounds of the present invention therefore include acid addition salts.

S. M. Berge et al. describe pharmaceutically acceptable salts in detail in J. Pharmaceutical Sciences, 1977, 66:1-19. The salts can be prepared in situ during the final isolation and purification of the compounds of the invention, or separately by reacting the free base function with a suitable organic acid. Representative acid addition salts include acetate, adipate, alginate, ascorbate, asparate, benzenesulfonate, benzoate, bisulfate, borate, butyrate, camphorate, camphorsulfonate, citrate, digluconate, cyclopentanepropionate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium potassium, calcium, magnesium, and the like, as well as non-toxic ammonium, quaternary ammonium, and amine cations, including, but not limited to ammonium, tetramethylammonium, tetraethylammonium, methylamine, dimethylamine, trimethylamine, triethylamine, ethylamine, triethanolamine and the like.

Convenient modes of administration include injection (subcutaneous, intravenous, etc.), oral administration, inhalation, transdermal application, topical creams or gels or powders, or rectal administration. Depending on the route of administration, the formulation and/or compound may be coated with a material to protect the compound from the action of enzymes, acids and other natural conditions which may inactivate the therapeutic activity of the compound. The compound may also be administered parenterally or intraperitoneally.

Dispersions of the compounds according to the invention may also be prepared in glycerol, liquid polyethylene glycols, and mixtures thereof and in oils. Under ordinary conditions of storage and use, pharmaceutical preparations may contain a preservative to prevent the growth of microorganisms.

Pharmaceutical compositions suitable for injection include sterile aqueous solutions (where water soluble) or dispersions and sterile powders for the extemporaneous preparation of sterile injectable solutions or dispersions. Ideally, the composition is stable under the conditions of manufacture and storage and may include a preservative to stabilise the composition against the contaminating action of microorganisms such as bacteria and fungi.

In one embodiment of the invention, the compound(s) of the invention may be administered orally, for example, with an inert diluent or an assimilable edible carrier. The compound(s) and other ingredients may also be enclosed in a hard or soft shell gelatin capsule, compressed into tablets, or incorporated directly into an individual's diet. For oral therapeutic administration, the compound(s) may be incorporated with excipients and used in the form of ingestible tablets, buccal tablets, troches, capsules, elixirs, suspensions, syrups, wafers, and the like. Suitably, such compositions and preparations may contain at least 1% by weight of active compound. The percentage of the compound(s) in pharmaceutical compositions and preparations may, of course, be varied and, for example, may conveniently range from about 2% to about 90%, about 5% to about 80%, about 10% to about 75%, about 15% to about 65%; about 20% to about 60%, about 25% to about 50%, about 30% to about 45%, or about 35% to about 45%, of the weight of the dosage unit. The amount of compound in therapeutically useful compositions is such that a suitable dosage will be obtained.

The language “pharmaceutically acceptable carrier” is intended to include solvents, dispersion media, coatings, anti-bacterial and anti-fungal agents, isotonic and absorption delaying agents, and the like. The use of such media and agents for pharmaceutically active substances is well known in the art. Except insofar as any conventional media or agent is incompatible with the compound, use thereof in the therapeutic compositions and methods of treatment and prophylaxis is contemplated. Supplementary active compounds may also be incorporated into the compositions according to the present invention. It is especially advantageous to formulate parenteral compositions in dosage unit form for ease of administration and uniformity of dosage. “Dosage unit form” as used herein refers to physically discrete units suited as unitary dosages for the individual to be treated; each unit containing a predetermined quantity of compound(s) is calculated to produce the desired therapeutic effect in association with the required pharmaceutical carrier. The compound(s) may be formulated for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

In one embodiment, the carrier may be an orally administrable carrier.

Another form of a pharmaceutical composition is a dosage form formulated as enterically coated granules, tablets or capsules suitable for oral administration.

Also included in the scope of this invention are delayed release formulations.

In one embodiment, the compound may be administered by injection. In the case of injectable solutions, the carrier can be a solvent or dispersion medium containing, for example, water, ethanol, polyol (for example, glycerol, propylene glycol, and liquid polyetheylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by including various anti-bacterial and/or anti-fungal agents. Suitable agents are well known to those skilled in the art and include, for example, parabens, chlorobutanol, phenol, benzyl alcohol, ascorbic acid, thimerosal, and the like. In many cases, it may be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminium monostearate and gelatin.

Sterile injectable solutions can be prepared by incorporating the compound in the required amount in an appropriate solvent with one or a combination of ingredients enumerated above, as required, followed by filtered sterilisation. Generally, dispersions are prepared by incorporating the comopund into a sterile vehicle which contains a basic dispersion medium and the required other ingredients from those enumerated above.

Tablets, troches, pills, capsules and the like can also contain the following: a binder such as gum gragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin or a flavouring agent such as peppermint, oil of wintergreen, or cherry flavouring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the analogue, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the analogue can be incorporated into sustained-release preparations and formulations.

Preferably, the pharmaceutical composition may further include a suitable buffer to minimise acid hydrolysis. Suitable buffer agent agents are well known to those skilled in the art and include, but are not limited to, phosphates, citrates, carbonates and mixtures thereof.

Single or multiple administrations of the pharmaceutical compositions according to the invention may be carried out. One skilled in the art would be able, by routine experimentation, to determine effective, non-toxic dosage levels of the compound and/or composition of the invention and an administration pattern which would be suitable for treating the diseases and/or infections to which the compounds and compositions are applicable.

Further, it will be apparent to one of ordinary skill in the art that the optimal course of treatment, such as the number of doses of the compound or composition of the invention given per day for a defined number of days, can be ascertained using convention course of treatment determination tests.

Generally, an effective dosage per 24 hours may be in the range of about 0.0001 mg to about 50 mg per kg body weight; suitably, about 0.001 mg to about 50 mg per kg body weight; about 0.01 mg to about 50 mg per kg body weight; about 0.1 mg to about 50 mg per kg body weight; about 0.1 mg to about 50 mg per kg body weight; or about 1.0 mg to about 50 mg per kg body weight. More suitably, an effective dosage per 24 hours may be in the range of about 1.0 mg to about 50 mg per kg body weight; about 1.0 mg to about 25 mg per kg body weight; about 5.0 mg to about 50 mg per kg body weight; about 5.0 mg to about 20 mg per kg body weight; or about 5.0 mg to about 15 mg per kg body weight.

An effective dosage per 24 hours may be in the range of about 2 mg/kg to about 30 mg/kg, about 2 mg/kg to about 5 mg/kg, about 2 mg/kg to about 10 mg/kg, about 2 mg/kg to about 15 mg/kg, about 2mg/kg to about 20 mg/kg, about 5 mg/kg to about 10 mg/kg, about 10 mg/kg to about 15 mg/kg, about 10 mg/kg to about 20 mg/kg, about 10 mg/kg to about 30 mg/kg, about 10 mg/kg to about 15 mg/kg, about 10 mg/kg to about 20 mg/kg, about 10 mg/kg to about 30 mg/kg, about 15 mg/kg to about 20 mg/kg, about 15 mg/kg to about 30 mg/kg, or about 20 mg/kg to about 30 mg/kg.

Alternatively, an effective dosage may be up to about 500 mg/m². For example, generally, an effective dosage is expected to be in the range of about 25 to about 500 mg/m², about 25 to about 350 mg/m², about 25 to about 300 mg/m², about 25 to about 250 mg/m², about 50 to about 250 mg/m², and about 75 to about 150 mg/m².

An effective dosage routine may be once a week, twice a week, thrice a week, four times a week, five times a week, six times a week or daily. An effective dosage routine may be once every 2 days, once every day or twice every day.

The compound may be administered for a duration of a day, two days, three days, four days, five days, six days, a week, two weeks, three weeks or for a month.

### EXPERIMENTAL SECTION

Non-limiting examples of the disclosure and a comparative example will be further described in greater detail by reference to specific Examples, which should not be construed as in any way limiting the scope of the invention.

### Materials and Methods

Chemicals and solvents were purchased from Sigma-Aldrich or Tokyo Chemical Industry. Ethylenediaminetetraacetic acid (EDTA) and 4-(2-hydroxyethyl)-1-piperazineethanesulfonic acid (HEPES) were purchased from Sigma-Aldrich in puriss quality. All solvents were of analytical grade and used without further purification. Clinical grade cisplatin (1 mg/mL) was purchased from Hospira Pty Ltd (Melbourne, Australia). IGEPAL CA-630, DL-dithiothreitol (DTT), tetramethylethylenediamine (TEMED), sodium deoxycholate, non-fat dried milk bovine, TWEEN® 20, ponceau S, propidium iodide (PI), Thioredoxin Reductase Assay kit (CS0170), salubrinal, formic acid and glutathione were purchased from Sigma-Aldrich (St Louis, Mo., USA). Thiazolyl Blue tetrazolium bromide (MTT) was purchased from Alfa Aesar. Tris was purchased from Vivantis Technologies. Glycine, Hyclone™ Trypsin Protease 2.5% (10×) solution, RPMI 1640, DMEM medium, Fetal bovine serum (FBS), Bovine Serum Albumin (BSA), Hank's Balanced Salt Solution (HBSS) and Pierce™ Protease and Phosphatase Inhibitor Mini Tablets were purchased from Thermo Fisher Scientific. Hyclone™ Dulbecco's Phosphate-Buffered Saline (10×) were purchased from GE Healthcare Life Sciences. Biorad protein assay dye reagent concentrate, 30% Acrylamide/Bis solution, 5× Laemmli Sample Buffer, Nitrocellulose Membrane (0.2 μm) were purchased from Bio-rad Laboratories. Luminata™ Classico and Crescendo Western HRP substrate were purchased from Merck Millipore Corporation. Milli-Q-grade purified water was obtained from a Milli-Q UV purification system (Sartorius Stedim Biotech S.A., Aubagne Cedex, France). All antibodies were obtained from Cell Signaling Technologies (Beverly, Mass., USA). Nitric acid (65% to 71%, TraceSELECT Ultra) for ICP-MS analysis were obtained from Fluka (Sigma Aldrich) and used without further purification. Au and Re standards for ICP-MS measurements were obtained from CPI international (Amsterdam, The Netherlands). Annexin V-FITC apoptosis detection reagent (500×), Z-VAD(OMe)-FMK, SP600125, U0126, cycloheximide, 3-methyladenine, SB203580, chloroquine was purchased from Abcam (Cambridge, UK). Seahorse XF^{e}24 Cell MitoStress Test Kit and Seahorse and Seahorse XF^{e}24 Glycolysis Stress Test Kit were purchased from Seahorse Bioscience. DMSO for electrochemistry was of anhydrous quality (SeccoSolv®, max. 0.025% H₂O) from Merck (Darmstadt, Germany) and used without further purification. Ferrocene (98%, Aldrich) was used as received. Tetrabutylammonium hexafluorophosphate (nBu₄NPF₆, for electrochemical analysis, >99.0%) (from Sigma-Aldrich,) were dried under reduced pressure at 70° C. for 24 hours before use. pH 7 phosphate buffer from Jenway was used as received.

¹H NMR spectra were obtained using a Bruker Avance 500 spectrometer, and the chemical shifts (δ) were reported in parts per million with reference to residual solvent peaks. Electrospray-ionization mass spectrometry (ESI-MS) spectra were obtained using a Thermo Finnigan MAT ESI-MS System. ICP-OES determination of Au content and elemental analysis were performed in Chemical, Molecular and Analysis Centre, National University of Singapore with Optima ICP-OES (Perkin Elmer, Watham, Mass., USA) and PerkinElmer PE 2400 elemental analyzer. The absorbance of thiazolyl blue tetrazolium bromide (MTT) and TrxR activity was measured by synergy H1 hybrid multimode microplate reader (Bio-Tek, Winoosky, Vt., USA). Au and Re contents in cells were determined by Agilent 7700 Series ICP-MS (Agilent Technologies, Santa Clara, Calif., USA). Flow cytometry was performed on BD LSRFortessa Cell Analyzer (BD Biosciences, Franklin Lakes, N.J., USA). Western blot images were generated from G:Box (Syngene, Cambride, UK). The UV-vis spectrophotometric measurements were obtained using a Shimadzu UV-1800 UV spectrophotometer. The UV-vis spectra in buffered aqueous solutions were recorded using a UV-3600 UV-vis-NIR spectrophotometer (Shimadzu, Japan) with a 1 cm square quartz cell at 22° C. or 37° C. (temperature was controlled using Shimadzu TCC-240A thermoelectrically temperature controller cell holder). Analytical RP-HPLC analysis was carried out with Shimadzu Prominence HPLC controlled by Dionex Chromeleon 6.60 software (Kyoto, Japan). The experimental conditions were as follows: column—Shim-Pack VP-ODS C18 column (150 mm×4.6 mm), mobile phases—acetonitrile and water, flow rate—0.3 mL/min, UV-vis detection at 233 nm. A concentration of 0.25 mM was used for the investigated complexes (injection volume 20 μL). The solvent gradient was as follows: 10-30% of acetonitrile within 0-15 min, 30-95% of acetonitrile within 15-30 min, 95-10% of acetonitrile within 35-36 min. Oxygen Consumption Rate (OCR) and Extracellular Acidification Rate (ECAR) were measured using Seahorse XF^{e}24 Cell Bioanalyzer (Seahorse Biosciences). Kinase screening was performed at the International Centre for Kinase profiling (University of Dundee, UK) according to literature procedure (Bain J, Plater L, Elliott M, Shpiro N, Hastie C J, McLauchlan H, et al. The selectivity of protein kinase inhibitors: a further update. Biochem J 2007; 408:297-315).

Single crystal X-ray diffraction studies. Single crystal X-ray diffraction was performed using either Bruker D8 Venture or Bruker SMART APEX X-ray diffractometer. Single crystals of 1met* and 2met formed after several days in mother liquor at room temperature and 4° C., respectively. Crystals were measured at low temperature (T=100 K) on a four circles goniometer using monochromatized Mo X-ray radiation (λ=0.71073 Å). Frames were integrated with the Bruker SAINT software package using a narrow-frame algorithm. Data were corrected for absorption effects using the multi-scan method implanted in the software (SADABS). Structure was solved using direct methods and subsequent differences Fourier maps, and then refined by least squares procedures on weighted F² values using the SHELXL-version 2014/6 included in WinGX system programs for Windows. All non-hydrogen atoms were assigned anisotropic displacement parameters. H-atoms were located on difference Fourier maps then introduced as fixed contributors in idealized geometry with an isotropic thermal parameters fixed at 20% higher than those carbons atoms they were attached.

NMR- and MS-based stability studies. NMR and mass spectrometric (MS) methods were employed to characterize the stability of Au complexes in DMSO and aqueous media. ¹H NMR stability studies were performed over the period of 6-10 days in d⁶-DMSO and D₂O (1met*). For MS stability studies compounds were diluted in ammonium carbonate buffer (pH 7.4, 25 mM) to a concentration of 5 μM at 37° C. The compounds were incubated with and without glutathione (1 molar eq.) and were incubated for 1, 3 and 24 hours and aliquots of these time points were subsequently further diluted 1:1 v/v with methanol to a final concentration of 2.5 μM. The samples were analysed on a Synapt G2-Si time-of-flight mass spectrometer (TOF-MS, Waters, Milford, United States) by direct infusion (DI) at a flow rate of 180 μL/h. Mass spectra were acquired and averaged over 0.5 minutes and processed using MassLynx V4.1 (Waters, Milford, United States). The instrumental parameters were as follows: 2.85 kV capillary voltage, 120° C. source temperature, 180° C. desolvation temperature, 240 L h⁻¹ desolvation gas and <6 bar nebulizer pressure.

Cyclic voltammetry and spectroelectrochemistry. Cyclic voltammetry experiments with approximately 0.5 mM solutions of investigated gold complexes in 0.1 M nBu₄NPF₆ supporting electrolyte in dimethylsulphoxide (DMSO) or in pH 7 buffered solutions were performed under argon atmosphere using a three-electrode arrangement with glassy carbon 1 mm disc working electrode (from lonode, Australia), platinum wire as counter electrode, and silver wire as pseudo-reference electrode. Ferrocene (Fc) served as the internal potential standard for DMSO and potassium hexacyanoferrate(II) for aqueous solutions. A Heka PG310USB (Lambrecht, Germany) potentiostat with a PotMaster 2.73 software package served for the potential control in voltammetry studies. In situ ultraviolet-visible-near-infrared (UV-vis-NIR) spectroelectrochemical measurements were performed on a spectrometer Avantes (Model AvaSpec-2048×14-USB2) in the spectroelectrochemical cell kit (AKSTCKIT3) with the Pt-microstructured honeycomb working electrode (1.7 mm optical path length), purchased from Pine Research Instrumentation. The cell was positioned in the CUV-UV Cuvette Holder (Ocean Optics) connected to the diode-array UV-vis-NIR spectrometer by optical fibres. UV-vis-NIR spectra were processed using the AvaSoft 7.7 software package. Halogen and deuterium lamps were used as light sources (Avantes, Model AvaLight-DH-S-BAL).

Cell lines and culture conditions. Human ovarian carcinoma cells A2780 and A2780cisR, and human breast adenocarcinoma MDA-MB-231 cells, murine hepatocytes TAMH and human cardiomyocytes AC10 were obtained from ATCC. A2780 and A2780cisR cells were cultured in RPMI 1640 medium containing 10% fetal bovine serum (FBS). MDA-MB-231, TAMH and AC10 cells were cultured in DMEM medium containing 10% FBS. A2780 and A2780cisR cells were grown in tissue culture 25 cm² flasks (BD Biosciences, Singapore) and MDA-MB-231 cells were grown in tissue culture 75 cm² flasks (BD Biosciences, Singapore) at 37° C. in a humidified atmosphere of 95% air and 5% CO₂. All stock solutions except 1met* and metformin were prepared in DMSO and the final concentration of DMSO in medium did not exceed 1% (v/v) at which cell viability was not inhibited. The amount of actual Au concentration in stock solutions was determined by ICP-OES. The stock solution of 1met* and metformin was prepared in sterile water. The stock of cisplatin was commercially available clinical grade solution in saline buffer.

Inhibition of cell viability assay. The cytotoxicity of the compounds was determined by colorimetric MTT assay. The cells were harvested from culture flasks by trypsinization and seeded into Cellstar 96-well microculture plates at the seeding density of 6000 cells per well (6×10⁴ cells/mL). TAMH and AC10 cells were seeded at 12000 and 5000 cells per well, respectively. After cells were allowed to resume exponential growth for 24 hours, they were exposed to drugs at different concentrations in media for 72 hours. The drugs were diluted in complete medium at the desired concentration and added to each well (100 μL) and serially diluted to other wells. After exposure for 72 hours, the media was replaced with MTT in media (5 mg/mL, 100 μL) and incubated for additional 45 minutes. Subsequently, the medium was aspirated and the purple formazan crystals formed in viable cells were dissolved in DMSO (100 μL). Optical densities were measured at 570 nm using the BioTek H1 Synergy microplate reader. The quantity of viable cells was expressed in terms of treated/control (T/C) values by comparison to untreated control cells, and 50% inhibitory concentrations (IC₅₀) were calculated from concentration-effect curves by interpolation. Evaluation was based on means from at least three independent experiments, each comprising six replicates per concentration level. For experiments with inhibitors, MDA-MB-231 cells were seeded at the seeding density of 8000 cells per well (8×10⁴ cells/mL). After cells were allowed to resume exponential growth for 24 hours, they were pre-treated with SP600125 (20 μM), SB203580 (20 μM), U0126 (20 μM), Z-VAD-FMK (20 μM), salubrinal (10 μM), MDIVI-1 (20 μM) or cycloheximide (12.5 μM) for 1 hours and then co-treated with 3met for 24 hours.

Cellular accumulation. Cellular accumulation of Au complexes was determined in MDA-MB-231 cells. Cells were seeded into Cellstar 6-well plates (Greiner Bio-one) at a density of 60×10⁴ cells/well (2 mL per well). After the cells were allowed to resume exponential growth for 24 hours, they were exposed to compounds of interest at various concentrations for 24 hours at 37° C. The cells were washed twice with 1 mL of PBS and lysed with RIPA lysis buffer for 5-10 min at 4° C. The cell lysates were scraped from the wells and transferred to separate 1.5 mL microtubes. The supernatant was then collected after centrifugation (13000 rpm, 4° C. for 15 minutes) and total protein content of each sample was quantified via Bradford's assay. Cell lysates were transferred to 2 mL glass vials and then digested with ultrapure 65% HNO₃ at 100° C. for 24 hours. The resulting solution was diluted to 1 mL (2-4% v/v HNO₃) with ultrapure Milli-Q water. Au content of each sample was quantified by ICP-MS. Re was used as an internal standard. Au and Re were measured at m/z 197 and m/z 186, respectively. Metal standards for calibration curve (0, 0.5, 1, 2, 5, 10, 20, 40 ppb) were freshly prepared before each measurement. All readings were made in triplicates in He mode.

Western blotting. MDA-MB-231 cells were seeded into Cellstar 6-well plates (Greiner Bio-One) at a density of 60×10⁴ cells/well (2 mL per well). After the cells were allowed to resume exponential growth for 24 hours, they were exposed to compounds of interest at various concentrations for 2, 6 and 24 hours. The cells were washed twice with PBS and lysed with RIPA buffer (50-100 μL) and kept for 5-10 minutes at 4° C. The samples were sonicated in ice bath (3 times for 30 seconds). The supernatant was then collected after centrifugation (1.3×10⁴ rpm, 4° C. for 15 minutes) and total protein content of each sample was quantified via Bradford's assay. Equal quantities of protein (50 μg) were reconstituted in loading buffer (5× Laemmli Buffer with 5% DTT) and heated at 100° C. for 10 minutes. Subsequently, the protein mixtures were resolved on a 8%, 10% or 12% SDS-PAGE gel by electrophoresis (90 V for 30 minutes followed by 120 V for 60 minutes) and transferred onto a nitrocellulose membrane (200 mA for 2 hours). The protein bands were visualised with Ponceau S stain solution and the nitrocellulose membranes were cut into strips based on the protein ladder. The membranes were washed with a TBS-T wash buffer (0.1% Tween-20 in 1×PBS) 3 times for 5 minutes. Subsequently, they were blocked in 5% BSA (w/v) in wash buffer for 1 hour and incubated with the appropriate primary antibodies in 5% BSA (w/v) in wash buffer at 4° C. overnight. The membranes were washed with a wash buffer 3 times for 7 minutes. After incubation with HRP-conjugated secondary antibodies in 2% BSA (w/v) in wash buffer (room temperature, 2 hours), the membranes were washed with a wash buffer 4× for 5 minutes. Immune complexes were detected with Luminata HRP substrates and analysed using enhanced chemiluminescence imaging. Actin was used as a loading control. The following primary monoclonal antibodies: p-mTOR, p-AMPK, BiP, CHOP, p-JNK, cleaved PARP, cleaved caspase-3, LC3, p-Ulk1(ser757), p62, β-actin and secondary HRP-linked antibody were obtained from Cell Signalling Technology. All antibodies were used at 1:2000 dilutions except secondary antibody (1:5000).

Thioredoxin reductase inhibition assay. Assay was performed according to the protocol provided by the manufacturer. Briefly, 180 μl of working buffer [100 mM pH 7.0 potassium phosphate, 10 mM EDTA, and 0.24 mM NADPH], 8 μl of 1× assay buffer [100 mM pH 7.0 potassium phosphate and 10 mM EDTA], and 2 μl of total rat TxR enzyme solution (10 ng) were added to each well of a Corning clear 96-well plate. Compounds of interest were serially diluted at the separate 96-well plate and 4 μl of each concentration was added to the appropriate wells. The reaction mixture was incubated with gentle shaking (room temperature, 30 minutes). Subsequently, 6 μl of DNTB (100 mM) was added and incubated for an additional 3 minutes. Thereafter, the absorbance at 412 nm was measured every minute for 30 minutes. % enzyme activity at each concentration was calculated from the data obtained with reference to the control sample and half-maximal effective concentration (EC₅₀) was calculated from concentration-effect curves by interpolation.

Annexin V/PI apoptosis assay. MDA-MB-231 cells were seeded into Cellstar 12-well plates (Greiner Bio-One) at a density of 20×10⁴ cells/well (1 mL per well). The cells were allowed to resume exponential growth for 24 hours and subsequently they were exposed for 24 hours to compounds of interest at concentrations corresponding to 1.5 IC₅₀ values. After the supernatant solution was collected in 1.5 mL microtubes, the cells were washed with 100 μl of trypsin, which was combined with the supernatant. Subsequently, cells were trypsinized with 200 μl trypsin for 5 minutes at 37° C., 5% CO₂, washed with 200 μl of PBS and combined with the supernatant. The cells were centrifuged at 2.5×10³ rpm for 5 minutes and the pellets were washed once with PBS and resuspended in 500 μl of Annexin V binding buffer and stained with Annexin V-FITC and PI reagents. The fluorescence was immediately analyzed by flow cytometry. The resulting dot blots were acquired from 10000 events and quantified using Flowjo software (Flowjo LLC, Ashland, Oreg., USA).

Mitochondrial bioenergetics (MitoStress assay): Oxygen Consumption Rate (OCR) was monitored by using Seahorse XF^{e}24 Cell MitoStress Test Kit (Seahorse Bioscience). Prior to the assay, XF sensor cartridges were hydrated. MDA-MB-231 cells were seeded onto XF^{e}24-well cell culture plates at a density of 4×10⁴ cells/well in 250 μL DMEM complete growth medium and then incubated for 48 h at 37° C. in 5% CO₂ incubator. The cells were subsequently exposed to compounds of interest at different concentrations for 24 hours. The wells were washed with 250 μL of pre-warmed Seahorse XF DMEM media, supplemented with 10 mM glucose, 1 mM sodium pyruvate and 2 mM glutamine, pH 7.4 and replaced with 525 μL of this media. The plate was incubated at 37° C. in a non-CO₂ incubator for 1 hour before the measurement. Three baseline measurements of OCR were recorded and 1 μM of olygomycin (ATP synthase complex inhibitor), 1 μM of FCCP (ATP synthesis uncoupler trifluorocarbonylcyanide phenylhydrazone) and a mixture of 0.5 μM of antimycin-A (inhibitor of complex III) and 0.5 μM of rotenone (inhibitor of electron transfer from Fe-S center of complex I to ubiquinone) were added in sequence. OCR was measured using Seahorse XF^{e}24 Cell Bioanalyzer (Seahorse Biosciences). The media was removed from the wells and 60 μL of 0.1 M NaOH was added. After vigorous pipetting, the protein content was quantified by BCA assay and OCR was normalized per protein content.

Glycolysis Stress Test assay: Extracellular acidification rate (ECAR) was monitored by using Seahorse XF^{e}24 Glycolysis Stress Test Kit (Seahorse Bioscience). Prior to the assay, XF sensor cartridges were hydrated. MDA-MB-231 cells were seeded onto XF^{e}24-well cell culture plates at a density of 4×10⁴ cells/well in 250 μL DMEM complete growth medium and then incubated for 48 hours at 37° C. in 5% CO₂ incubator. The cells were subsequently exposed to compounds of interest at different concentrations for 24 hours. The wells were washed with 250 μL of pre-warmed Seahorse XF DMEM media, supplemented with 100 mM glutamine, pH 7.4 and replaced with 525 μL of this media. The plate was incubated at 37° C. in a non-CO₂ incubator for 1 hour before the measurement. Three baseline measurements of ECAR were recorded and 10 mM of glucose, 1 μM of olygomycin and 100 mM of 2-deoxyglucose (2-DG) were added in sequence. ECAR was measured using Seahorse XF^{e}24 Cell Bioanalyzer (Seahorse Biosciences). The media was removed from the wells and 60 μL of 0.1 M NaOH was added. After vigorous pipetting, the protein content was quantified by BCA assay and OCR was normalized per protein content.

Animal experiments: 6-week-old female athymic nude mice were purchased from Invigo. Animals were housed in animal-holding units at Center for Comparative Medicine, Northwestern University in a pathogen-free environment at constant temperature in a 12/12-hour light/dark cycle. All animal procedures were carried out in accordance with a protocol approved by the Institutional Animal Care and Use Committee, Protocol number: IS00002999. Animals were acclimatized for 2 weeks following arrival before the beginning of experiments. Mice were allowed free access to food and water. To identify the MTD, mice were daily injected via i.p. route with 5, 10, 15 and 20 mg/kg of 3met (3-4 mice per group) for 4-5 days. All mice were bright, alert and responsive during the whole study. On day 6, only mice receiving 20 mg/kg demonstrated weight loss (11%) with no other signs of toxicity; therefore, the MTD chosen for activity study was 15 mg/kg. MDA-MB-231 cells expressing firefly luciferase were collected by trypsinization and mixed with Matrigel (1:1) for fat pad injections. The MDA-MB-231 cell/matrigel mixture (100 μI) was injected into the fat pad of the fourth mammary glands on both sides of 6-week-old female athymic nude-Foxn1^{nu} mice (Envigo) at 2.5×10⁵ cells/gland under ketamine/xylazine anesthesia (50-100/5-10 mg/kg). Ketaprofen and Buprenophrine SR were injected subcutaneously for pain management at 2-5 mg/kg and 0.5-1 mg/kg, respectively. The growth of tumors was monitored by live animal imaging. Once tumors became palpable (week 2 after tumor implantation), treatment was initiated. Animals were separated into two groups (7 mice in each group) and tumor dimensions were measured using a caliper by width×length (mm²). Drug-treated group was injected with 15 mg/kg of 3met in 0.9% sterile saline with 10% DMSO, whereas control group was injected with 0.9% sterile saline with 10% DMSO as a vehicle. Treatment was performed on days 14, 16 and 18 and repeated in a similar fashion during weeks 4 and 5. Animals were sacrificed on week 6. Animals were controlled for distress development. Their weight changes were monitored every 2-3 days for 6 weeks. The transient weight loss was observed in a drug-treated group on day 18, which returned back to normal at week 4. Two mice died in a drug-treated group and one mouse was sacrificed during anaesthesia. Statistical analysis was done by Student's unpaired T-test and One-way Anova test with post-hoc Dunnett test using GraphPad Prism 9 software.

Organ distribution studies: At the experimental endpoint, the hearts were perfused with PBS immediately before the excision of the organs. Brain, liver, kidney, spleen, heart, lung, bone and tumor tissues were collected from each mouse and flash-frozen in liquid nitrogen. The organs of the vehicle-treated mouse were used for the measurements of the background. The organs were lyophilized for 4 days, weighed, mechanically minced and digested in 100 μl of ultrapure 65% HNO₃ at 105° C. for 3 days. The resulting solution was diluted to 2% v/v HNO₃ with ultrapure Milli-Q water. Gold content was measured by ICP-MS as described above.

Histopathological examination: Tumor, liver and kidney tissues were fixed in 10% buffered formalin overnight and washed twice with PBS. Tissues were then embedded in paraffin. Sections with a thickness of 4 μm were prepared, deparaffinised in xylene (twice) and rehydrated in a graded series of alcohol (two times 100% alcohol and 75% alcohol) and distilled water. Later, sections were stained with a haematoxylin solution, rinsed in water, passed through a 70% ethanol solution containing 1% HCl, and rinsed again with tap water. Sections were stained with eosin for 5 minutes and rinsed with absolute alcohol and xylene for 5 minutes. The slides were scanned using Hamamatsu K.K. Nanozoomer 2.0 HT instrument. All tissue slides were processed with QuPath software. For tumor slides algorithms for tumor and necrosis quantification with random trees pixel classifiers were used with color designation as red is tumor, green is stroma and necrosis is black.

### Synthesis of Cyclometalated Au^{III} Complexes

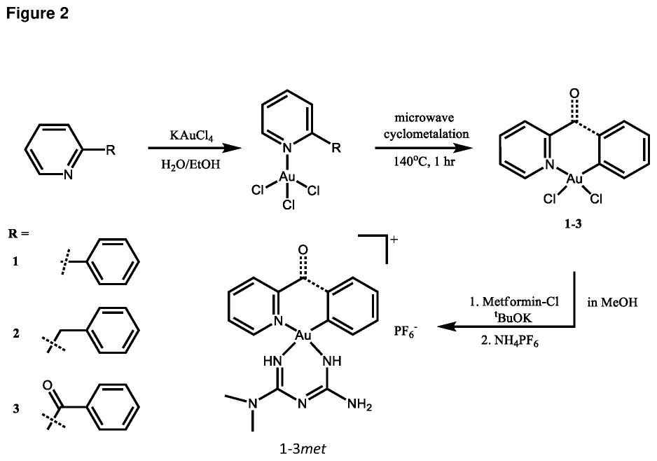

Cyclometalated Aukdichlorido precursors with the general formula [Au^{III}(C\hat{ }N)Cl₂] 1-3 (C\hat{ }N=2-phenylpyridine (1), 2-benzylpyridine (2) and 2-benzoylpyridine (3)) were prepared from KAuCl₄ under microwave conditions or under reflux with AgOTf and subsequently reacted with 2 eq. of metformin or phenformin hydrochloride in methanol (FIG. 2). For precursor 3, the reflux synthesis yielded significantly better results compared to the microwave synthesis.

Microwave synthesis: To a clear yellow solution of KAuCl₄ (300 mg, 0.80 mmol) in ultrapure water (5 ml), 1.05 eq. of either 2-phenylpyridine (1) (120 μl, 0.84 mmol), 2-benzylpyridine (2) (134 μl, 0.84 mmol) or 2-benzoylpyridine (3) (153 mg, 0.84 mmol) in 1 ml of ethanol was added dropwise which yielded a yellow precipitate. The reaction mixture was sonicated for 15 minutes before being subjected to microwave irradiation at a variable power with constant temperature for 45 minutes with vigorous stirring (140° C., 70-90 bar). No changes in colour were observed. The suspension was centrifuged and yellow supernatant was discarded. The solid residue was washed with copious amounts of water (15 ml×3), until it became white. Then it was washed with methanol (15 mL×2) and diethyl ether (5 mL×3) before being dried in vacuo. The crude products were used for further synthesis without additional purification. The purity of the products was confirmed by analytical HPLC.

Reflux synthesis: To a clear yellow solution of KAuCl₄ (300 mg, 0.80 mmol) in ultrapure water (5 ml), 1.05 eq. of either 2-phenylpyridine (1) (120 μl, 0.84 mmol), 2-benzylpyridine (2) (134 μl, 0.84 mmol) or 2-benzoylpyridine (3) (153 mg, 0.84 mmol) in 1 ml of ethanol was added dropwise which yielded a yellow precipitate. A yellow precipitate was isolated, washed with diethyl ether and dried. 1 eq. of each intermediate was dissolved in 7 ml of acetonitrile and 1.05 eq. of silver triflate was added. The reaction mixture was refluxed at 85° C. overnight. The hot filtrate was filtered to remove impurities and concentrated in vacuo to provide a precipitate, which was filtered off and washed with acetonitrile (5 ml), methanol (5 ml) and diethyl ether (5 mL×3) before being dried in vacuo.

(2-phenylpyridine)AuCl₂ (scaffold 1): Yield: 230 mg (65%). ¹H NMR (400 MHz, DMSO-d⁶): δ (ppm)=9.84 (dd, J=4.8, 1.2 Hz, 1H), 8.14 (td, J=5.9, 1.2 Hz, 1H), 8.07 (dd, J=6.0, 0.9 Hz, 1H), 7.91 (dd, J=5.1, 0.9 Hz, 1H), 7.56 (dd, J=5.7, 1.2 Hz, 1H), 7.51 (td, J=5.3, 0.9 Hz, 1H), 7.41 (dd, J=5.9, 0.9 Hz, 1H), 7.38 (m, 1H).

(2-benzylpyridine)Au^{III}Cl₂ (2): Yield: 30%. RP-HPLC (% Purity): >99.9%, tᵣ=7.8 min. ¹H NMR (300 MHz, DMSO-d⁶): δ (ppm)=9.17 (dd, ³J_{H-H}=6.0 Hz, ⁴J_{H-H}=1.5 Hz, 1H), 8.26 (td, ³J_{H-H}=7.8 Hz, ⁴J_{H-H}=1.5 Hz, 1H), 7.99 (dd, ³J_{H-H}=7.8, ⁴J_{H-H}=1.5 Hz, 1H), 7.69 (ddd, ³J_{H-H}=7.8 Hz, ³J_{H-H}=5.7 Hz, ⁴J_{H-H}=1.5 Hz, 1H), 7.41 (dd, ³J_{H-H}=8.0 Hz, ⁴J_{H-H}=1.6 Hz, 1H), 7.25 (dd, ³J_{H-H}=7.5 Hz, ⁴J_{H-H}=1.8 Hz, 1H), 7.18 (td, ³J_{H-H}=7.5 Hz, ⁴J_{H-H}=1.8 Hz, 1H), 7.08 (ddd, ³J_{H-H}=7.8 Hz, ³J_{H-H}=7.5 Hz, ⁴J_{H-H}=1.8 Hz, 1H), 4.60 (d, ²J_{H-H}=15.3 Hz, 1H), 4.33 (d, ²J_{H-H}=15.3 Hz, 1H).

(2-benzoylpyridine)Au^{III}Cl₂ (3): Yield: 27%. RP-HPLC (% Purity): >99.9%, tᵣ=7.9 min. ¹H NMR (300 MHz, DMSO-d⁶): δ (ppm)=9.48 (dd, ³J_{H-H}=6.0 Hz, ⁴J_{H-H}=1.2 Hz, 1H), 8.55 (td, ³J_{H-H}=7.8 Hz, ⁴J_{H-H}=1.2 Hz, 1H), 8.36 (dd, ³J_{H-H}=7.8, ⁴J_{H-H}=1.5 Hz, 1H), 8.08 (ddd, ³J_{H-H}=7.8 Hz, ³J_{H-H}=5.7 Hz, ⁴J_{H-H}=1.2 Hz, 1H), 7.76 (m, 1H), 7.69 (m, 1H), 7.49 (m, 2H).

The [Au^{III}(C\hat{ }N)Cl₂] 1-3 complexes were subsequently reacted with 2 eq. of metformin or phenformin hydrochloride and 4 eq. of ^{t}BuOK in methanol, yielding five novel organometallic Au^{III}-metformin and phenformin complexes (1-3met, 1phen and 1met*, FIG. 1 and FIG. 2). The specific synthetic steps and characterization are as follows:

[Au^{III}(2-phenylpyridine)(metformin)]Cl, (1met*): To 1 (0.2 mmol, 84 mg) was added 2 eq. of metformin hydrochloride (0.4 mmol, 67 mg) and 4.25 eq. of potassium tert-butylbutoxide (0.85 mg, 95 mg) in methanol (10 ml) and the mixture was stirred overnight at room temperature under protection from light. Quick precipitation with gradual change of colour from yellow to white was observed. The suspension was centrifuged and the white solid residue was washed with water (2 ml×1), methanol (5 ml×1) and diethyl ether (5 ml×3) and dried in vacuo to give 54 mg of the final product. Yield: 52% (C₁₅H₁₈AuN₆Cl—514.09 g mol⁻¹). ¹H NMR (300 MHz, D₂O, major isomer): δ (ppm)=7.97 (m, 1H), 7.55 (m, 2H), 7.30 (m, 1H), 7.21 (m, 1H), 7.07 (m, 1H), 6.92 (m, 1H), 6.48 (d, ³J_{H-H}=7.8 Hz, 1H), 2.81 (s, 6H, —NMe₂). Ratio of geometric isomers: 3:1. ESI-MS (+ve, m/z): calculated for: 479.1 [M⁺=C₁₅H₁₈AuN₆⁺], found 479.0. ESI-MS/MS (+ve, m/z): calculated 434.1 [M—H—N(CH₃)₂]⁺, found 434.0. HR-ESI-MS for [M⁺=C₁₅H₁₈AuN₆⁺]: calculated 479.1258, found 479.1258. Anal. Calculated for C₁₅H₁₈AuClN₆*CH₃OH*H₂O: C 34.03, H 4.29, N 14.89. Found: C 33.90, H 4.62, N 14.83. It should be noted that 1met* can be used interchangeably with 1Met* and 1met-Cl.

[Au^{III}(2-phenylpyridine)(metformin)]PF₆, (1met): To a suspension of 1met* (0.10 mmol, 50 mg) in H₂O (6 ml) was added solution of NH₄PF₆ (0.40 mmol, 65 mg) in H₂O (0.5 ml) and stirred at room temperature for 1 hour under protection from light. A white jelly precipitate was formed. The solution was filtered to give a purple-white solid which was washed with water (2 ml×2), dichloromethane (3 mL×3) and diethyl ether (10 mL×3) and dried in vacuo. Yield: 89% (C₁₅H₁₈AuN₆PF₆—624.09 g mol⁻¹. The purity of the product was confirmed by analytical HPLC (retention time 8.0 min) and HR-ESI-MS. ¹H NMR (300 MHz, DMSO-d⁶): δ=8.85 (d, ³J_{H-H}=6.2 Hz, 1H, ppy), 8.42 (m, 2H), 8.09 (m, 1H), 7.70 (m, 2H), 7.51 (m, 2H), 6.60 (s, 2H, NH₂), 6.07 (s, 1H, NH), 5.93 (s, 1H, NH), 3.12 (s, 6H, NMe₂). ESI-MS (+ve, m/z): calculated 479.1 [M⁺=C₁₅H₁₈AuN₆⁺], found 479.0. ESI-MS (−ve, m/z): calculated 145.0 [PF₆⁻], found 145.1. ESI-MS/MS (+ve, m/z): calculated 434.1 [M—H—N(CH₃)₂]⁺, found 434.0. HR-ESI-MS for [M⁺=C₁₅H₁₈AuN₆⁺]: calculated 479.1258, found 479.1255. It should be noted that 1met can be used interchangeably with 1Met and 1met-PF₆.

2met, 3met and 1phen: To 1 (0.15 mmol, 63 mg), 2 (0.15 mmol, 65 mg) or 3 (0.15 mmol, 67 mg) was added 2 eq. of phenformin hydrochloride (0.30 mmol, 72 mg) or metformin hydrochloride (0.30 mmol, 49 mg) and 4 eq. of potassium tert-butylbutoxide (0.60 mmol, 67 mg) in methanol (10 ml), and the mixture was stirred overnight at r. t. under protection from light. No changes in colour were observed. The solution was centrifuged to remove any insoluble residues, placed on ice and solution of NH₄PF₆ (0.60 mmol, 98 mg) in methanol (0.5 ml) was added dropwise. Subsequently, Et₂O (30 ml) was added and solution was left at 4° C. for 24 to 72 hours to give white or yellow precipitate, which was filtered and washed with water or methanol (2 ml×2), dichloromethane (3 mL×2) and diethyl ether (10 mL×3) and dried in vacuo. Crystals suitable for X-ray diffraction analysis were grown by slow diffusion of diethyl ether into a methanol solution. The purity of the products was confirmed by analytical HPLC and HR-ESI-MS.

Au^{III}(2-phenylpyridine)(phenformin)]PF₆, (1phen): Yield: 49% (C₂₁H₂₂AuN₆PF₆—700.12 g mol⁻¹). RP-HPLC: tᵣ=8.0 min. ¹H NMR (500 MHz, DMSO-d⁶, major isomer): δ=8.81 (m, 1H), 8.43 (m, 2H), 8.07 (m, 1H), 7.79 (m, 1H, ppy), 7.68 (m, 1H), 7.50 (m, 2H), 7.37−7.26 (m, 5H), 7.10 (s, 1H, NH), 6.93 (s, 1H, NH), 6.68 (s, 2H, NH₂), 3.53 (m, 2H, CH₂), 2.82 (m, 2H, CH₂). Ratio of geometric isomers: 3:1. ESI-MS (+ve, m/z): calculated 555.2 [M⁺=C₂₁H₂₂AuN₆⁺], found 555.1. ESI-MS (−ve, m/z): calculated 145.0 [PF₆⁻], found 145.1. ESI-MS/MS (+ve, m/z): calculated 434.1 [M—H—NH—C₂H₄—C₆H₅]⁺, found 434.0. HR-ESI-MS for [M⁺=C₂₁H₂₂AuN₆⁺]: calculated 555.1571, found 555.1559. It should be noted that 1phen can be used interchangeably with 1Phen and 1phen-PF₆

Au^{III}(2-benzylpyridine)(metformin)]PF₆, (2met): Yield: 42% (C₁₆H₂₀AuN₆PF₆—638.11 g mol⁻) RP-HPLC: tᵣ=7.9 min. ¹H NMR (300 MHz, DMSO-d⁶, major isomer): δ=8.91 (d, ³J_{H-H}=5.5 Hz, 1H), 8.26 (t, ³J_{H-H}=7.5 Hz, 1H), 8.00 (d, ³J_{H-H}=8.0 Hz, 1H), 7.68 (t, ³J_{H-H}=8.0 Hz, 1H), 7.33 (d, ³J_{H-H}=7.0 Hz, 1H), 7.30 (d, ³J_{H-H}=7.0 Hz, 1H), 7.21 (m, 2H), 6.38 (s, 2H, NH₂), 6.19 (s, 1H, NH), 6.07 (s, 1H, NH), 4.51 (d, ²J_{H-H}=15.0 Hz, 1H, CH₂), 4.33 (d, ²J_{H-H}=15.0 Hz, 1H, CH₂), 3.02 (s, 6H, NMe₂). Ratio of geometric isomers: 11:1. ESI-MS (+ve, m/z): calculated 493.1 [M+=C₁₆H₂₀AuN₆⁺], found 493.1. ESI-MS (−ve, m/z): calculated 145.0 [PF₆⁻], found 145.1. ESI-MS/MS (+ve, m/z): calculated 448.1 [M—H—N(CH₃)₂]⁺, found 448.0. HR-ESI-MS for [M⁺=C₁₆H₂₀AuN₆⁺]: calculated 493.1415, found 493.1403. It should be noted that 2met can be used interchangeably with 2Met and 2met-PF₆.

Au^{III(}2-benzoylpyridine)(metformin)]PF₆, (3met): Yield: 48% (C₁₆H₁₈AuN₆OPF₆—652.08 g mol⁻¹). RP-HPLC: tᵣ=8.1 min. ¹H NMR (300 MHz, DMSO-d⁶, major isomer): δ=8.92 (d, ³J_{H-H}=5.5 Hz, 1H), 8.18 (m, 2H), 7.83 (m, 3H), 7.61 (m, 1H), 7.48 (t, ³J_{H-H}=7.5 Hz, 1H), 7.11 (s, 1H, NH), 6.59 (s, 1H, NH), 6.49 (s, 2H, NH₂), 3.06 (s, 6H, NMe₂). Ratio of geometric isomers: 2:1. ESI-MS (+ve, m/z): calculated 507.1 [M⁺=C₁₆H₁₈AuN₆O⁺], found 506.0. ESI-MS (−ve, m/z): calculated 145.0 [PF₆⁻], found 145.1. ESI-MS/MS (+ve, m/z): calculated 462.1 [M—H—N(CH₃)₂]⁺, found 461.0. It should be noted that 3met can be used interchangeably with 3Met and 3met-PF₆.

Complexes 1-3met and 1phen were isolated as PF₆⁻ salts in moderate yields. Additionally, the water-soluble Au^{III}-metformin complex with 2-phenylpyridine 1met* was isolated as a Cl⁻ salt by taking advantage of its relatively poor solubility in methanol and other organic solvents, resulting in direct precipitation from the reaction media. The solubility in methanol of all PF₆⁻ complexes was completely different from that of the Cl⁻ complex. Only the Cl⁻ complex was precipitated by methanol. All other compounds had to be precipitated by diethyl ether.

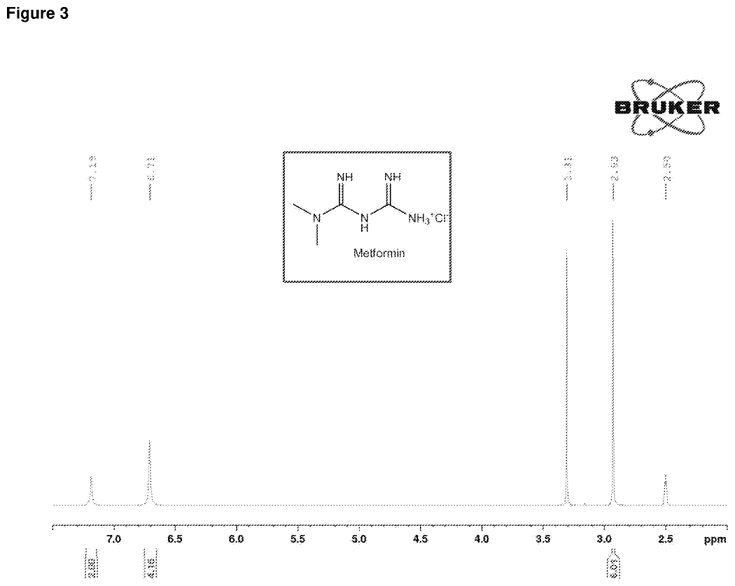

Upon coordination of the asymmetric metformin or phenformin to an Au^{III} centre, complexes formed racemic mixtures of E- and Z- isomers, as was evidenced by two independent sets of ¹H NMR signals. Detailed synthesis and characterization of Au^{III} complexes are presented in FIGS. 3 to 24, as summarized below in Table 1:

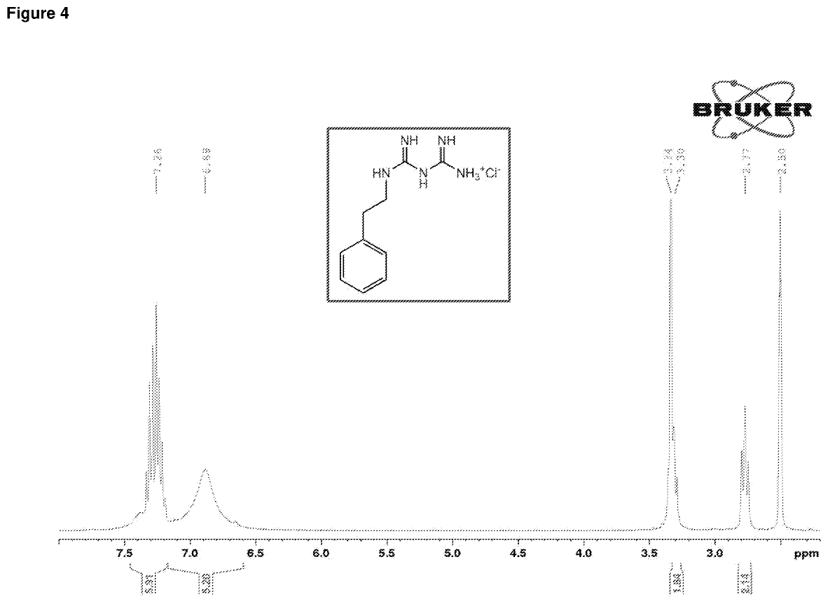

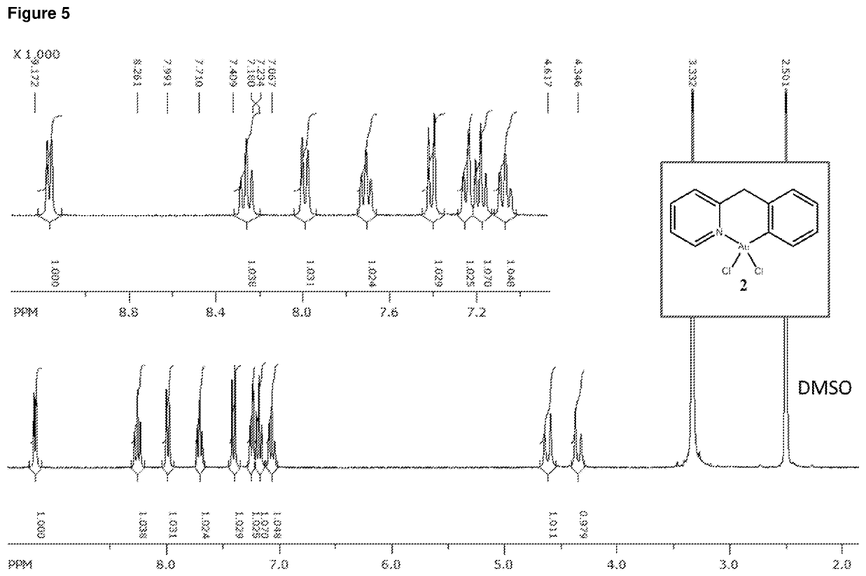

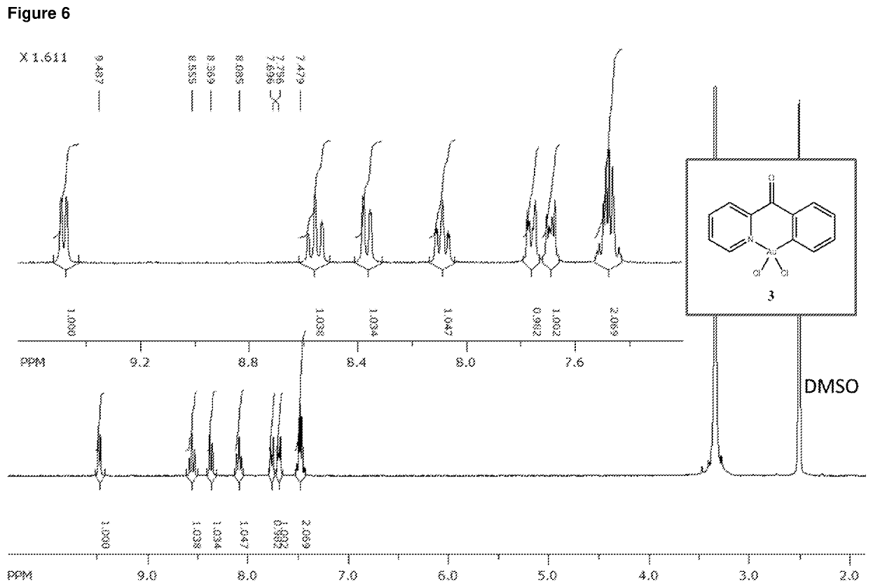

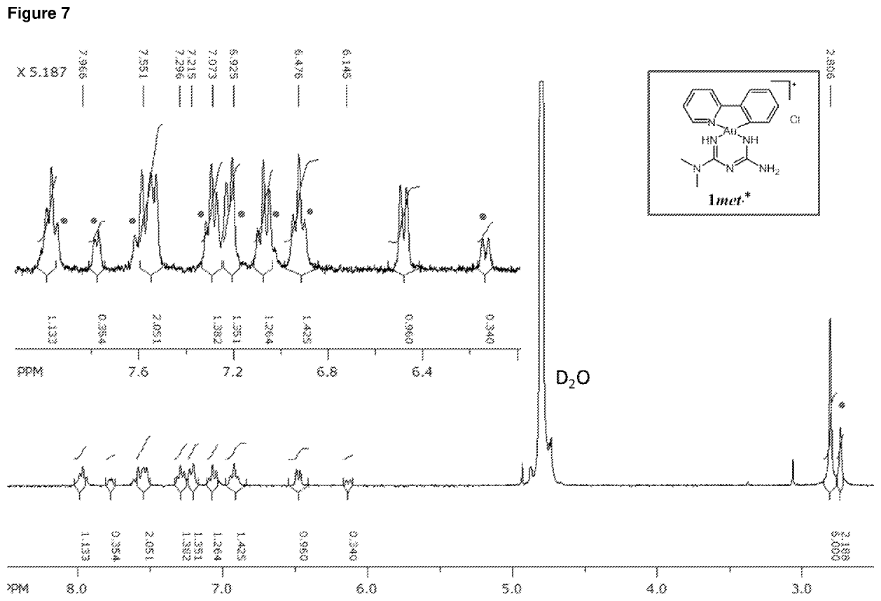

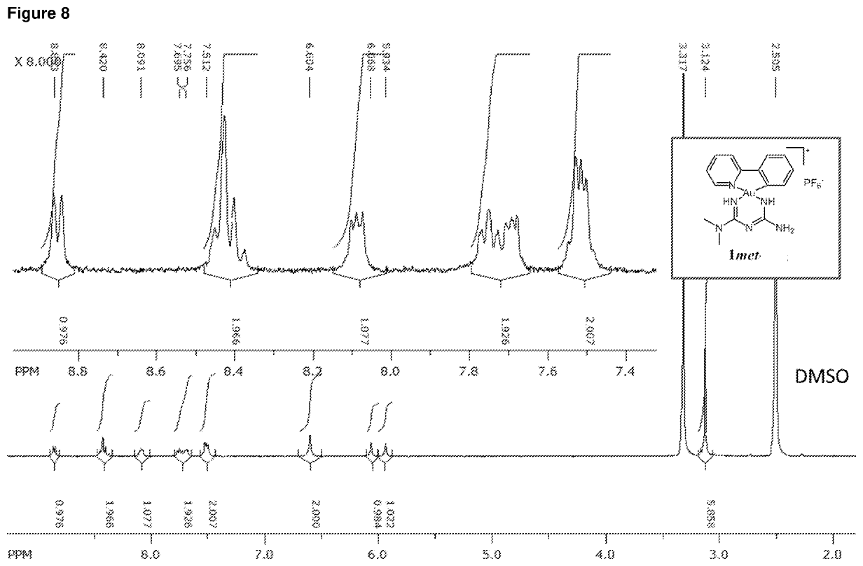

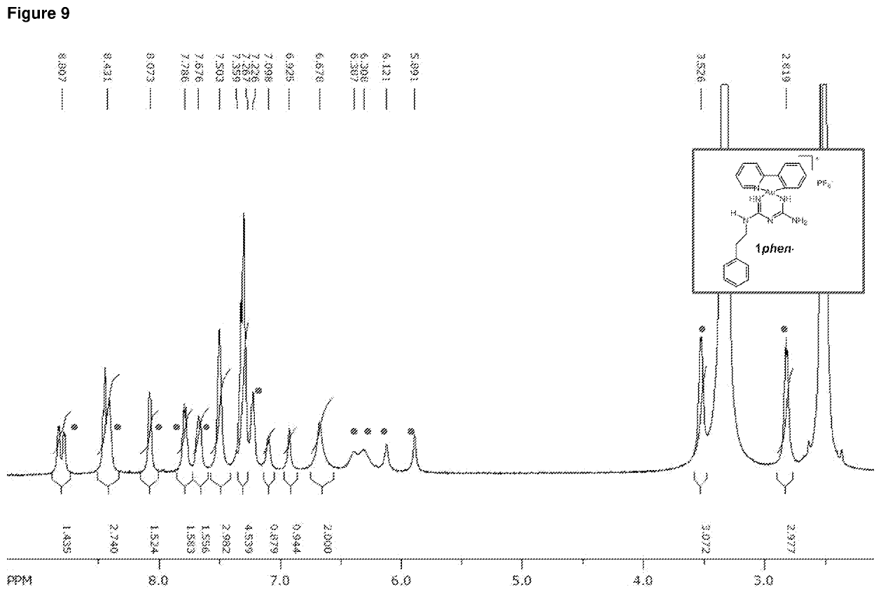

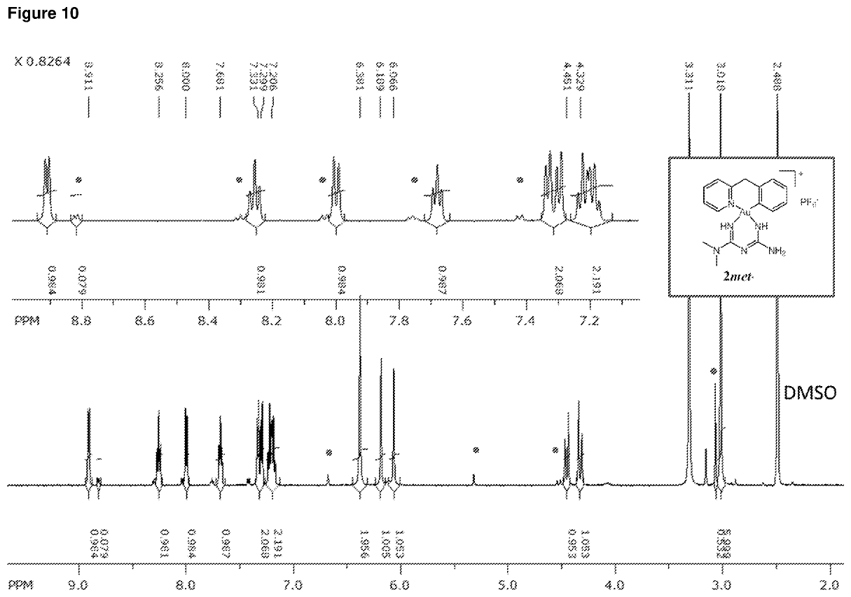

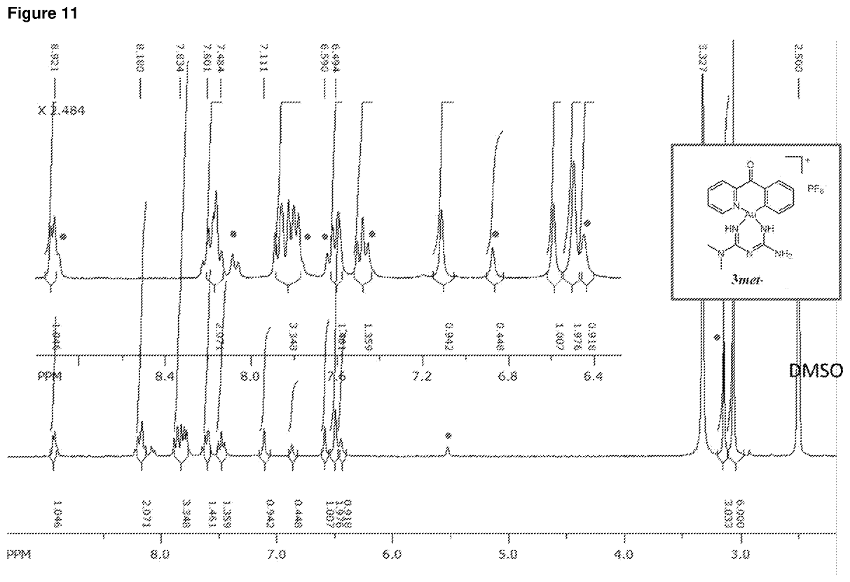

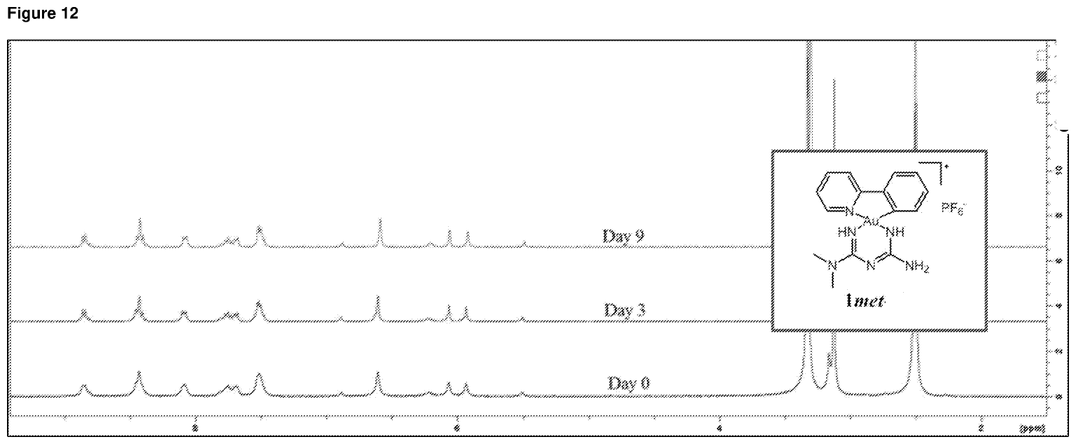

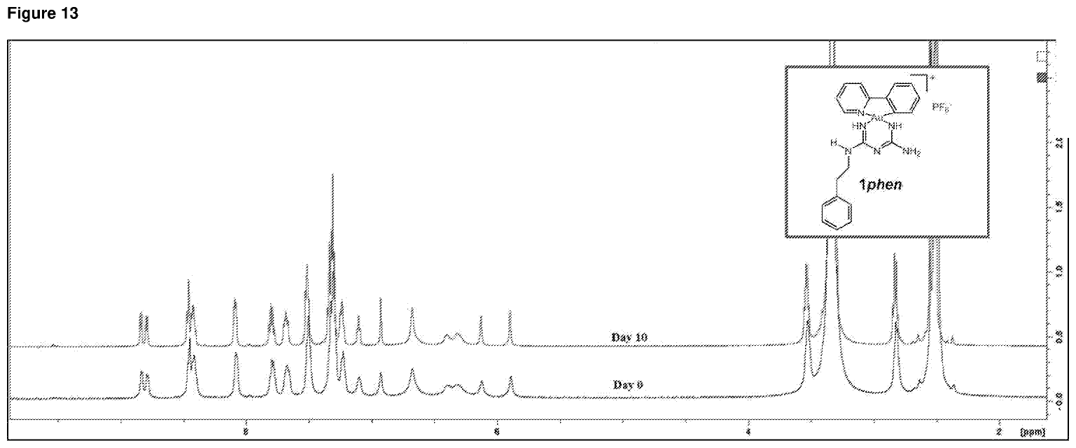

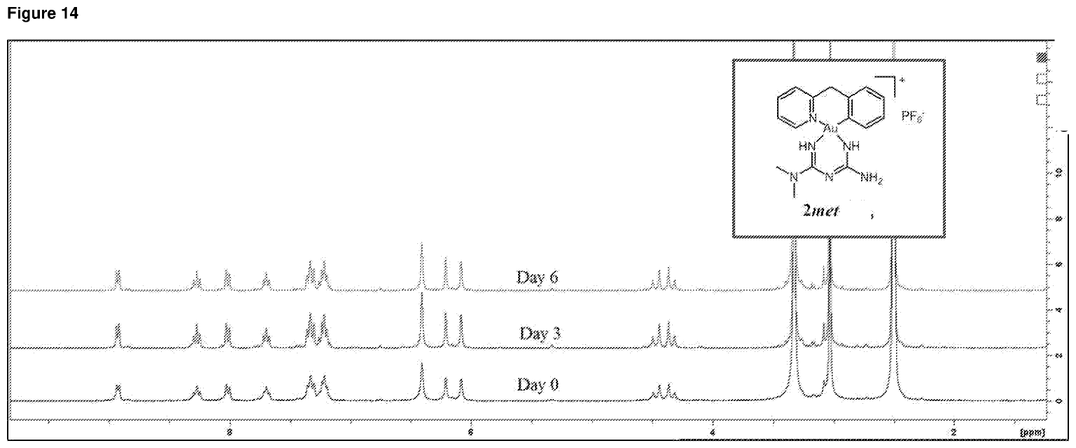

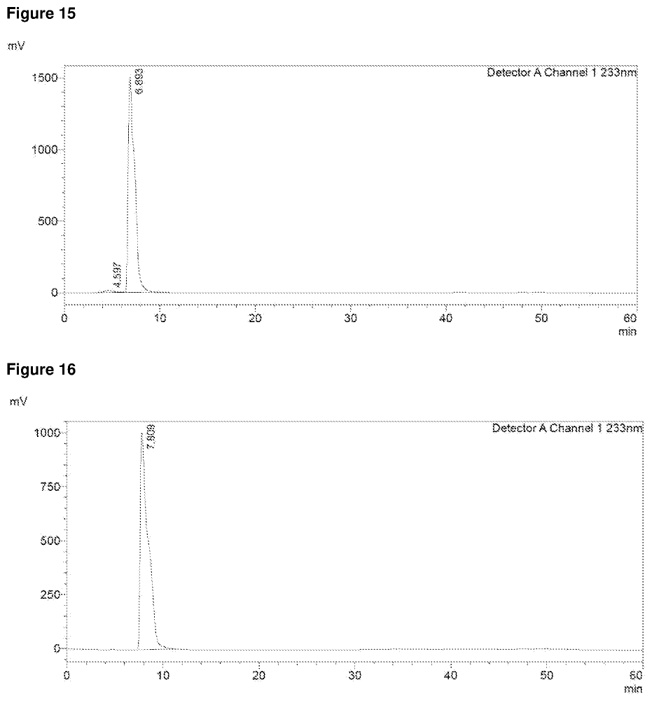

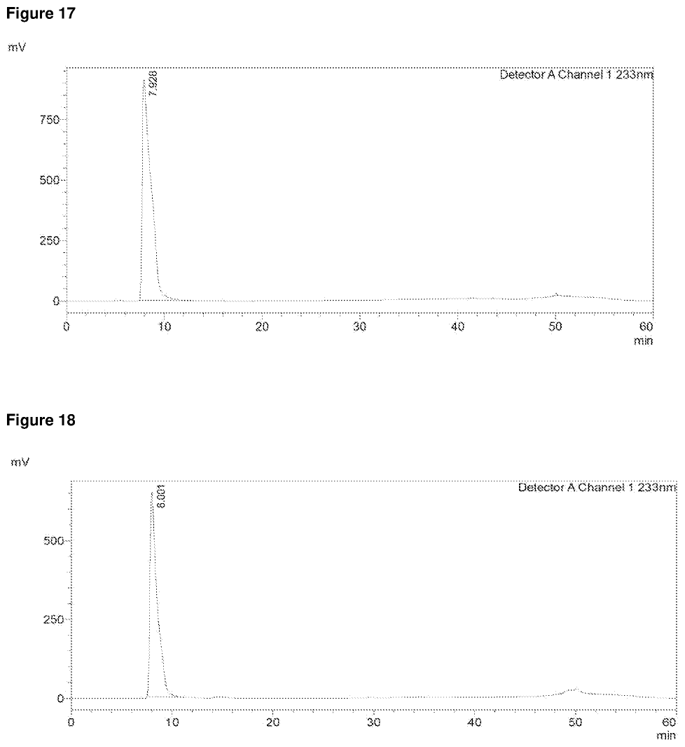

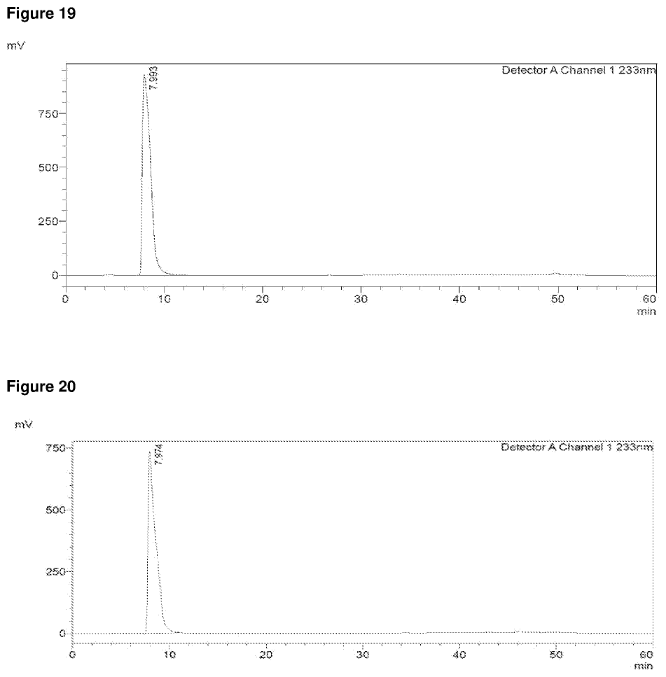

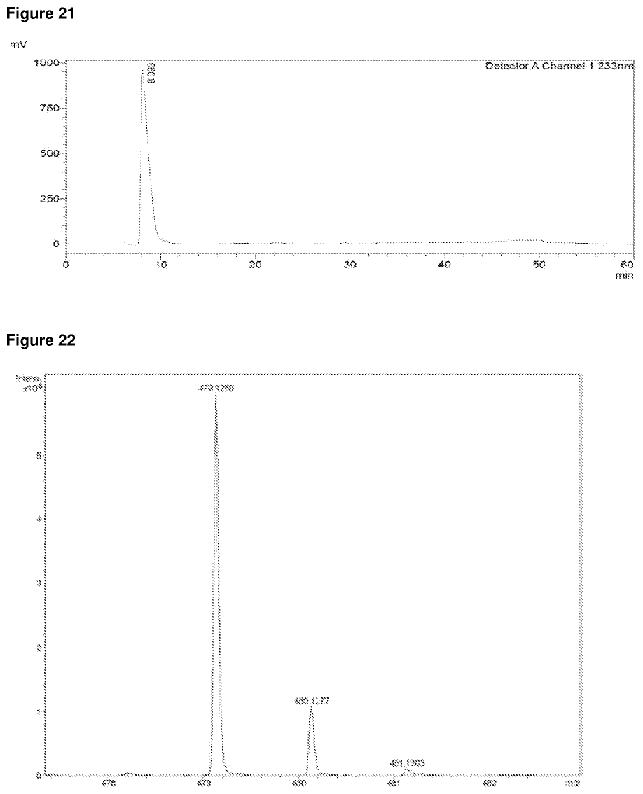

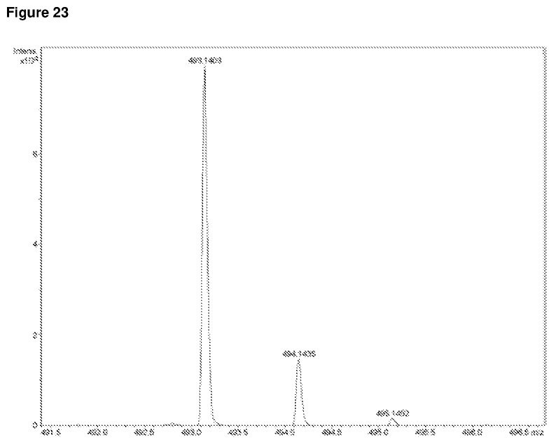

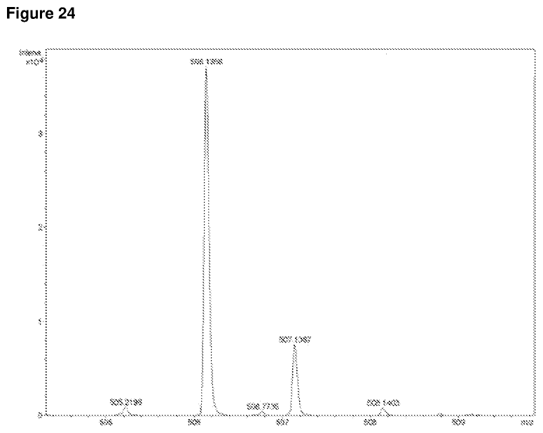

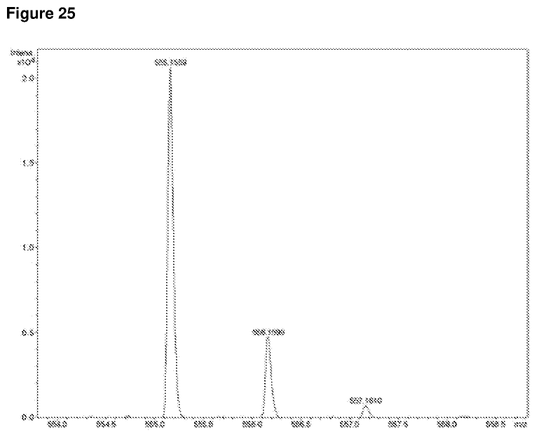

TABLE 1
Summary of the characterization of the
compounds used and complexes made

| | | 1H NMR | | |
|---|---|---|---|---|
| | 1H NMR | stability | | High resolution |
| Compound | spectrum | study | RR-HPLC | ESI-MS |
| Metformin | FIG. 3 | — | — | — |
| Phenformin | FIG. 4 | — | — | — |
| 1 | — | — | FIG. 15 | — |
| 2 | FIG. 5 | — | FIG. 16 | — |
| 3 | FIG. 6 | — | FIG. 17 | — |
| 1met* | FIG. 7 | — | | — |
| 1met | FIG. 8 | FIG. 12 | FIG. 18 | FIG. 22 |
| 1phen | FIG. 9 | FIG. 13 | FIG. 19 | FIG. 25 |
| 2met | FIG. 10 | FIG. 14 | FIG. 20 | FIG. 23 |
| 3met | FIG. 11 | — | FIG. 21 | FIG. 24 |

Purity was assessed by RP-HPLC or elemental analysis and shown to be >98% pure for all complexes (FIGS. 15 to 21). The solid-state structures of 1met* and 2met were analyzed by X-ray diffraction analysis (Tables 2 and 3) which confirmed the classical square planar coordination geometry of the Au^{III} centre.

TABLE 2
Key bond lengths and angles observed in the molecular structures of 1met* (left)
and 2met (right) as compared to uncoordinated metformin, 1, dimethylated analogue of 2
([Au{NC₅H₄(CMe₂C₆H₄)}Cl₂]) and 3, taken from Cambridge Structural Database.

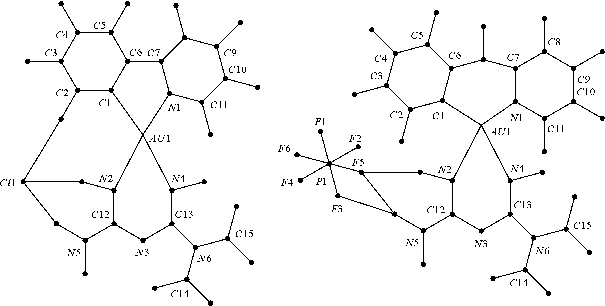

| Bond lengths | Compound | | | | | |
|---|---|---|---|---|---|---|
| (Å) and angles | Metformin | | | | | |
| (°) | •HCl^{(a)} | 1^{(b)} | 1met* | 2-Me₂^{(c)} | 2met | 3^{(d)} |
| CCDC number | 1182489 | 206807 | 1939306 | 1311518 | 1939307 | 1239251 |
| Au1-C1/Å | | 1.950(2) | 2.022(3) | 2.021(3) | 2.028(2) | 2.033(8) |
| Au1-N1/Å | | 2.034(13) | 2.038(2) | 2.040(3) | 2.036(2) | 2.035(5) |
| Au1-N2/Å | | | 1.972(2) | | 1.974(2) | |
| Au1-N4/Å | | | 2.039(2) | | 2.034(2) | |
| Au1-Cl1/Å | | 2.361(8) | | 2.390(1) | | 2.381(2) |
| Au1-Cl2/Å | | 2.282(5) | | 2.282(1) | | 2.275(2) |
| C-C (aromatic, | | 1.38 | 1.3882 | 1.383 | 1.3869 | 1.3812 |
| average.)/Å | | | | | | |
| C5-C6/Å | | 1.42(3) | 1.402(4) | 1.398(7) | 1.394(4) | 1.39(1) |
| C7-C16/Å | | | | 1.532(5) | 1.498(4) | 1.501(10) |
| C6-C16/Å | | | | 1.541(4) | 1.516(4) | 1.486(10) |
| N1-C11/Å | | 1.32(2) | 1.344(4) | 1.348(6) | 1.359(3) | 1.34(1) |
| N1-C7/Å | | 1.34(2) | 1.372(3) | 1.355(5) | 1.353(3) | 1.355(8) |
| N-C (metformin, | 1.335 | | 1.344 | | 1.342 | |
| average.)/Å | | | | | | |
| N6-C15/Å | 1.461(4) | | 1.447(4) | | 1.456(3) | |
| N6-C14/Å | 1.462(4) | | 1.460(4) | | 1.459(3) | |
| C1-Au1-N1/° | | 83.0(6) | 81.16(10) | 85.7(1) | 87.93(9) | 89.5(3) |
| C1-Au1-N2/° | | | 94.65(10) | | 91.28(10) | |
| N2-Au1-N4/° | | | 87.94(10) | | 88.69(9) | |
| N1-Au1-N4/° | | | 96.25(9) | | 92.13(8) | |
| C1-Au1-Cl2/° | | 91.6(6) | | 90.8(1) | | 90.3(2) |
| N1-Au1-Cl1/° | | 95.4(4) | | 91.9(9) | | 92.0(2) |
| Cl1-Au1-Cl2/° | | 90.0(3) | | 91.6(4) | | 88.30(7) |
| N3-C12-N2/° | 122.6(3) | | 128.3(3) | | 128.7(2) | |
| N4-C13-N3/° | 116.7(3) | | 126.8(3) | | 126.0(2) | |
| N2-Au1-N1-C7/° | | | 4.7 | | 99.5 | |
| C12-N2-Au1-N1/° | | | 171.0 | | 117.7 | |

^{(a)}Hariharan M, Rajan SS, Srinivasan R. Structure of metformin hydrochloride. Acta Crystallogr, Sect C: Cryst Struct Common 1989; C45:911-13 ^{(b)}Fan D, Yang C-T, Ranford JD, Lee PF, Vittal JJ. Chemical and biological studies of the dichloro(2-phenylpyridine) gold(III) complex and its derivatives. Dalton Trans 2003:2680-5 ^{(c)}Cinellu MA, Zucca A, Stoccoro S, Minghetti G, Manassero M, Sansoni M. Synthesis and characterization of gold(III) adducts and cyclometallated derivatives with 2-substituted pyridines. Crystal structure of [Au{NC5H4(CMe2C6H4)-2}Cl2]. J Chern Soc, Dalton Trans 1995:2865-72
^{(d)}Fuchita Y, Ieda h, Tsunemune Y, Kinoshita-Nagaoka J, Kawano H. Synthesis, structure and reactivity of a new six-membered cycloaurated complex of 2-benzoylpyridine [AuCl2(pcp-C1, N)] [pcp = 2-(2-pyridylcarbonyl)phenyl]. Comparison with the cycloaurated complex derived from 2-benzylpyridine. J Chem Soc, Dalton Trans 1998:791-6

TABLE 3
Selected X-ray crystallographic data of 1met* and 2met.

| Complexes | 1met* | 2met |
|---|---|---|
| Formula | [C₁₅H₁₈N₆Au]Cl•CH₃OH | [C₁₆H₂₀N₆Au]PF₆ |
| Formula Weight | 546.81 | 638.32 |
| Temperature [K] | 100(2) | 100(2) |
| Wavelength [Å] | 0.71073 | 0.71073 |
| Crystal size [mm³] | 0.207 × 0.115 × 0.072 | 0.361 × 0.225 × 0.198 |
| Crystal system | Monoclinic | Monoclinic |
| Space group | P2₁/n | P2₁/c |
| a [Å] | 7.4277(3) | 8.2210(3) |
| b [Å] | 19.4868(7) | 18.7848(7) |
| c [Å] | 12.2140(5) | 13.3738(5) |
| α [°] | 90 | 90 |
| β [°] | 90.311(2) | 103.0470(10) |
| γ [°] | 90 | 90 |
| V [Å³] | 1767.85(12) | 2012.00(13) |
| Z | 4 | 4 |
| D_{c} [Mg/m³] | 2.054 | 2.107 |
| μ [mm⁻¹] | 8.491 | 7.462 |
| θ range [°] | 2.674 to 28.280 | 2.543 to 28.282 |
| Data/restraints/ | 4362/4/245 | 4975/4/285 |
| parameters | | |
| final R indices | R1 = 0.0196 | R1 = 0.0196 |
| [I > 2σ(I)] | wR2 = 0.0382 | wR2 = 0.0400 |
| R indices (all data) | R1 = 0.0269 | R1 = 0.0234 |
| | wR2 = 0.0398 | wR2 = 0.0407 |
| goodness-of-fit | 1.091 | 1.17 |
| on F² | | |
| peak/hole [e Å⁻³] | 0.567 and −1.178 | 0.474 and −1.476 |

R = Σ||Fₒ| − |F_{c}||/Σ|Fₒ|, wR2 = {Σ[w(Fₒ² − F_{c}²)²]/Σ[w(Fₒ²)²]}^{1/2}.
Goodness-of-fit (GOF) = {Σ[w(Fₒ² − F_{c}²)²]/(n − p)}^{1/2}, where n is the number of data and p is the number of parameters refined.

Complex 1met* is largely flat with both the guanide and cyclomethylated motifs adopting a square planar geometry around the Au centre. The angle subtended by the guanide plane and the cyclomethylated Au ring plane was 4.3°, indicating that both motifs are coplanar. Extensive short contacts with the chloride anion extends across the solid structure, with π-π stacking between closely packed molecules within the unit cell at distances of 3.37 Å apart. In contrast, 2met contained a pyridylbenzyl cyclometallated Au ligand that is bent out-of-plane due to its bridging methylene group. Both ligands bond Au centre in a square planar geometry that is expected to Au^{III}. The packing of the 2met is characterized by extensive short contacts with PF₆ anions with the cations spaced further apart at 3.61 Å.

### Solubility of Au^{III}-metformin and -phenformin Complexes

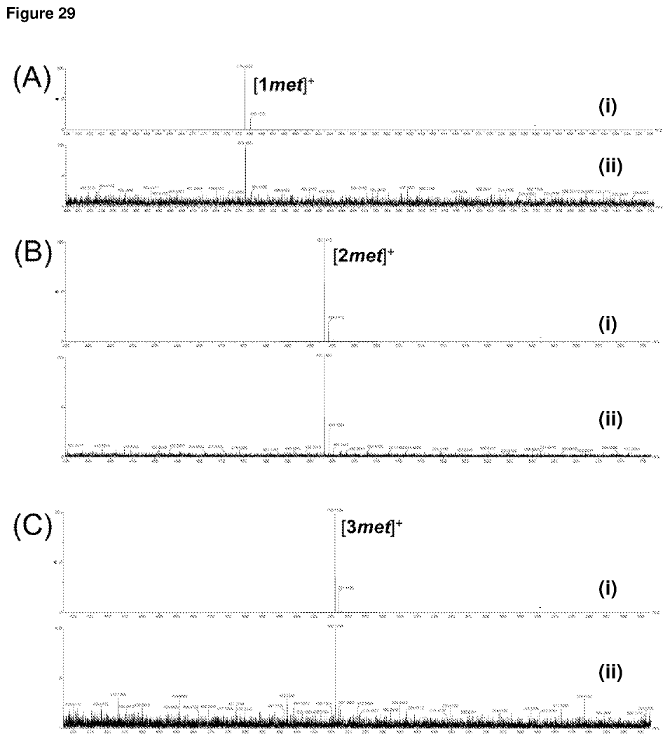

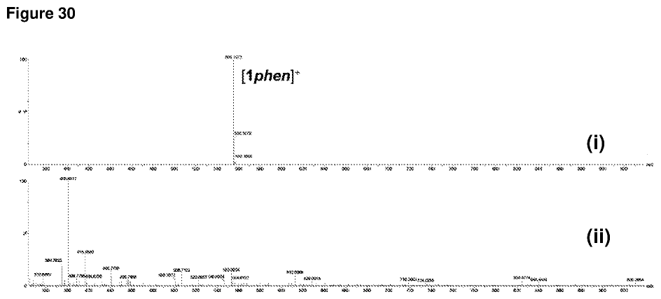

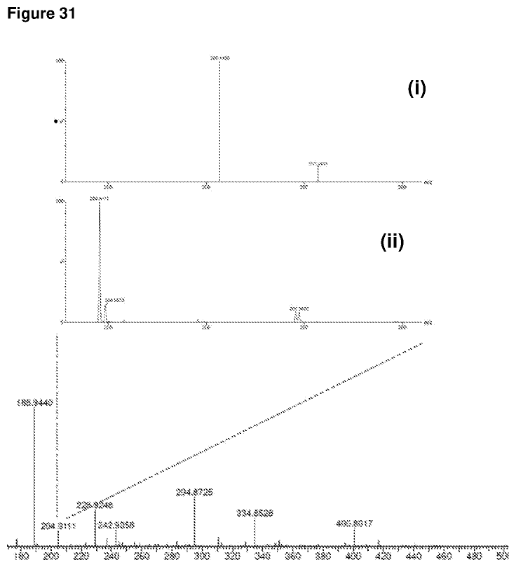

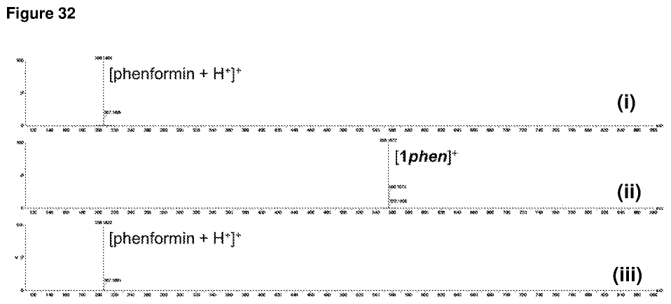

To determine the speciation of the Au^{III} complexes in aqueous solution, the compounds were incubated in ammonium carbonate buffer (pH 7.4) at 37° C. for 1, 3 and 24 hours and analyzed by high resolution ESI-MS. Compounds 1-3met were stable for 24 hours as evidenced by the detection of molecular ions [M]⁺ (Table 4 and FIG. 29), thus corroborating the NMR results. On the contrary, the [M]⁺ signal for 1phen was not detected after 1 hour incubation, indicating lower stability in comparison with the Au^{III}-metformin analogues (FIGS. 30 and 31). When 1phen was incubated in presence of GSH for the same time period, release of phenformin was detected at 206.1622 m/z (FIG. 32), which was not observed in the absence of GSH. Additionally, the stability of Au^{III} complexes in DMSO-d⁶ was confirmed using ¹H NMR spectroscopy over 10 days (FIGS. 12 to 14).

TABLE 4
Experimental (mₑₓₚ) and theoretical (mₜₕₑₒᵣ)
masses of the detected species during the stability
studies by high resolution mass spectrometry.

| Compound | Observed species | mₑₓₚ | mₜₕₑₒᵣ |
|---|---|---|---|
| 1met | [1met]⁺ | 479.1323 | 479.1259 |
| 2met | [2met]⁺ | 493.1758 | 493.1415 |
| 3met | [3met]⁺ | 506.1709 | 506.1129 |
| 1phen | Phenformin + H⁺ | 206.1406 | 206.1622 |

### Coordination of Metformin and Phenformin to an Au^{III} Center Resulted in Drastically Different Effects on the Antiproliferative Activity

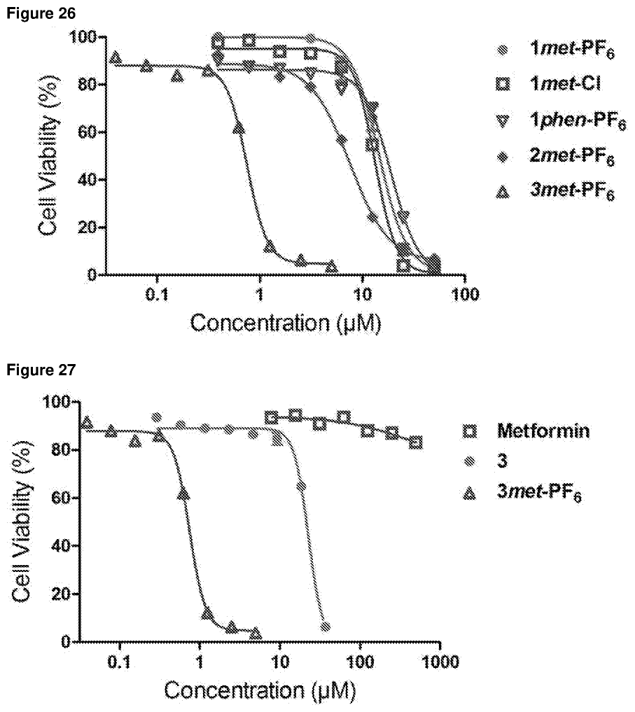

The in vitro anticancer activity of novel Au^{III} compounds was determined in human ovarian cancer cell lines (A2780 and A2780cisR) and human breast adenocarcinoma cell line (MDA-MB-231) by means of the colorimetric MTT assay with an exposure time of 72 hours. Their EC₅₀ values in comparison with uncoordinated metformin and phenformin, as well as cisplatin are given in Table 5 and concentration-effect curves are shown in FIGS. 26, 27 and 28.

Whereas metformin was devoid of cytotoxicity and phenformin was only marginally cytotoxic, the cytotoxicity of novel Au^{III} complexes was comparably high in all tested cell lines, in agreement with previously reported structurally similar Au^{III} complexes. In a cisplatin-sensitive A2780 cell line, these complexes were ≈3-10 times less cytotoxic than cisplatin; however, in cisplatin-resistant A2780cisR cells, novel complexes demonstrated up to 7-fold increase in cytotoxicity. The activity of Au^{III} precursors 1-3 decreased in the following rank order 1>2>3, whereas the activity of their metformin analogues followed the opposite trend: 3met>>2met>1met. With the exception of complex 1met, coordination of metformin resulted in the increase of the cytotoxicity of Au^{III}-metformin complexes, whereas coordination of phenformin negatively affected the cytotoxicity of 1phen in all tested cell lines. In particular, 3met was found to be more than 6000-fold more active than metformin.

The exchange of the counterion did not have a significant effect on the activity of the complexes; however, 1met* was excluded from further studies due to poor solubility in the biological media. Additionally, the compounds' toxicity in human ventricular cardiomyocytes AC10 in comparison with doxorubicin was assessed, which was severely cardiotoxic (Table 5). The heart toxicity of doxorubicin in AC10 cells was reflected by the IC₅₀ value 2.3±0.2 μM, whereas cisplatin and 3met were approximately 3-4-fold less toxic. All other Au^{III} complexes did not demonstrate any significant toxicity in heart cells. Similar results were observed upon assessment of the liver toxicity using mouse hepatocytes TAMH (Table 5).

TABLE 5
Cytotoxicity, TrxR inhibition and cellular accumulation of compounds of interest
(values are means ± standard errors of mean obtained from at least three independent
experiments).

| | | | | | | | | Cellular |
|---|---|---|---|---|---|---|---|---|
| | | | | | | | | accumulation,^{c} |
| | | | | | | | | nmol Au/mg |
| | EC₅₀ [μM]^{a} | | | | | | IC₅₀ [nM]^{b} | protein |
| Compound | A2780 | A2780cisR | RF^{d} | MDA-MB-231 | AC10 | TAMH | TrxR | MDA-MB-231 |
| 1 | 2.0 ± 0.4 | 4.0 ± 0.8 | 2 | 7.8 ± 1.7 | n.d. | n.d. | n.d.^{e} | 0.36 ± 0.10 |
| 1met* | 2.9 ± 0.5 | 4.8 ± 0.7 | 2 | 14 ± 2 | n.d. | n.d. | n.d. | n.d. |
| 1met | 1.8 ± 0.4 | 4.7 ± 0.6 | 3 | 11 ± 3 | 27 ± 2 | 16 ± 1 | 1.2 ± 0.5 | 0.30 ± 0.08 |
| 1phen | 4.4 ± 1.0 | 7.5 ± 1.2 | 2 | 14 ± 3 | 26 ± 2 | 16 ± 1 | 3.1 ± 1.5 | 0.31 ± 0.09 |
| 2 | 6.5 ± 0.8 | 7.2 ± 1.5 | 1 | 7.1 ± 0.4 | n.d. | n.d. | n.d. | 0.14 ± 0.03 |
| 2met | 2.3 ± 0.6 | 1.6 ± 0.5 | 1 | 7.0 ± 1.3 | >50 | 40 ± 5 | 0.55 ± 0.09 | 0.13 ± 0.02 |
| 3 | 8.6 ± 1.7 | 9.1 ± 2.9 | 1 | 19 ± 3 | n.d. | n.d. | n.d. | 0.14 ± 0.05 |
| 3met | 0.15 ± 0.03 | 0.53 ± 0.07 | 4 | 0.72 ± 0.08 | 7.4 ± 3.0 | 2.7 ± 0.7 | 1.3 ± 0.3 | 1.37 ± 0.48 |
| Metformin | >1000 | >1000 | — | >1000 | n.d. | n.d. | >5 mM | — |
| Phenfomin | 104 ± 21 | 246 ± 24 | 2 | 608 ± 23 | n.d. | n.d. | n.d. | — |
| Cisplatin | 0.60 ± 0.05 | 11 ± 2 | 18 | 27 ± 8 | 10 ± 2 | 52 ± 7 | n.d. | — |

^{a}50% effective concentrations in A2780 and A2780cisR (ovarian carcinoma), MDA-MB-231 (breast adenocarcinoma), AC10 (human ventricular cardiomyocytes) and TAMH (transforming growth factor-a transgenic mouse hepatocytes) determined by means of the MTT assay with the exposure time of 72 hours;
^{b}50% inhibitory concentrations for rat liver TrxR inhibition determined by means of DTNB reduction assay with the exposure time of 30 min;
^{c}cellular accumulation determined by ICP-MS after 24 hour exposure to MDA-MB-231 cells at concentration of 1.4 μM;
^{d}Resistance Factor (RF) was determined as IC_{50 (A2780cisR)}/IC_{50 (A2780)};
^{e}n.d.-not determined.

### Intracellular Ligand Dynamics of Au^{III}-Metformin and -Phenformin Complexes

In the presence of biologically relevant thiols, such as glutathione (GSH), Au(III)-metformin complexes undergo irreversible redox reactions. The mechanism of activation may be described as competition between ligands substitution and redox reactions; however, the redox potential of these reactions is high and cannot be accessed in living cells. Therefore, it is assumed that activation of the complexes occurs via ligand exchange rather than electrochemically.

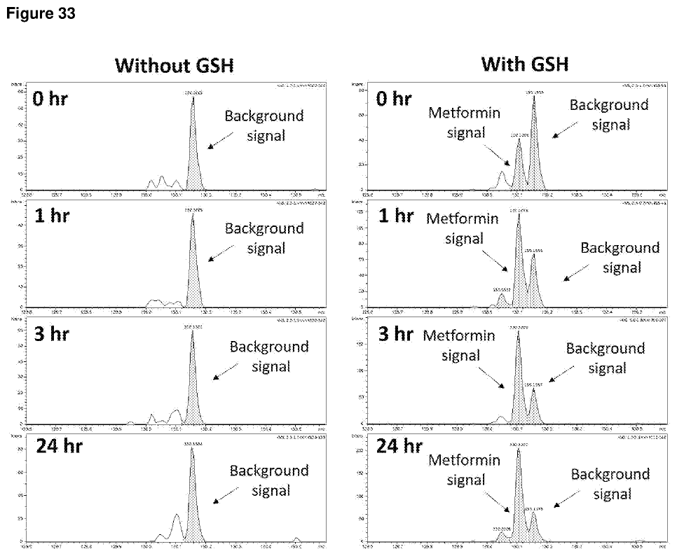

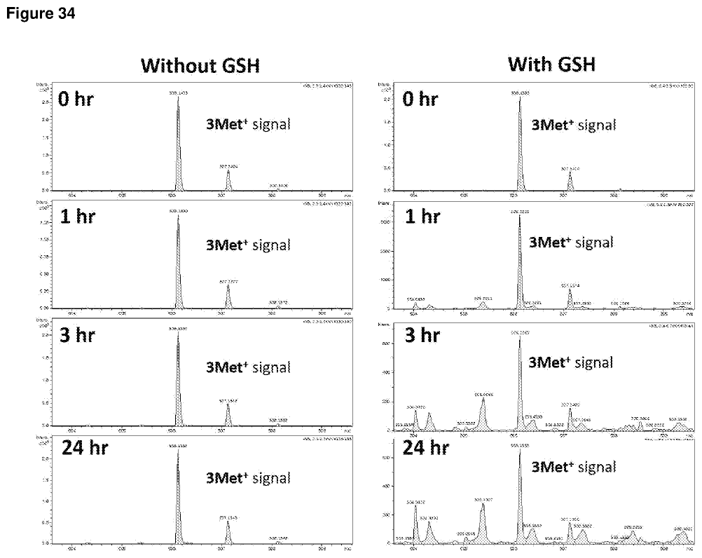

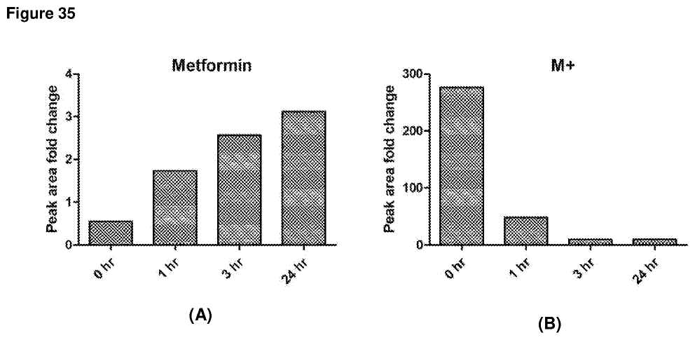

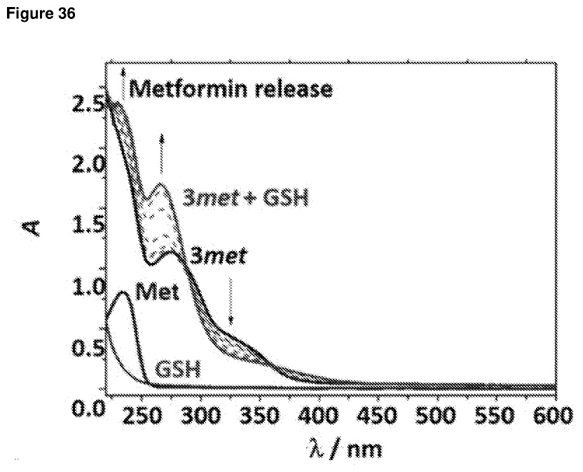

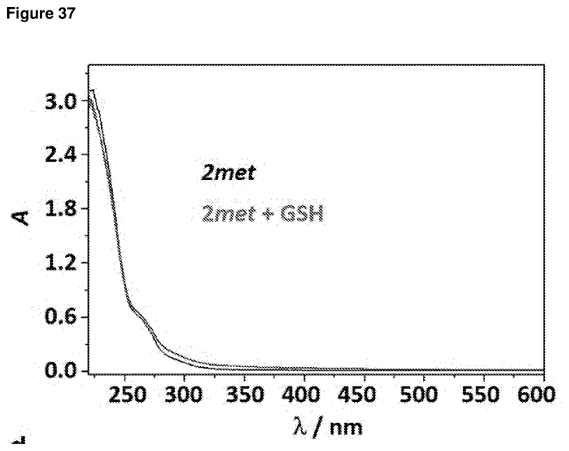

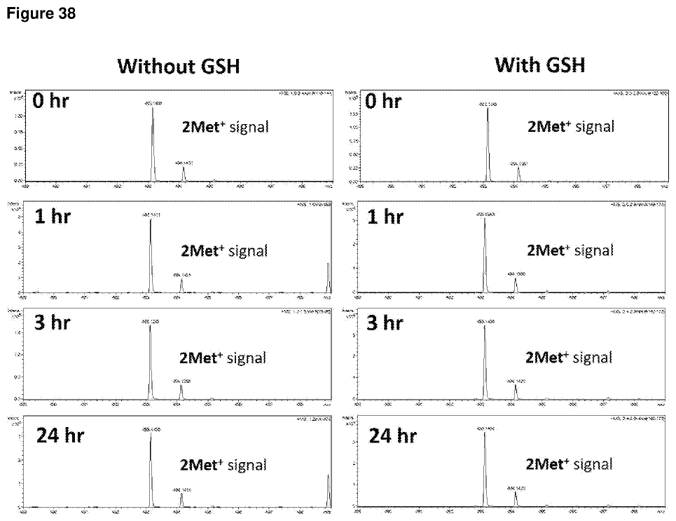

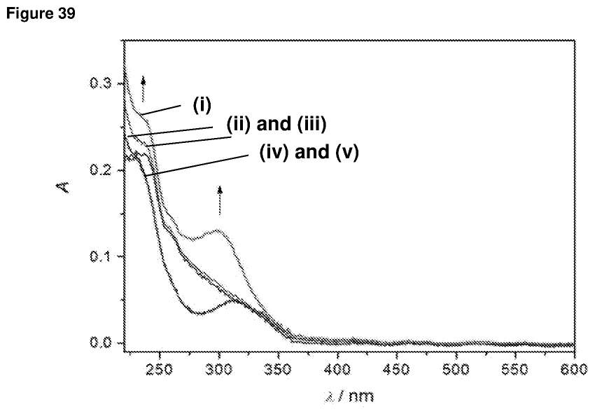

Upon interaction with intracellular glutathinone (GSH), Au(III) complex 3met was shown to release free metformin. The release of metformin ligand was monitored by high-resolution mass spectrometry for 24 hours. It was shown that the peak area corresponding to metformin signal (m/z 130.1078, FIG. 33 and FIG. 35(A)) increased more than 5-fold after 3 hours, whereas the peak area corresponding to 3met⁺ signal (m/z 506.1358, FIG. 34 and FIG. 35(B)) decreased more than 27-fold. Intriguingly, the reactivity of Au^{III}-metformin complexes towards GSH was drastically different despite their similar structures. 3met demonstrated time-dependent release of metformin characterized by evident optical changes in UV-vis spectrum and appearance of the new peak at 235 nm corresponding to free metformin (FIG. 36). In contrast to 3met, complex, 2met was stable both in the absence and presence of GSH, which may explain its significantly lower cytotoxicity in cancer cells (FIGS. 37 and 38) while 1met demonstrated metformin release only upon heating (FIG. 39). It has been previously shown that an Au^{III} complex featuring 2-benzoylpyridine scaffold efficiently arylated GSH via a reductive elimination process, in agreement with enhanced reactivity of 3met.

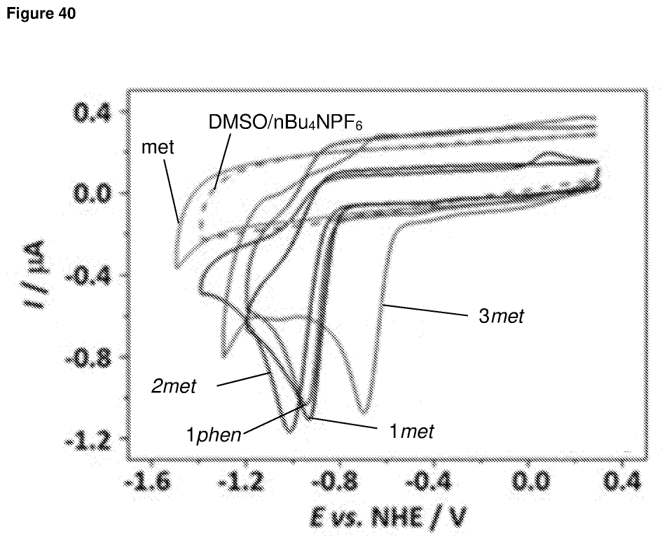

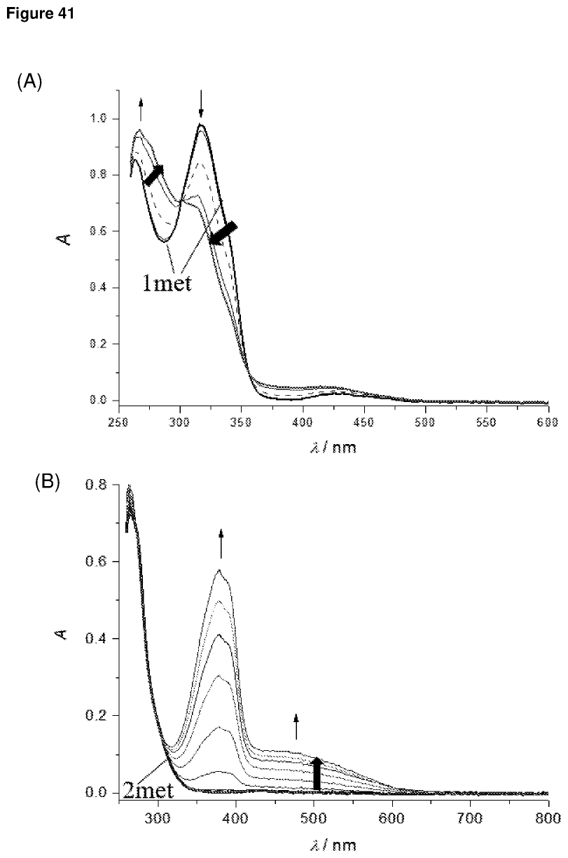

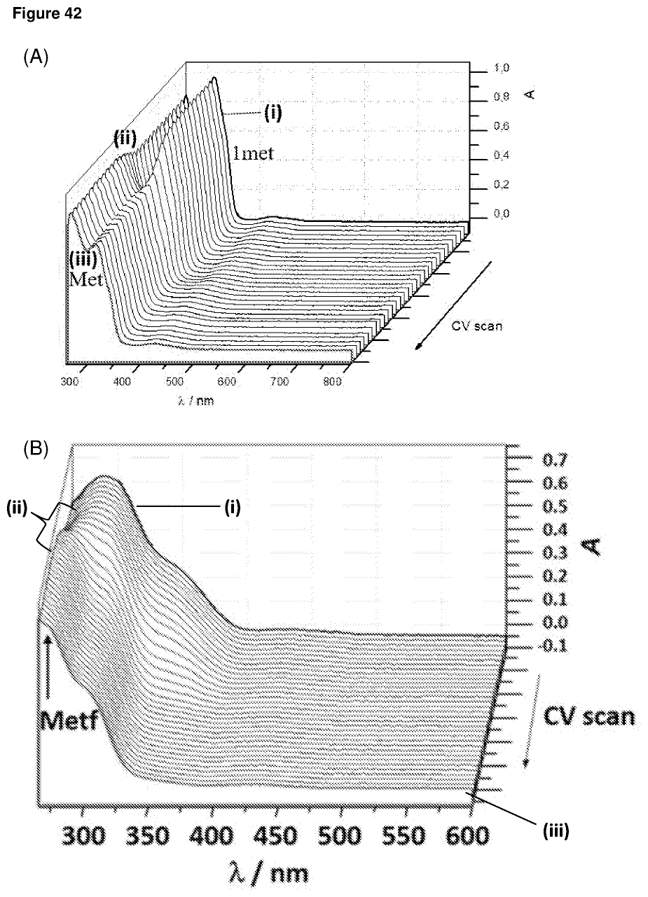

To determine whether the release of metformin occurred as a result of electrochemical reduction of Au^{III}, cyclic voltammetry experiments were performed in DMSO and aqueous solution (FIG. 40). While uncoordinated metformin did not show any redox activity, the cyclic voltammograms of 1met, 2met, 3met and 1phen demonstrated a reduction wave in the cathodic region at −0.6 to −1.1 V (vs. NHE), corresponding to an irreversible reduction of Au^{III} to Au^{I}. However, the redox potentials were outside the accessible biological window, indicating that direct reduction of Au^{III} in cancer cells was unlikely. Subsequently, cyclic voltammetry measurements were coupled with UV-vis in a spectroelectrochemical cell, which revealed that cathodic reduction of Au^{III} in 1met and 3met but not 2met, was associated with the appearance of new transitions in the region between 350 and 600 nm (FIGS. 41 and 42). Taken together, the interaction of Au^{III} complexes with GSH might be considered as a competition between reduction and ligand substitution. However, it is proposed that ligand substitution occurred prior to reduction. In the case of 3met, it is hypothesized that further gold-templated C—S cross coupling also occurs.

### Au^{III}-Metformin and Phenformin Complexes Inhibit TrxR Enzyme in a Nanomolar Concentration Range in Contrast to Metformin

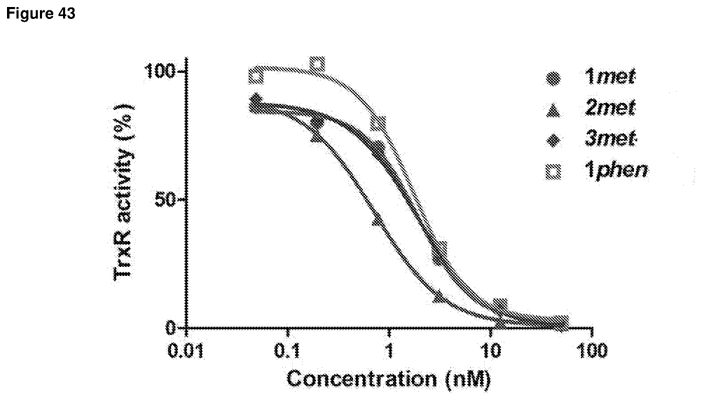

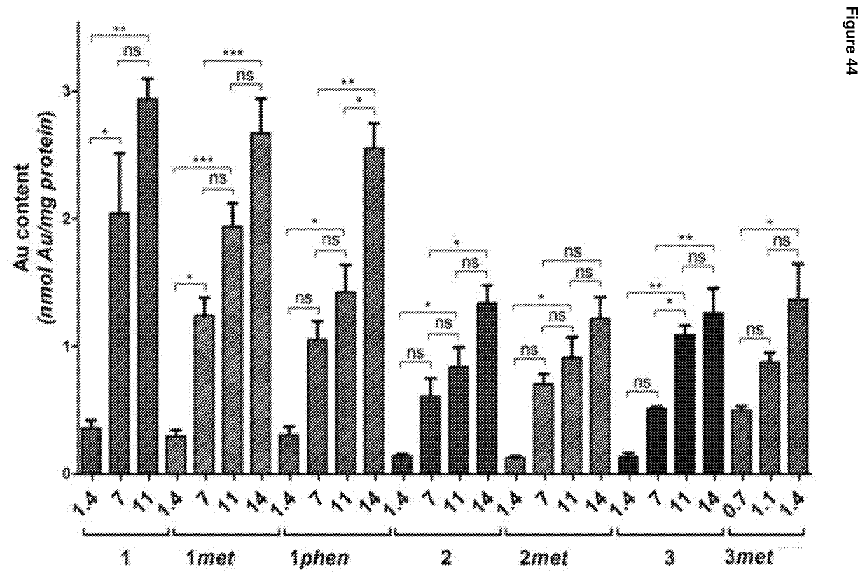

Since the mechanism of Au^{III} complexes in cancer cells could involve the inhibition of TrxR enzyme, the TrxR-inhibitory potential of 1-3met, 1phen and metformin was investigated by the colorimetric DNTB (dithiobisnitrobenzoic acid) reduction assay using rat liver TrxR. TrxR reacts with DNTB in presence of NADPH, resulting in the formation of a colored product, which can be detected photometrically. Compounds of interest at six different concentrations were incubated with the isolated rat liver TrxR enzyme in the presence of NADPH for 30 minutes, followed by 2 minutes incubation with the DNTB reagent. Subsequently, the absorbance at 412 nm was measured for 30 minutes and the EC₅₀ values were derived from concentration-effect curves (Table 5 and FIG. 43). All tested complexes demonstrated comparable nanomolar inhibitory activity (≈1-3 nM) against the seleno-enzyme with the highest effect observed for 2met (0.55±0.09 nM). In contrast, uncoordinated metformin did not show any inhibitory potential up to 5 mM; therefore, TrxR-inhibitory potential of Au^{III}-metformin complexes was attributed to the Au^{III} moiety. Inhibition of mitochondrial TrxR may trigger various antimitochondrial effects, reflected by the changes in oxidative stress and membrane depolarization.

### Improved Anticancer Activity of the Lead Complex 3met Correlates with its More Efficient Intracellular Accumulation

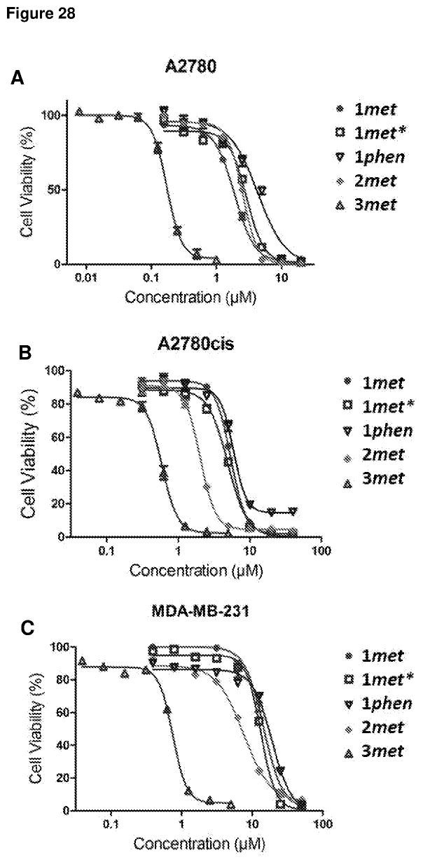

The Au^{III} complexes were tested against the aggressive poorly-differentiated triple negative breast cancer (TNBC) cell line, MDA-MB-231, as well as other human cancer cell lines, and exhibited high cytotoxicities in all cases (Table 5, FIG. 28). In contrast, metformin was devoid of cytotoxicity, while phenformin was only marginally cytotoxic. In keeping with reduced stability, 1phen was the least cytotoxic representative of this series. Additionally, the compounds' toxicity was assessed in human ventricular cardiomyocytes in comparison with doxorubicin, a topoisomerase II inhibitor used to treat TNBC but which suffers from severe cardiotoxicities, as a control (Table 5). The heart toxicity of doxorubicin in human ventricular cardiomyocytes (AC10) cells was reflected by the IC₅₀ value 2.3±0.2 μM, whereas cisplatin and 3met were approximately 3-4-fold less toxic. All other Au^{III} complexes were only marginally toxic or non-toxic at all. Similar results were observed upon assessment of the liver toxicity using mouse hepatocytes (TAMH) (Table 5). It should be noted that 3met, while being more toxic than other structurally similar complexes, demonstrated 4-fold selectivity to liver cells over resistant MDA-MB-231 cancer cells and 18-fold selectivity over cisplatin sensitive A2780 cancer cells.

The differences in cytotoxicity of Au^{III} complexes may be related to their intracellular accumulation. Therefore, the intracellular Au content in MDA-MB-231 cells was determined by ICP-MS upon exposure to increasing concentrations of compounds for 24 hours (Table 5 and FIG. 44). All complexes demonstrated concentration-dependent cellular accumulation with the highest accumulation for 3met (*** p<0.001, one-way ANOVA test with Dunnett's post hoc analysis), in agreement with its highest cytotoxicity. The accumulation of 3met was ≈10 times higher than that of 2met, corresponding to its ≈10 times higher activity. The cellular accumulation of other complexes was comparable.

### 3met Caused Bioenergetic Crisis in Cancer Cells Characterized by Altered Mitochondrial Respiration and Glycolysis

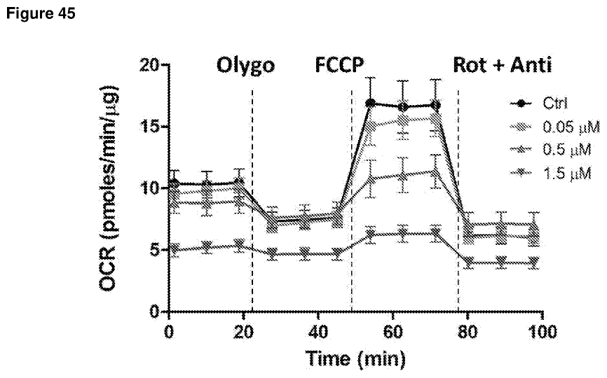

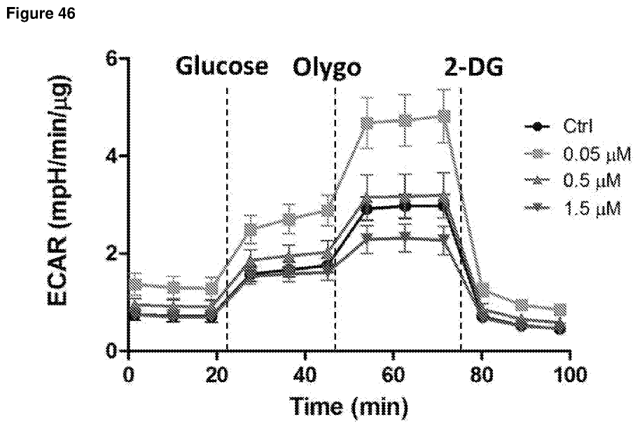

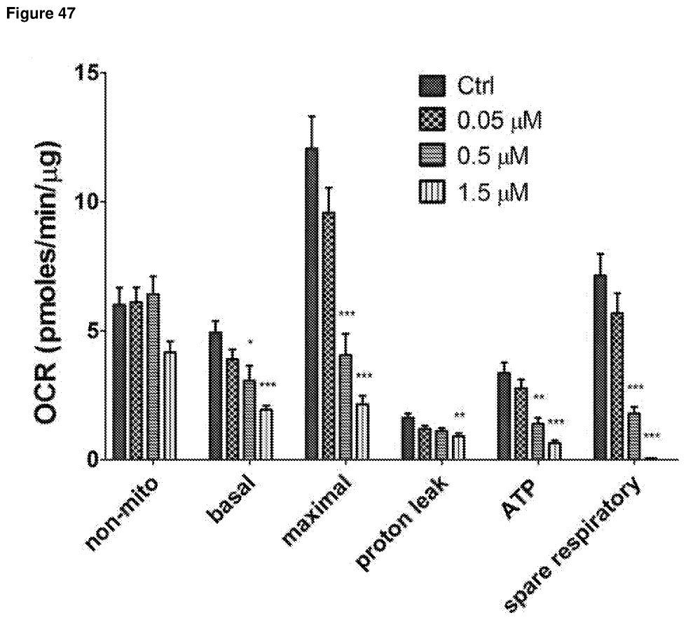

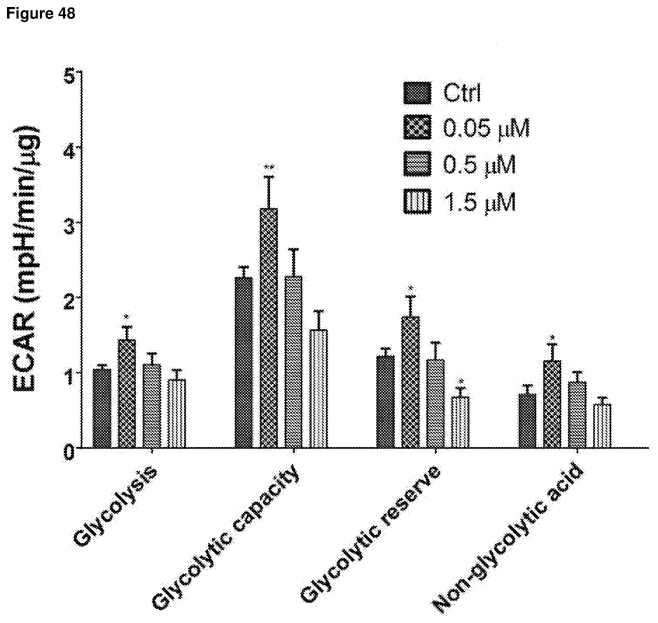

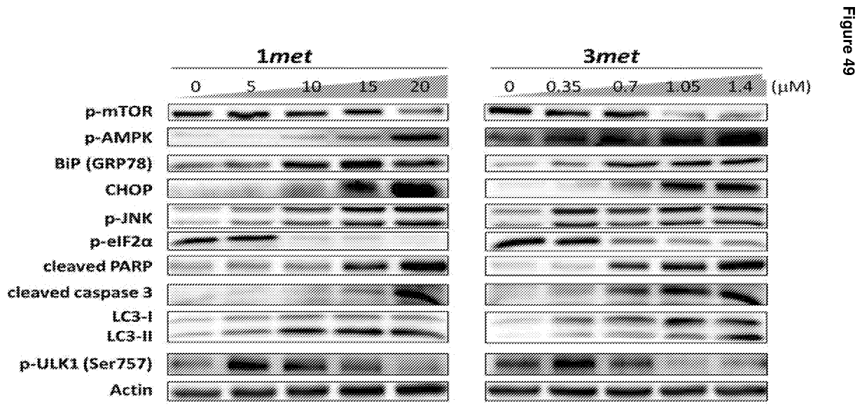

Therapeutic effects of metformin are related to the alterations in cellular mitochondrial activity. As the inner mitochondrial membrane contains the respiratory enzymes necessary for oxidative phosphorylation (OXPHOS), its depolarization leads to defective mitochondrial respiration and energy metabolism. The effects of 3met on mitochondrial respiration of highly resistant MDA-MB-231 cells were therefore investigated (FIGS. 45 and 47) by measuring oxygen consumption rate (OCR) using Seahorse MitoStress assay. Cancer cells were treated with increasing concentrations of compounds of interest for 24 hours and OCR was measured every few minutes before and after the addition of the respiratory modulators, namely oligomycin, carbonyl cyanide 4-(trifluoromethoxy) phenylhydrazone (FCCP), rotenone and antimycin A. 3met demonstrated dose-dependent progressive decrease of all mitochondrial bioenergetic parameters, indicating inhibition of mitochondrial processes and loss of mitochondrial mass, similar to other mitochondria-targeting metal-based complexes. In contrast, non-mitochondrial respiration was not significantly inhibited (FIG. 35). When mitochondria were prevented from ATP generation, the cells were prompted to take up more glucose, resulting in lower blood glucose levels. Since the loss of ATP in cancer cells was counterbalanced by an increased glycolytic rate, the glycolytic function of MDA-MB-231 cells after 24 hour drug incubation was analyzed by measuring extracellular acidification rate (ECAR) using Seahorse Glycolysis stress test. ECAR was measured every few minutes before and after addition of glucose, oligomycin and 2-deoxyglycose (2-DG) (FIGS. 46 and 48). When cancer cells were treated with low concentrations of 3met (0.05 μM) for 24 hours, the significant increase of all glycolytic parameters was observed. On the contrary, a 24 hour exposure of MDA-MB-231 cells to higher concentrations of 3met at 1.5 μM resulted in the significantly declined glycolytic reserve. These results indicate the attempts of cancer cells to confer a survival advantage in presence of 3met by greater compensatory increase in aerobic glycolysis.

### Effects of Au^{III}-Metformin Complexes on Protein Kinases Involved in Metabolic Signaling

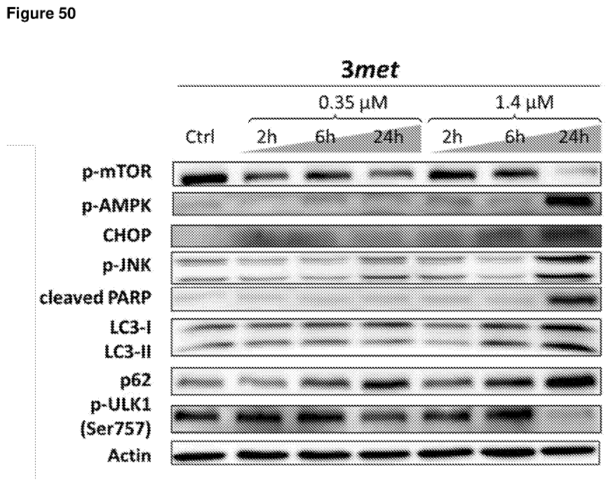

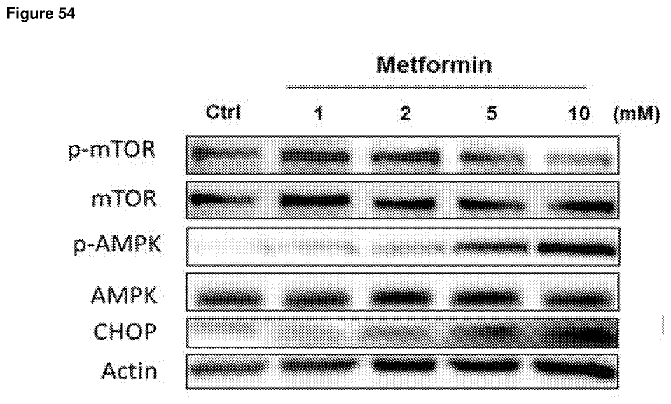

Metabolic actions of metformin in cancer cells can occur both in AMPK-dependent or independent manner. To better understand the signaling events underlying the metabolic action of Au^{III}-metformin complexes, their effects on AMPK-mTOR energy signaling pathway were investigated. AMPK regulates the energetic balance at the whole body level responding to external nutrient and growth input and mTOR is a downstream target of AMPK, as well as a key energy sensor involved in the regulation of ribosome biosynthesis and ATP production. mTOR undergoes phosphorylation when growth conditions are favourable; however, unlike mTOR, the phosphorylation of AMPK occurs during fuel deficiency. The concentration-dependent behavior of 1met and 3met was assessed upon treatment of MDA-MB-231 cells with increasing concentrations (corresponding to 0.5×, 1×, 1.5× and 2×EC₅₀ values, respectively) of 1met and 3met for 24 hours in comparison with metformin (FIGS. 49 and 54). It was shown that lmet and 3met caused dose-dependent increase of AMPK phosphorylation and decrease of mTOR phosphorylation, similar to high concentrations of uncoordinated metformin (FIG. 52). Additionally, time-dependent behavior of 3met was studied upon incubation of MDA-MB-231 with 3met at concentrations corresponding to its 0.5× and 2×EC₅₀ values for 2, 6 and 24 hours (FIG. 50) and time-dependent increase of p-AMPK and decrease of p-mTOR was observed. The Western blot results indicated the activation of AMPK followed by mTOR inhibition, thereby supporting the observed effects of the complexes on the mitochondrial respiration. The inhibition of cancer metabolism induced by 3met might be related to the inhibition of kinases, involved in the energy regulation processes.

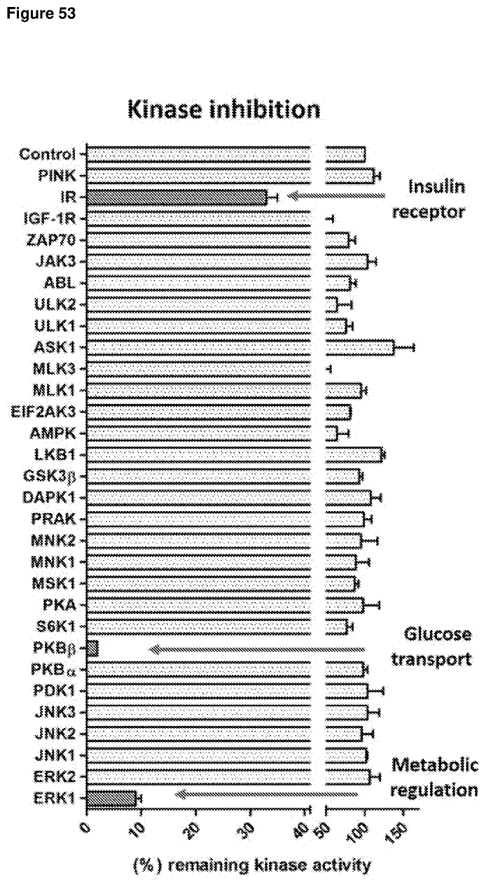

Subsequently, the effects of 3met on the residual in vitro activity of 30metabolically-relevant kinases was determined using radioactive ³³P-ATP filter-binding assay (FIG. 53). The analysis revealed that 3met was a relatively specific inhibitor, with the most significant inhibition observed upon incubation of 3met with protein kinase B beta (PKBβ, 100%), extracellular signal-regulated kinase 1 (ERK1, 90%), and insulin receptor (IR, 70%) kinases, which play key roles in the metabolic function of cancer cells. Importantly, metformin was also reported to be involved in PKB, ERK and IR signaling.

### Au^{III}-Metformin Complexes Activated Unfolded Pprotein Response (UPR) in Response to Endoplasmic Reticulum (ER) Stress

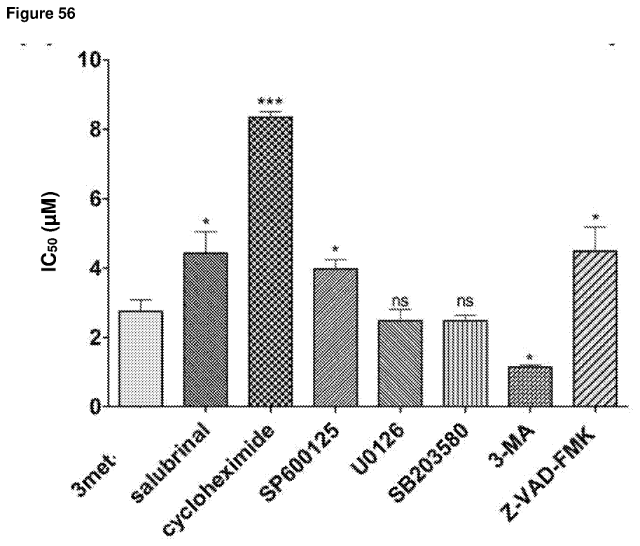

The major role in restoring normal cellular function under stressful conditions is mediated by unfolded protein response (UPR) and autophagy, which are activated in response to the accumulation of unfolded or misfolded proteins in the endoplasmic reticulum (ER). To gain further insights into the mechanism of action of Au^{III}-metformin complexes, the expression of ER stress-related markers, namely, binding immunoglobulin protein (BiP) and C/EBP homologous protein (CHOP), as well as phosphorylation of elF2α (p-elF2α), were monitored by means of Western Blotting. The dose-dependent behaviour of 1met and 3met was assessed upon treatment of MDA-MB-231 cells with increasing concentrations (corresponding to 0.5×, 1×, 1.5× and 2×EC₅₀ values, respectively) of 1met and 3met for 24 hours. It was demonstrated that Au^{III} complexes caused dose-dependent increase of BiP and CHOP expression, while simultaneously decreasing the expression of p-elF2α, indicating cellular response to drug-induced ER stress (FIG. 49). When MDA-MB-231 cells were treated with 3met (at concentrations corresponding to 0.5 and 2EC₅₀ values) for 2, 6 and 24 hours, time-dependent increase of CHOP was observed (FIG. 50). To investigate the role of elF2α and protein synthesis in the mode of action of 3met, cancer cells were co-treated with 3met and sub-toxic concentrations of salubrinal (selective inhibitor of elF2α dephosphorylation, 10 μM) or cycloheximide (CHX, protein synthesis inhibitor, 12.5 μM) for 24 hours (FIG. 56). Addition of salubrinal caused a ≈2-fold decrease of 3met activity, whereas CHX abrogated 3met-induced cell death by ≈3.5 times.

### 3met-Induced Cell Death is Associated with Activation of JNK

Prolonged ER stress can result in the phosphorylation of c-Jun N-terminal kinase (JNK) and release of pro-apoptotic proteins, thereby facilitating JNK-mediated apoptosis. A dose-dependent study revealed that exposure of MDA-MB-231 cells to 1met and 3met resulted in JNK phosphorylation already at 0.5 EC₅₀ drug concentrations and the event became apparent after 24 hours of exposure (FIGS. 49 and 50). To assess the role and specificity of JNK in 3met-mediated cell death, MDA-MB-231 cells were co-treated with a combination of 3met and a panel of inhibitors, including those of JNK kinase (SP600125, 20 μM), p38 MAPK kinase (SB203580, 20 μM) and MEK1/2 kinase (U0126, 20 μM). The activity of 3met was abrogated in the presence of SP600125 (by ≈1.7 fold), but not in the presence of SB203580 and U0126, indicating the role of JNK in the 3met-mediated cell death (FIG. 44).

### 3met Activated Pro-Survival Autophagy Signaling and Impaired Autophagic Flux

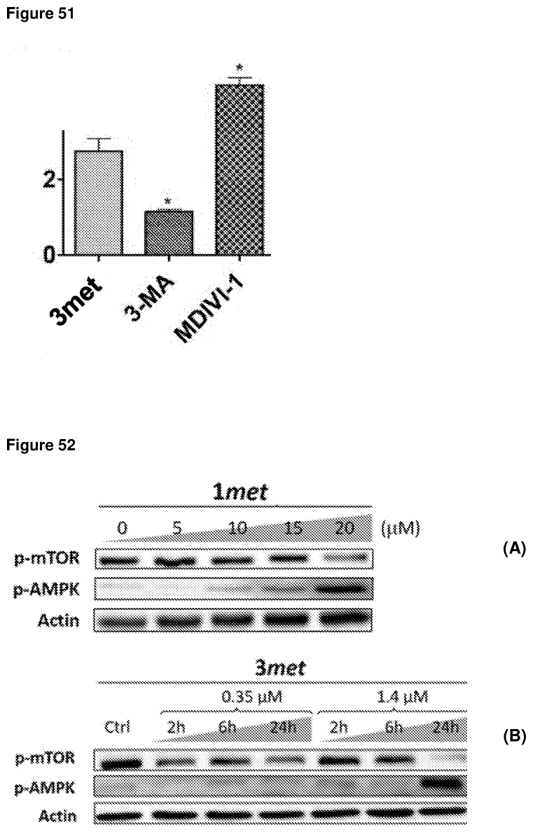

Au^{I} and Au^{III} complexes commonly induce UPR and ER stress; however, autophagy-inducing Au complexes are relatively rare. Because ER stress may result in the activation of pro-survival or pro-death autophagy signaling, it was sought to determine whether 3met-induced cell death was associated with the activation of the autophagy program. Autophagy ensures the self-removal of cell's own faulty material. One of the hallmarks of autophagy was the conversion of cytosolic LC3-I to autophagosome-bound LC3-II which was monitored by Western Blotting. FIGS. 39 and 40 showed that treatment of MDA-MB-231 cells with 1met and 3met significantly enhanced the expression of LC3-II in a concentration- and time-dependent manner, indicating the activation of the autophagy program. When the cytotoxicity of 3met was tested in presence of autophagy inhibitor 3-methyladenine (3-MA), it increased more than 2 times, indicating the pro-survival role of autophagy processes (FIG. 51).

In order to assess the role of AMPK-mTOR signalling in the regulation of autophagy, the effects of Au^{III} complexes on Ulk1 protein phosphorylation was determined. Under starvation conditions and inhibition of mTOR, Ulk1 is rapidly dephosphorylated at Ser757 site. As can be seen in FIGS. 49 and 50, whereas at low concentrations (0.5 and 1EC₅₀) of 1met and 3met enhanced phosphorylation of Ulk1 was observed, higher concentrations (2EC₅₀) of the drugs and long incubation time (24 hours) led to the marked dephosphorylation of Ulk1 at Ser757 site, coinciding with the activation of AMPK.

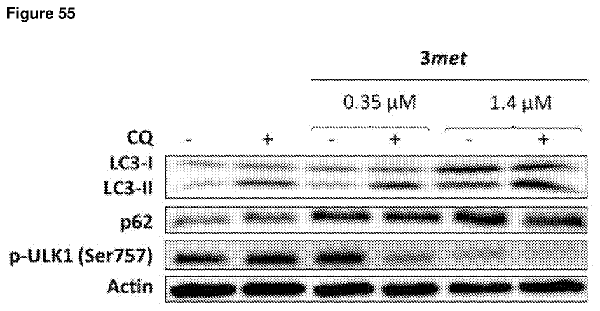

During the process of autophagy, cellular contents are engulfed by double-membrane vesicles called autophagosomes which undergo fusion with lysosomes to become autolysosomes. Subsequently, lysosomal enzymes in the autolysosomes effectively degrade the cargo inside autophagosomes. The increase of LC3-II/LC3-I ratio as a result of 1met and 3met treatment might be either an outcome of the elevated autophagic activity or impaired autophagic flux characterized by the inhibition of protein degradation and the accumulation of autophagosomes. These possibilities can be resolved by assessment of LC3-II levels in presence of chloroquine (CQ), which blocks the fusion of the autophagosome with the lysosome, thereby preventing degradation of LC3-II in autolysosomes. CQ was added 1 hour before the cell harvest to inhibit lysosomal activity, resulting in the accumulation of LC3-II protein. When MDA-MB-231 cells were treated with the combination of 3met with CQ, further increase of LC3-II levels was observed. Additionally, the expression of ubiquitine-associated protein p62, which directly binds to LC3 and is commonly used to monitor autophagic flux, was analyzed. Upon treatment with 3met, the increase of p62 levels was observed in accordance with accumulation of autophagosomes and no further increase was observed in combination with CQ (FIG. 55); however, Ulk1 dephosphorylation was significantly enhanced when cells were treated together with CQ, further supporting autophagy flux impairment. In order to distinguish between pro-survival or pro-death autophagy signalling, MDA-MB-231 cells were co-treated with 3met and autophagy inhibitor, 3-methyladenine (3-MA, 2 mM) for 24 hours. The activity of 3met in presence of 3-MA increased more than 2 times, indicating pro-survival role of autophagy processes. The results demonstrated that 3met inhibited protein degradation and the accumulation of autophagosomes, leading to the impairment of pro-survival autophagic flux, which was distinctly different mechanistically from other structurally similar cyclometallated Au^{III}—C,N complexes (FIG. 56).

It was investigated whether drug-induced mitochondrial dysfunction activated the process of mitophagy, which aims to restore cellular mitochondrial function by clearing defective mitochondria. Selective degradation of mitochondria occurs by increasing mitochondrial fission. When MDA-MB-231 cells were treated with 3met in combination with mitochondrial fission inhibitor MDIVI-1, its cytotoxicity significantly decreased, clearly indicating the role of mitophagy in the mechanism of 3met (FIG. 51). Notably, uncoordinated metformin was also shown to regulate mitophagy in vitro and in patients.

### Au^{III}-Metformin and Phenformin Complexes Induced Apoptotic Cell Death

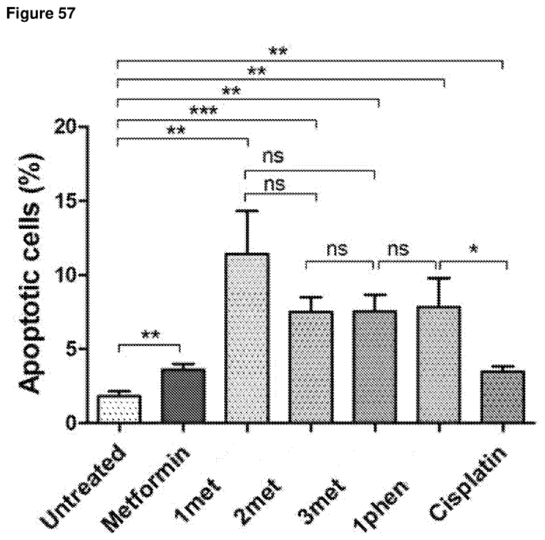

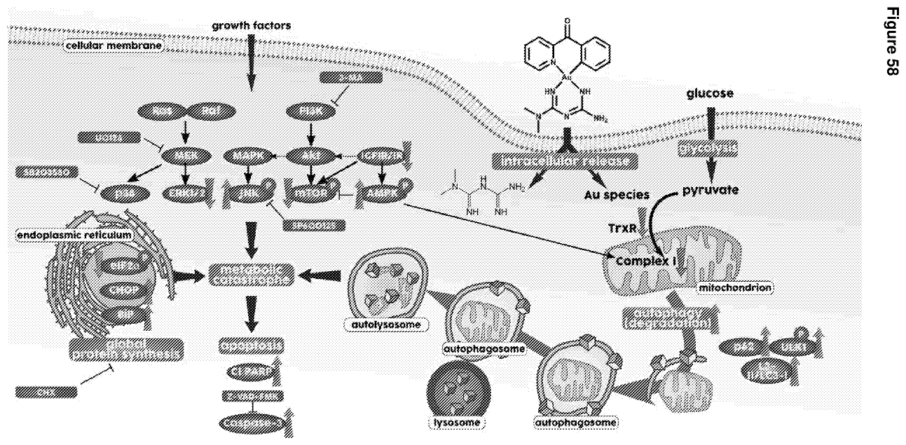

To determine if anticancer effects of novel Au^{III} complexes were due to apoptosis, an Annexin V/PI assay was performed in MDA-MB-231 cells. The early stages of apoptosis are characterized by the translocation of phosphatidyl serine (PS) from the inner leaflet of plasma membrane to the outer cell surface. The event of PS translocation can be captured by fluorochrome-labelled Annexin V, which reacts with the membrane PS residues. Addition of propidium iodide (PI) allows discrimination of the cells with damaged cellular membrane integrity. MDA-MB-231 cells were treated with the compounds of interest at equipotent concentrations, corresponding to their IC₅₀ values (FIG. 57). Complexes 1-3met caused marked increase in apoptotic cell population and variation of the cyclometalated fragment did not affect the ability of the complexes to induce apoptosis (p>0.05) when treated at equipotent concentrations. Similarly, no changes in apoptotic population were observed when metformin was replaced by phenformin in complex 1phen. However, when cells were treated with complex 1, the percentage of apoptotic population was significantly higher (***p<0.001) than for all other complexes. Next, the compounds of interest were compared to cisplatin and metformin. Although both compounds induced apoptosis in MDA-MB-231 cells, the percentage of apoptotic cells was markedly lower than for all tested Au^{III} complexes. Subsequently, the effects of 1met and 3met on apoptotic markers, namely cleaved poly(ADP-ribose)polymerase-1 (PARP) and cleaved caspase-3 were investigated and compared with the cytotoxicity of 3met in presence or absence of poly-caspase inhibitor Z-VAD-FMK (FIGS. 47 and 49). PARP cleavage is commonly accepted as an indicator of apoptotic cell death and caspase-3 is one of the key mediators of mitochondrial apoptosis. As can be seen in FIGS. 49 and 50, the cytotoxicity of 3met significantly decreased when cells were co-treated with poly-caspase inhibitor Z-VAD-FMK while compounds of interest demonstrated dose- and time-dependent PARP cleavage and increase expression of cleaved caspase 3, suggesting that Au^{III}-metformin complexes exerted mitochondrial caspase 3-dependent apoptosis in vitro.

### Discussion on In Vitro Activity

Novel Au^{III}-metformin complexes exhibited excellent cytotoxicities in a micromolar to nanomolar concentration range in all tested cell lines, including highly resistant triple negative breast cancer MDA-MB-231 cells (Table 2 and FIGS. 26 and 27). In particular, the lead complex 3met displayed nanomolar activities and was ≈4-, 21- and 28-fold more active than cisplatin in A2780, A2780cis and MDA-MB-231 cells, respectively. The compound was also markedly more active than its cyclometalated precursor 3 and demonstrated impressive 6500-fold increase in cytotoxicity compared to uncoordinated metformin (FIG. 27). The improved anticancer activity of 3met was linked to its significantly higher intracellular uptake, in agreement with the initial design (FIG. 44). As opposed to metformin, coordination of phenformin to an Au^{III} center resulted in a reduction of anticancer activity of 1phen, which was explained by the reduced stability of Au^{III}-phenformin complexes.

Although various Au complexes and nanoparticles showed promising anticancer potential, their clinical development has been hampered by their high toxicity. In particular, Au biodistribution in mice organs was characterized by high Au concentration in liver in agreement with its excretory function and unusually high Au levels in heart. It was hypothesized that coordination of metformin to an Au^{III} center would result in a reduced toxicity, since metabolic effects of metformin decrease radiation-induced cardiac toxicity risk and even reduce the incidence of heart failure in patients. Metformin also improves liver function in patients with liver diseases. As expected, Au^{III}-metformin and phenformin complexes demonstrated only marginal toxicity to liver and heart or were not toxic at all. In particular, 3met demonstrated 4- and 10-fold selectivity to liver and heart cells, respectively, over resistant MDA-MB-231 cancer cells and 18- and 49-fold selectivity over sensitive A2780 cancer cells (Table 2).

Metformin can directly affect cancer cell metabolism both in vitro and in vivo. However, its anticancer metabolic effects were observed at a high dose of 500 mg/kg/d, which is at least a 25-times higher dose than the conventional antidiabetic dose in humans and might not be relevant in a clinical setting. In this work, the lead Au^{III}-metformin complex 3met significantly inhibited mitochondrial respiration of triple-negative breast cancer cells at concentrations as low as 1.5 μM (FIGS. 45 and 47). The increase in AMPK activation caused by 3met occurred at ≈14000-fold lower concentration of 3met than metformin (FIGS. 49 and 54). The enhanced mitochondria-targeting potential might be related to the synergistic action of metformin and Au^{III} species, which were repeatedly reported to target mitochondrial cell death pathways. Au complexes efficiently inhibit TrxR enzyme, leading to the disruption of mitochondrial functions. It was demonstrated that novel Au^{III}-metformin and phenformin complexes inhibited TrxR at nanomolar concentration range, whereas metformin did not show any inhibitory potential up to 5 mM (FIG. 43).

Intriguingly, the effects of 3met on the glycolytic function of MDA-MB-231 cells were concentration-dependent. Cancer cells displayed elevated aerobic glycolysis upon exposure to low concentrations of 3met (0.05 μM) while at higher concentrations (1.5 μM), their glycolytic functions declined (FIGS. 46 and 48). These results indicated the attempts of cancer cells to confer a survival advantage in presence of 3met by greater compensatory increase in aerobic glycolysis. However, prolonged exposure to high concentrations of 3met resulted in a severe energetic crisis leading to the cell death.

Besides metabolic reprogramming, cancer cells develop various adaptive responses which provided them a survival advantage under stressful conditions. These responses are closely linked to each other and their crosstalk affect the decision of cancer cells whether to live or die. The major role in restoring cellular homeostasis is played by two pro-survival programs, namely autophagy and UPR, which mediate the switch between cellular outcomes by fine-tuning cancer cell signals. UPR is activated in response to the accumulation of unfolded or misfolded proteins in the ER, whereas autophagy is characterized by lysosomal degradation of the cells' own material to maintain cellular energy balance. 1met and 3met activated autophagic signaling characterized by increased p62 and autophagosome-bound LC3-II expression, as well as marked dephosphorylation of Ulk1 at Ser757 site (FIG. 49).

3met was able to induce pro-survival UPR activation in MDA-MB-231 cells characterized by dose- and time-dependent activation of the key UPR folding chaperone, binding immunoglobulin protein (BiP) (FIG. 49 (right side) and 52). However, decreased phosphorylation of p-elF2α, increased phosphorylation of c-Jun N-terminal kinase (JNK), as well as increase of C/EBP homologous protein (CHOP) expression suggested that the damage caused by the treatment was too severe and cells were directed into cell death processes (FIG. 3D). Subsequently, the cytotoxicity of 3met in presence or absence of various specific UPR inhibitors was compared, which confirmed the specific role of elF2α and JNK pathways, as well as global protein synthesis in the anticancer activity of 3met (FIG. 56).

It was demonstrated that autophagic signaling played a cytoprotective role by co-incubating breast cancer cells with 3met and autophagy inhibitor, 3-methyladenine (3-MA), which resulted in the significant potentiation of the effects of 3met. Additionally, Au^{III}-metformin complexes caused dose-dependent increase of BiP expression, indicating pro-survival UPR activation in the attempts of cancer cells to counteract cellular damage. However, decreased expression of p-elF2α, as well as increase of CHOP expression suggested that the damage caused by the treatment was too severe and cells were directed into apoptosis, reflected by the significant increase in apoptotic cell population, as well as cleaved caspase 3 and cleaved PARP expression (FIGS. 49 and 50). It should be noted that metformin also induced UPR and autophagy cancer signaling at but at clinically-irrelevant concentrations.

### Inhibition of Aggressive Breast Tumor Growth In Vivo

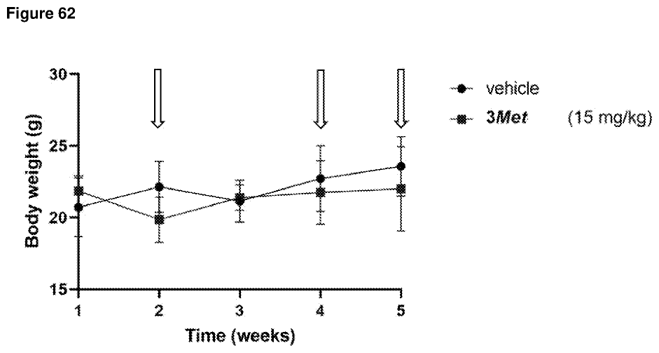

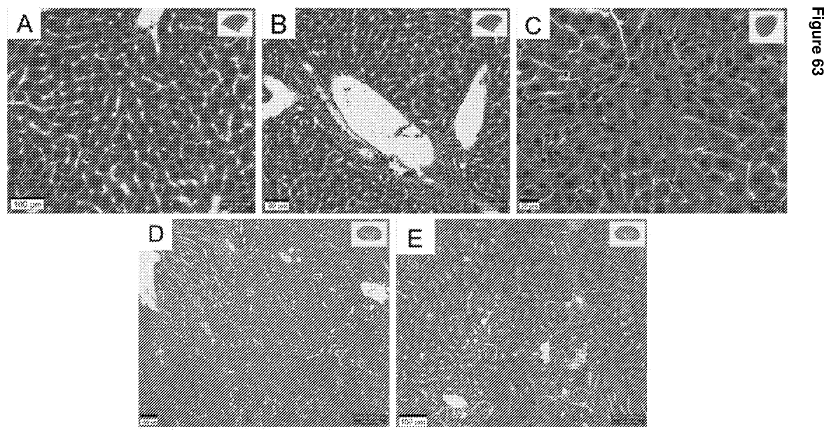

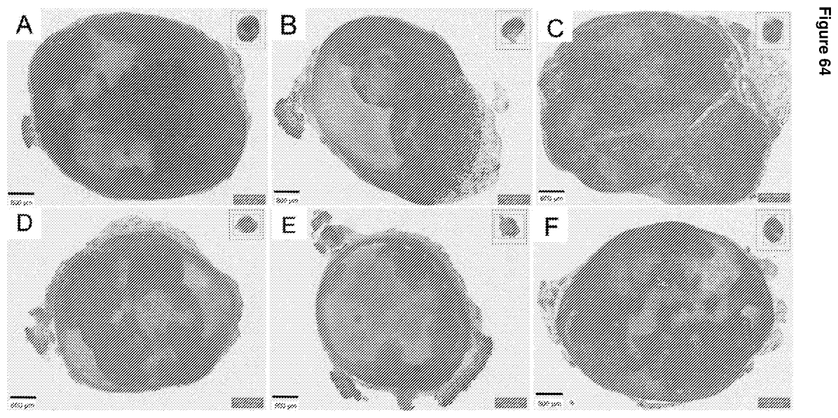

3met, which demonstrated the highest activity in vitro, was selected as a lead compound for in vivo studies. To determine the maximum tolerated dose (MTD), mice were given daily intraperitoneal injections of 3met at 5, 10, 15 and 20 mg/kg for 4 days and their body weights were monitored (FIG. 59). The tolerability of the chosen dose level was judged based on the weight loss and clinical score. All groups of mice were bright, alert and responsive; however, transient weight loss was observed at 20 mg/kg. FIG. 62 also shows that the body weights of the drug treated mice did not change in comparison with the untreated group, indicating that 3met at the chosen dose did not cause the loss of weight in treated mice. The dose-limiting toxicity included kidney and liver toxicity reflected by histopathological changes (FIG. 63). Therefore, the MTD of 3met for intraperitoneal route was determined as 15 mg/kg.

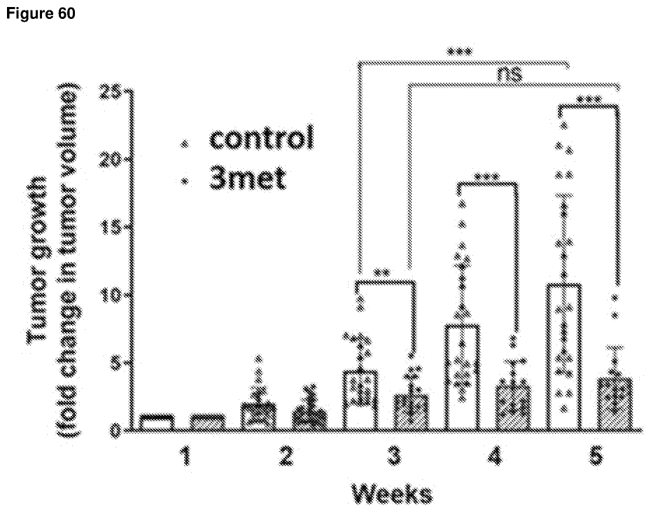

The in vivo activity of 3met was subsequently tested in female 6-week old athymic nude mice using orthotopic mammary fat pad model. Luciferase-transfected MDA-MB-231 cells were injected into 2 fat pads near pectoral nipples and 2 fat pads near inguinal nipples and tumor growth was controlled by bioluminescent imaging. Mice (7 per group) were injected with 15 mg/kg of 3met or respective vehicle (DMSO in physiological saline) intraperitoneally 3 times a week on weeks 3, 4 and 5 and sacrificed on week 6. Body weight changes reflecting upon drug toxicity are shown in FIG. 59. Importantly, 3met demonstrated marked decrease of tumor burden in comparison with a vehicle-treated group and significantly slowed down the growth of quickly growing breast tumors, with no growth observed after week 3 (FIG. 60). On the contrary, the anticancer effects of uncoordinated metformin in an MDA-MB-231 mammary fat model were negligible even at a very high dose (250 mg/kg).

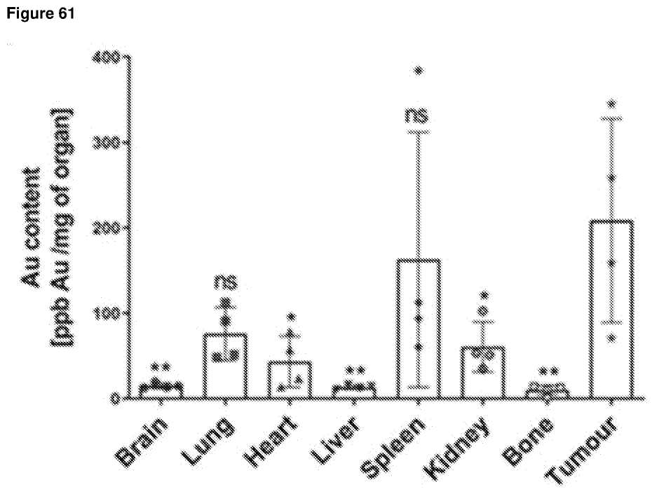

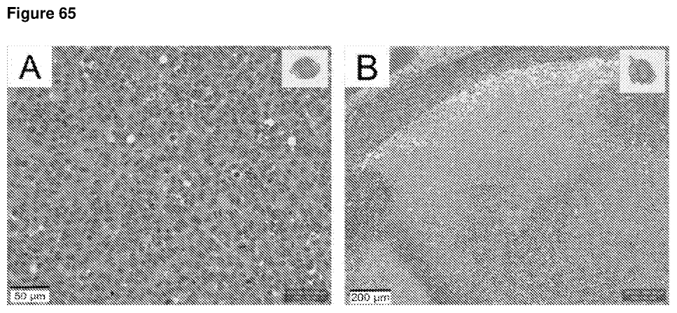

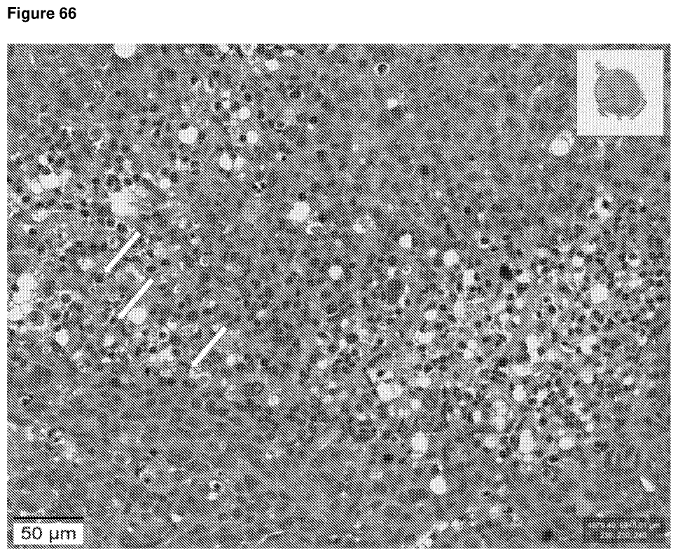

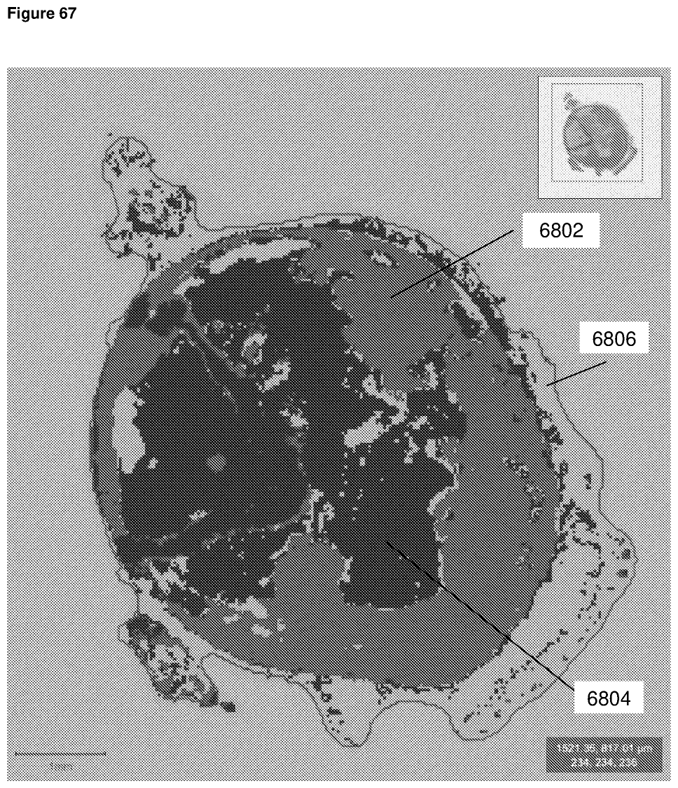

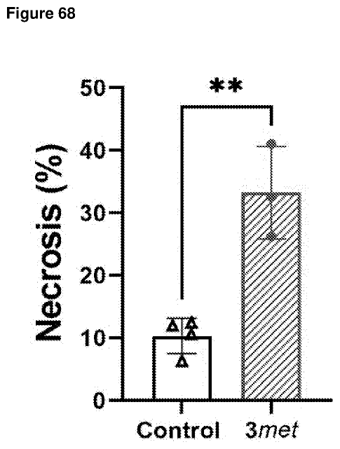

Additionally, the Au biodistribution across various organs in tumor-bearing mice was assessed. FIG. 61 demonstrates that 3met selectively accumulated in tumors. The Au content in tumors was more than 3-5 times higher than in the heart, lung, spleen and kidneys and more than 3-20 times higher than in the brain, liver and bone. This biodistribution pattern is very uncommon for small molecules and is a desirable property for novel anticancer drug candidates. Subsequently, histological changes in tumor tissues were assessed by hematoxylin & eosin (H&E) staining and the effects of 3met on tumor area and necrosis were quantified using an automated QuPath algorithm (Table 6, FIGS. 64 to 68). Tumors were represented by invasive non-specified breast carcinoma with high-grade histology G3 (3+3+3, according to the modified Bloom Richardson Grading scores), characteristic of the basal or TNBC molecular subtypes (FIG. 65). Tumors in vehicle-treated group demonstrated some areas of necrosis (10.33±1.4%) caused by high proliferative activity of aggressive breast cancer cells, while drug-treated group was characterized by significant areas of necrosis (32.25±4.27%), indicating anticancer effects of 3met. 3met-treated tumors demonstrated marked inflammatory cells infiltration, indicating enhanced immune response to the primary tumor (FIG. 66). This is an important finding since basal subtypes of breast cancers that are regulated by tumor-infiltrating immune cells were linked with improved prognosis and drug sensitivity.

TABLE 6
Analysis of H&E-stained tumor tissues of vehicle- and 3met-treated mice.

| | | | | | | | Stroma | | Tumor | |
|---|---|---|---|---|---|---|---|---|---|---|
| | Image | | Perimeter | Necrosis | Necrosis | Stroma | area | | area | Histological |
| Group | identifier | Area μm² | μm | % | area mm² | % | mm\hat{ }2 | Tumor % | mm\hat{ }2 | grade |
| Vehicle | Figure | 50129215.2 | 27380.2 | 11.98 | 6.007 | 11.27 | 5.651 | 76.7 | 38.45 | G3 (3 + 3 + 3) |
| | 64A | | | | | | | | | |
| Vehicle | Figure | 35449968.4 | 31071.1 | 10.61 | 3.764 | 19.02 | 6.746 | 69.8 | 24.76 | G3 (3 + 3 + 3) |
| | 64B | | | | | | | | | |
| Vehicle | Figure | 35621368 | 26839.7 | 6.293 | 2.241 | 12.78 | 4.552 | 80.91 | 28.82 | G3 (3 + 3 + 3) |
| | 64C | | | | | | | | | |
| 3met | Figure | 23769502 | 20891.3 | 32.52 | 7.73 | 7.363 | 1.75 | 60.11 | 14.29 | G3 (3 + 3 + 3) |
| | 64D | | | | | | | | | |
| 3met | Figure | 27637537.7 | 25672.2 | 41 | 11.33 | 23.93 | 6.613 | 34.58 | 9.556 | G3 (3 + 3 + 3) |
| | 64E | | | | | | | | | |
| 3met | Figure | 28709913 | 21813.3 | 26.24 | 7.533 | 7.293 | 2.094 | 66.37 | 19.05 | G3 (3 + 3 + 3) |
| | 64F | | | | | | | | | |

### Discussion on In Vivo Activity

Despite significant advancements in the treatment of breast cancer, triple-negative breast cancers (TNBCs) represent an unmet clinical need due to their aggressive nature and propensity to metastasize. Unlike other subtypes of breast cancer, TNBCs do not express estrogen, progesterone and Herceptin 2 receptors and cannot be treated with hormone therapies or Her2-targeting drugs, such as Trastuzumab. Therefore, the only systemic treatment that can be used for TNBCs is chemotherapy. It is known that TNBCs readily respond to currently used chemotherapeutic options, e.g. ACT regimen (anthracycline, cyclophosphamide and taxane). However, despite initial response, they quickly relapse and metastasize, which poses a serious challenge for the selection of second-line treatment options. In recent years, several classes of metal-based compounds have been developed as anticancer therapeutic agents endowed with multimodal activity against TNBCs in vitro and in vivo.

The phenotypic aggressiveness of TNBCs is related to their dependency on glucose and lipids, which cancer cells use for production of energy. It was previously shown that antidiabetic drug metformin targeted glucose metabolism in TNBCs, which made this drug particularly toxic to this group of breast cancers. However, the use of metformin for the treatment of TNBCs is hindered by its inability to effectively penetrate through cellular membranes. The approach detailed in this study was based on the conjugation of metformin and phenformin with Au^{III} pharmacophores, which are known to target multiple biomolecules, including zinc-finger domains, TrxR enzyme and various mitochondrial proteins, resulting in synergistic mitochondrial damage. It was hypothesized that cyclometalated Au^{III} scaffolds can act as multimodal prodrugs achieving targeted release of metformin and phenformin. To test this intriguing approach, a series of cyclometalated Au^{III} complexes of metformin and phenformin were investigated for their potential for treatment of TNBCs. The release of metformin was dictated by the cyclometalated fragment with the most cytotoxic complex 3met being the most efficient amongst the panel of compounds tested. 3met also displayed nanomolar cytotoxic activities and was approximately 28-fold more active than cisplatin in MDA-MB-231 cells (TNBC/basal breast cancer cell line) and more than 6000-fold cytotoxic than free metformin. 3met was also markedly more active than its cyclometalated precursor 3.

It was demonstrated that Au^{III} pharmacophores and metformin displayed synergistic action and completely shut down energy production in TNBC cells. A number of prodrugs utilize the concept of “Warburg effect” and aim to switch cancer cell metabolism from glycolysis to oxidative phosphorylation. In contrast, 3met fully inhibited mitochondrial respiration, thereby forcing cancer cells to increase glucose production via glycolysis (FIGS. 36 and 37). However, as discussed above, prolonged exposure to high concentrations of 3met resulted in a severe energetic crisis leading to the failure of breast cancer cells to protect themselves by metabolic reprogramming. Furthermore, it was demonstrated that prolonged exposure to high concentrations of 3met resulted in the inhibition of UPR and autophagy. Specifically, 3met interfered with the process of mitophagy, aimed to clear the defective mitochondria following drug-induced mitochondrial damage (FIG. 53).

Encouraged by the anticancer potential of 3met, the efficacy of this drug candidate in an orthotopic mammary fat pad model in athymic nude mice was tested, where MDA-MB-231 cells were implanted into 4 nipples, simultaneously forming 4 aggressive breast tumors (FIGS. 60, 61 and 65 to 68). In this model, implanted cancer cells match the tumor histotype of the organ, thereby providing a more realistic disease-relevant environment in contrast to commonly used xenograft models. 3met significantly reduced tumor burden in comparison to vehicle-treated mice and no tumor growth was observed after week 3. Based on these findings, 3met appears to be an effective metformin prodrug, which was able to slow the growth of invasive TNBC with subsequent activation of immune system by targeting the dependency of this cancer subtype on energy production.

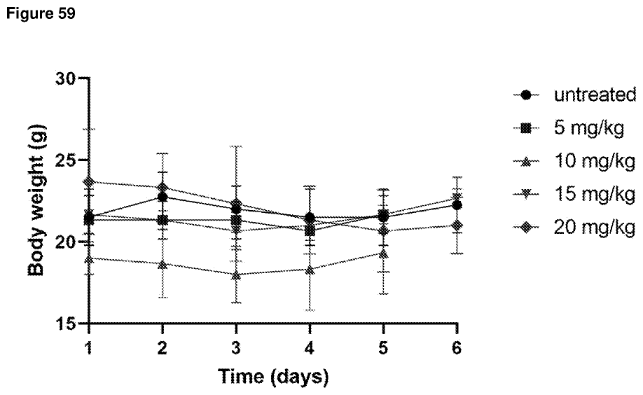

In conclusion, a new series of Au^{III} complexes was designed, featuring both energy-disrupting metformin or phenformin and TrxR-inhibiting Au^{III} species. The proposed mechanism of action of 3met is illustrated in FIG. 58. In vitro evidence demonstrated that metabolic changes caused by 3met initiated the attempts of cancer cells to protect themselves by metabolic reprogramming, UPR and mitophagy, which were successfully prevented by shutdown of mitochondrial respiration and impairment of autophagic flux, leading to the inhibition of protein degradation and apoptotic cell death. Furthermore, high degree of selectivity of novel complexes to cancer cells over healthy heart and liver cells has been observed, which is beneficial for their further preclinical development. In addition, lead drug candidate 3met halted the growth of aggressive breast tumors in a mammary fat pad breast cancer model and activated the immune response, indicating the potential benefits of this drug candidate for TNBC patients with high risk of metastasis and relapse.

### INDUSTRIAL APPLICABILITY

The compounds as defined above may be useful in as a medicament, specifically in the treatment of cancer or in the manufacture of a medicament for the treatment of cancer. The compound as defined above may be effective in treating various cancers including medulloblastoma, ewing sarcoma, osteosarcoma, rhabdomyosarcoma, fibrosarcoma connective, chondrosarcoma, neuroblastoma, esophageal cancer, head and neck cancer, gastric cancer, pancreatic cancer, glioma, bladder cancer, kidney cancer, prostate cancer, melanoma, mesothelioma, hepatocellular carcinoma, triple-negative breast cancer, colon cancer and metastatic cancer.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A compound of formula (I): 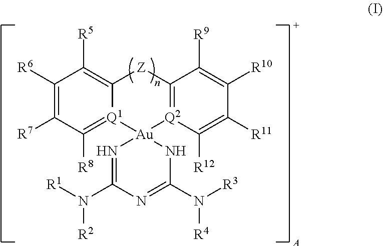
wherein,
n is 0 or 1;
Z is C, N, O, S, Si, C═O or C═S,
Q¹ and Q² for each occurrence is independently selected from C, P or N, wherein at least one of Q¹ or Q² is N;
R¹, R², R³ and R⁴ for each occurrence is independently selected from the group consisting of hydrogen, optionally substituted alkyl, optionally substituted alkenyl, and optionally substituted alkynyl, provided that at least one of R¹, R², R³ or R⁴ is an optionally substituted alkyl;
R⁵, R⁶, R⁷, R⁸, R⁹, R¹⁰, R¹¹ and R¹² for each occurrence is independently selected from the group consisting of hydrogen, —X, —OR′, —SR′, —P(R′)₂, —C(═O)OR′, C(═O)R′, —COX, —CX₃, —NO₂, —SO₃H, —SO₂R′, —N═O, optionally substituted alkyl, optionally substituted amino, optionally substituted alkyloxy, optionally substituted cycloalkyl, optionally substituted heterocycloalkyl, optionally substituted aryl, and optionally substituted heteroaryl, wherein R′ is independently hydrogen or an optionally substituted alkyl and X is halogen; and
A is tosylate (OTs⁻), mesylate (OMs⁻), BF₄⁻, NO₃⁻, Cl⁻ Br⁻, triflate (OTf⁻), ClO₄⁻, bistriflimide (TFSI⁻), BPH₄⁻, BPh₄^{F-}, tetrakis(1-imidazolyl)borate (BIm₄⁻), tetrakis(2-thienyl)borate (BTh₄⁻), HPO₄²⁻ or PF₆.

2. The compound according to claim 1, wherein Q¹ is N and Q² is C or Q¹ is C and Q² is N.

3. The compound according to claim 1, wherein R⁵, R⁶, R⁷, R⁸, R⁹, R¹⁰, R¹¹ and R¹² are hydrogen.

4. The compound according to claim 1, wherein R³ and R⁴ are hydrogen.

5. The compound according to claim 1, wherein R¹ and R² are independently optionally substituted alkyl.

6. The compound according to claim 5, wherein R¹ and R² are methyl.

7. The compound according to claim 1, wherein R¹ is hydrogen and R² is a substituted alkyl.

8. The compound according to claim 7, wherein R² is an arylalkyl.

9. The compound according to claim 8, wherein R² has the following formula (II): 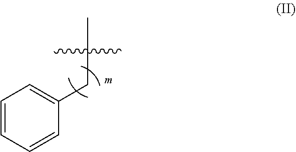
wherein m is an integer from 1 to 10.

10. The compound according to claim 9, wherein m is 2.

11. The compound according to claim 1, wherein n is 0.

12. The compound according to claim 1, wherein n is 1 and Z is C or C═O.

13. The compound according to claim 1, having the following formula (IIIa), (IIIb), (IIIc) or (IIId):

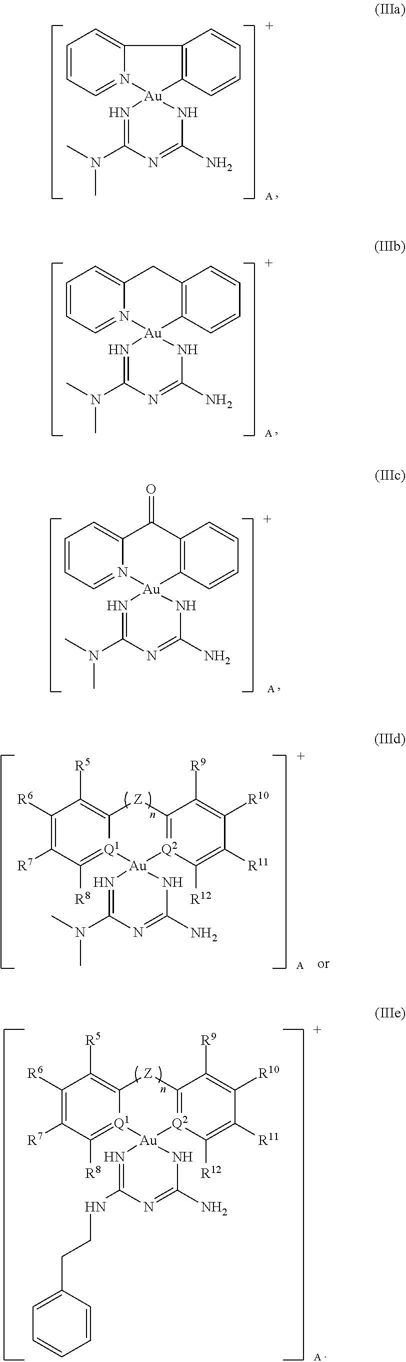

14. The compound according to claim 1, having the following structure:

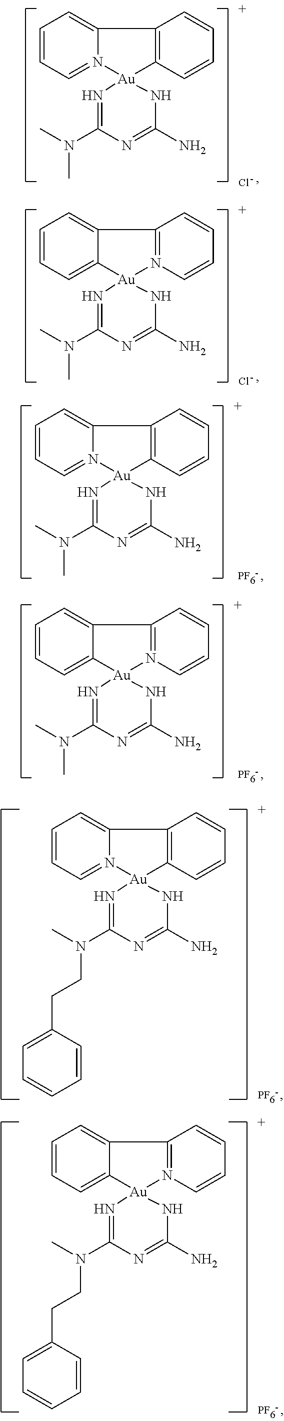

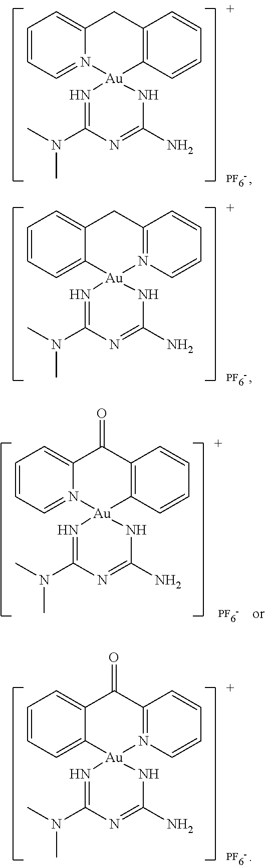

15. A method for preparing a compound according to claim 1, the method comprising the step of contacting:
a compound having the following formula (IV): 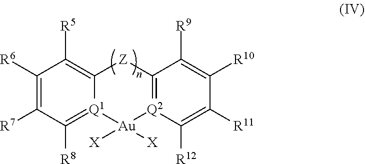
with a compound having the following formula (V): 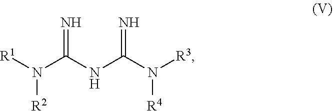
wherein
n is 0 or 1;
Z is C, N, O, S, Si, C═O or C═S,
Q¹ and Q² for each occurrence is independently selected from C, P or N, wherein at least one of Q¹ or Q² is N;
R¹, R², R³ and R⁴ for each occurrence is independently selected from the group consisting of hydrogen, optionally substituted alkyl, optionally substituted alkenyl, and optionally substituted alkynyl, provided that at least one of R¹, R², R³ or R⁴ is an optionally substituted alkyl;
R⁵, R⁶, R⁷, R⁸, R⁹, R¹⁰, R¹¹ and R¹² for each occurrence is independently selected from the group consisting of hydrogen, —X, —OR′, —SR′, —P(R′)₂, —C(═O)OR′, C(═O)R′, —COX, —CX₃, —NO₂, —SO₃H, —SO₂R′, —N═O, optionally substituted alkyl, optionally substituted amino, optionally substituted alkyloxy, optionally substituted cycloalkyl, optionally substituted heterocycloalkyl, optionally substituted aryl, and optionally substituted heteroaryl, wherein R′ is independently hydrogen or an optionally substituted alkyl and X is halogen.

16. A pharmaceutical composition comprising a compound according to claim 1 and a pharmaceutically acceptable excipient.

17. (canceled)

18. (canceled)

19. (canceled)
